SEQUENCE LISTING (153 sequences)

SEQ ID NO: 1 - protein, 483 aa, Homo sapiens
MAQADIALIGLAVMGQNLILNMNDHGFVVCAFNRTVSKVDDFLANEAKGTKVVGAQSLKEMVSKLKKPRRIILLVKAGQAVDDFIEKLVPLLDTGDIIIDGGNSEYRDTTRRCRDLKAKGILFVGSGVSGGEEGARYGPSLMPGGNKEAWPHIKTIFQGIAAKVGTGEPCCDWVGDEGAGHFVKMVHNGIEYGDMQLICEAYHLMKDVLGMAQDEMAQAFEDWNKTELDSFLIEITANILKFQDTDGKHLLPKIRDSAGQKGTGKWTAISALEYGVPVTLIGEAVFARCLSSLKDERIQASKKLKGPQKFQFDGDKKSFLEDIRKALYASKIISYAQGFMLLRQAATEFGWTLNYGGIALMWRGGCIIRSVFLGKIKDAFDRNPELQNLLLDDFFKSAVENCQDSWRRAVSTGVQAGIPMPCFTTALSFYDGYRHEMLPASLIQAQRDYFGAHTYELLAKPGQFIHTNWTGHGGTVSSSSYNA

SEQ ID NO: 2 - protein, 745 aa, Homo sapiens
MGVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 3 - protein, 646 aa, Homo sapiens
MSKGPAVGIDLGTTYSCVGVFQHGKVEIIANDQGNRTTPSYVAFTDTERLIGDAAKNQVAMNPTNTVFDAKRLIGRRFDDAVVQSDMKHWPFMVVNDAGRPKVQVEYKGETKSFYPEEVSSMVLTKMKEIAEAYLGKTVTNAVVTVPAYFNDSQRQATKDAGTIAGLNVLRIINEPTAAAIAYGLDKKVGAERNVLIFDLGGGTFDVSILTIEDGIFEVKSTAGDTHLGGEDFDNRMVNHFIAEFKRKHKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELTGIPPAPRGVPQIEVTFDIDANGILNVSAVDKSTGKENKITITNDKGRLSKEDIERMVQEAEKYKAEDEKQRDKVSSKNSLESYAFNMKATVEDEKLQGKINDEDKQKILDKCNEIINWLDKNQTAEKEEFEHQQKELEKVCNPIITKLYQSAGGMPGGMPGGFPGGGAPPSGGASSGPTIEEVD

SEQ ID NO: 4 - protein, 44 aa, Homo sapiens [sequence omitted]

SEQ ID NO: 5 - protein, 283 aa, Homo sapiens
MCIPPSYADLGKAARDIFNKGFGFGLVKLDVKTKSCSGVEFSTSGSSNTDTGKVTGTLETKYKWCEYGLTFTEKWNTDNTLGTEIAIEDQICQGLKLTFDTTFSPNTGKKSGKIKSSYKRECINLGCDVDFDFAGPAIHGSAVFGYEGWLAGYQMTFDSAKSKLTRNNFAVGYRTGDFQLHTNVNDGTEFGGSIYQKVCEDLDTSVNLAWTSGTNCTRFGIAAKYQLDPTASISAKVNNSSLIGVGYTQTLRPGVKLTLSALVDGKSINAGGHKVGLALELEA

SEQ ID NO: 6 - protein, 93 aa, Homo sapiens
MLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 7 - protein, 114 aa, Homo sapiens
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 8 - protein, 298 aa, Homo sapiens
MTDAAVSFAKDFLAGGVAAAISKTAVAPIERVKLLLQVQHASKQITADKQYKGIIDCVVRIPKEQGVLSFWRGNLANVIRYFPTQALNFAFKDKYKQIFLGGVDKRTQFWLYFAGNLASGGAAGATSLCFVYPLDFARTRLAADVGKAGAEREFRGLGDCLVKIYKSDGIKGLYQGFNVSVQGIIIYRAAYFGIYDTAKGMLPDPKNTHIVISWMIAQTVTAVAGLTSYPFDTVRRRMMMQSGRKGTDIMYTGTLDCWRKIARDEGGKAFFKGAWSNVLRGMGGAFVLVLYDEIKKYT

SEQ ID NO: 9 - protein, 213 aa, Homo sapiens [sequence omitted]

SEQ ID NO: 10 - protein, 165 aa, Homo sapiens
MVNPTVFFDIAVDGEPLGRVSFELFADKVPKTAENFRALSTGEKGFGYKGSCFHRIIPGFMCQGGDFTRHNGTGGKSIYGEKFEDENFILKHTGPGILSMANAGPNTNGSQFFICTAKTEWLDGKHVVFGKVKEGMNIVEAMERFGSRNGKTSKKITIADCGQLE

SEQ ID NO: 11 - protein, 142 aa, Homo sapiens
MSLLPVPYTEAASLSTGSTVTIKGRPLACFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDISLTKFNVSYLKR

SEQ ID NO: 12 - protein, 337 aa, Homo sapiens
MSSSPVKRQRMESALDQLKQFTTVVADTGDFHAIDEYKPQDATTNPSLILAAAQMPAYQELVEEAIAYGRKLGGSQEDQIKNAIDKLFVLFGAEILKKIPGRVSTEVDARLSFDKDAMVARARRLIELYKEAGISKDRILIKLSSTWEGIQAGKELEEQHGIHCNMTLLFSFAQAVACAEAGVTLISPFVGRILDWHVANTDKKSYEPLEDPGVKSVTKIYNYYKKFSYKTIVMGASFRNTGEIKALAGCDFLTISPKLLGELLQDNAKLVPVLSAKAAQASDLEKIHLDEKSFRWLHNEDQMAVEKLSDGIRKFAADAVKLERMLTERMFNAENGK

SEQ ID NO: 13 - protein, 126 aa, Homo sapiens
MPELAKSAPAPKKGSKKAVTKAQKKDGKKRKRSRKESYSVYVYKVLKQVHPDTGISSKAMGIMNSFVNDIFERIASEASRLAHYNKRSTITSREIQTAVRLLLPGELAKHAVSEGTKAVTKYTSSK

SEQ ID NO: 14 - protein, 574 aa, Homo sapiens
MSIQENISSLQLRSWVSKSQRDLAKSILIGAPGGPAGYLRRASVAQLTQELGTAFFQQQQLPAAMADTFLEHLCLLDIDSEPVAARSTSIIATIGPASRSVERLKEMIKAGMNIARLNFSHGSHEYHAESIANVREAVESFAGSPLSYRPVAIALDTKGPEIRTGILQGGPESEVELVKGSQVLVTVDPAFRTRGNANTVWVDYPNIVRVVPVGGRIYIDDGLISLVVQKIGPEGLVTQVENGGVLGSRKGVNLPGAQVDLPGLSEQDVRDLRFGVEHGVDIVFASFVRKASDVAAVRAALGPEGHGIKIISKIENHEGVKRFDEILEVSDGIMVARGDLGIEIPAEKVFLAQKMMIGRCNLAGKPVVCATQMLESMITKPRPTRAETSDVANAVLDGADCIMLSGETAKGNFPVEAVKMQHAIAREAEAAVYHRQLFEELRRAAPLSRDPTEVTAIGAVEAAFKCCAAAIIVLTTTGRSAQLLSRYRPRAAVIAVTRSAQAARQVHLCRGVFPLLYREPPEAIWADDVDRRVQFGIESGKLRGFLRVGDLVIVVTGWRPGSGYTNIMRVLSIS

SEQ ID NO: 15 - protein, 448 aa, Homo sapiens
MRECISVHVGQAGVQMGNACWELYCLEHGIQPDGQMPSDKTIGGGDDSFTTFFCETGAGKHVPRAVFVDLEPTVIDEIRNGPYRQLFHPEQLITGKEDAANNYARGHYTIGKEIIDPVLDRIRKLSDQCTGLQGFLVFHSFGGGTGSGFTSLLMERLSVDYGKKSKLEFSIYPAPQVSTAVVEPYNSILTTHTTLEHSDCAFMVDNEAIYDICRRNLDIERPTYTNLNRLISQIVSSITASLRFDGALNVDLTEFQTNLVPYPRIHFPLATYAPVISAEKAYHEQLSVAEITNACFEPANQMVKCDPRHGKYMACCLLYRGDVVPKDVNAAIAAIKTKRSIQFVDWCPTGFKVGINYQPPTVVPGGDLAKVQRAVCMLSNTTAIAEAWARLDHKFDLMYAKRAFVHWYVGEGMEEGEFSEAREDMAALEKDYEEVGIDSYEDEDEGEE

SEQ ID NO: 16 - protein, 561 aa, Homo sapiens
MAEQVALSRTQVCGILREELFQGDAFHQSDTHIFIIMGASGDLAKKKIYPTIWWLFRDGLLPENTFIVGYARSRLTVADIRKQSEPFFKATPEEKLKLEDFFARNSYVAGQYDDAASYQRLNSHMNALHLGSQANRLFYLALPPTVYEAVTKNIHESCMSQIGWNRIIVEKPFGRDLQSSDRLSNHISSLFREDQIYRIDHYLGKEMVQNLMVLRFANRIFGPIWNRDNIACVILTFKEPFGTEGRGGYFDEFGIIRGPGRQGGSGSESCSLSLGSLVWGPHALEPGEQGGELRRALASSVPRDVMQNHLLQMLCLVAMEKPASTNSDDVRDEKVKVLKCISEVQANNVVLGQYVGNPDGEGEATKGYLDDPTVPRGSTTATFAAVVLYVENERWDGVPFILRCGKALNERKAEVRLQFHDVAGDIFHQQCKRNELVIRVQPNEAVYTKMMTKKPGMFFNPEESELDLTYGNRYKNVKLPDAYERLILDVFCGSQMHFVRSDELREAWRIFTPLLHQIELEKPKPIPYIYGSRGPTEADELMKRVGFQYEGTYKWVNPHKL

SEQ ID NO: 17 - protein, 443 aa, Homo sapiens
MDSRAQLWGLALNKRRATLPHPGGSTNLKADPEELFTKLEKIGKGSFGEVFKGIDNRTQKVVAIKIIDLEEAEDEIEDIQQEITVLSQCDSPYVTKYYGSYLKDTKLWIIMEYLGGGSALDLLEPGPLDETQIATILREILKGLDYLHSEKKIHRDIKAANVLLSEHGEVKLADFGVAGQLTDTQIKRNTFVGTPFWMAPEVIKQSAYDSKADIWSLGITAIELARGEPPHSELHPMKVLFLIPKNNPPTLEGNYSKPLKEFVEACLNKEPSFRPTAKELLKHKFILRNAKKTSYLTELIDRYKRWKAEQSHDDSSSEDSDAETDGQASGGSDSGDWIFTIREKDPKNLENGALQPSDLDRNKMKDIPKRPFSQCLSTIISPLFAELKEKSQACGGNLGSIEELRGAIYLAEEACPGISDTMVAQLVQRLQRYSLSGGGTSSH

SEQ ID NO: 18 - protein, 154 aa, Homo sapiens [sequence omitted]

SEQ ID NO: 19 - protein, 236 aa, Homo sapiens [sequence omitted]

SEQ ID NO: 20 - protein, 145 aa, Homo sapiens [sequence omitted]

SEQ ID NO: 21 - protein, 418 aa, Homo sapiens
MAGRLPACVVDCGTGYTKLGYAGNTEPQFIIPSCIAIKESAKVGDQAQRRVMKGVDDLDFFIGDEAIEKPTYATKWPIRHGIVEDWDLMERFMEQVIFKYLRAEPEDHYFLLTEPPLNTPENREYTAEIMFESFNVPGLYIAVQAVLALAASWTSRQVGERTLTGTVIDSGDGVTHVIPVAEGYVIGSCIKHIPIAGRDITYFIQQLLRDREVGIPPEQSLETAKAVKERYSYVCPDLVKEFNKYDTDGSKWIKQYTGINAISKKEFSIDVGYERFLGPEIFFHPEFANPDFTQPISEVVDEVIQNCPIDVRRPLYKNIVLSGGSTMFRDFGRRLQRDLKRTVDARLKLSEELSGGRLKPKPIDVQVITHHMQRYAVWFGGSMLASTPEFYQVCHTKKDYEEIGPSICRHNPVFGVMS

SEQ ID NO: 22 - protein, 548 aa, Homo sapiens
MVLAELYVSDREGSDATGDGTKEKPFKTGLKALMTVGKEPFPTIYVDSQKENERWNVISKSQLKNIKKMWHREQMKSESREKKEAEDSLRREKNLEEAKKITIKNDPSLPEPKCVKIGALEGYRGQRVKVFGWVHRLRRQGKNLMFLVLRDGTGYLQCVLADELCQCYNGVLLSTESSVAVYGMLNLTPKGKQAPGGHELSCDFWELIGLAPAGGADNLINEESDVDVQLNNRHMMIRGENMSKILKARSMVTRCFRDHFFDRGYYEVTPPTLVQTQVEGGATLFKLDYFGEEAFLTQSSQLYLETCLPALGDVFCIAQSYRAEQSRTRRHLAEYTHVEAECPFLTFDDLLNRLEDLVCDVVDRILKSPAGSIVHELNPNFQPPKRPFKRMNYSDAIVWLKEHDVKKEDGTFYEFGEDIPEAPERLMTDTINEPILLCRFPVEIKSFYMQRCPEDSRLTESVDVLMPNVGEIVGGSMRIFDSEEILAGYKREGIDPTPYYWYTDQRKYGTCPHGGYGLGLERFLTWILNRYHIRDVCLYPRFVQRCTP

SEQ ID NO: 23 - protein, 445 aa, Homo sapiens
MAEYDLTTRIAHFLDRHLVFPLLEFLSVKEIYNEKELLQGKLDLLSDTNMVDFAMDVYKNLYSDDIPHALREKRTTVVAQLKQLQAETEPIVKMFEDPETTRQMQSTRDGRMLFDYLADKHGFRQEYLDTLYRYAKFQYECGNYSGAAEYLYFFRVLVPATDRNALSSLWGKLASEILMQNWDAAMEDLTRLKETIDNNSVSSPLQSLQQRTWLIHWSLFVFFNHPKGRDNIIDLFLYQPQYLNAIQTMCPHILRYLTTAVITNKDVRKRRQVLKDLVKVIQQESYTYKDPITEFVECLYVNFDFDGAQKKLRECESVLVNDFFLVACLEDFIENARLFIFETFCRIHQCISINMLADKLNMTPEEAERWIVNLIRNARLDAKIDSKLGHVVMGNNAVSPYQQVIEKTKSLSFRSQMLAMNIEKKLNQNSRSEAPNWATQDSGFY

SEQ ID NO: 24 - protein, 411 aa, Homo sapiens
MATTATMATSGSARKRLLKEEDMTKVEFETSEEVDVTPTFDTMGLREDLLRGIYAYGFEKPSAIQQRAIKQIIKGRDVIAQSQSGTGKTATFSISVLQCLDIQVRETQALILAPTRELAVQIQKGLLALGDYMNVQCHACIGGTNVGEDIRKLDYGQHVVAGTPGRVFDMIRRRSLRTRAIKMLVLDEADEMLNKGFKEQIYDVYRYLPPATQVVLISATLPHEILEMTNKFMTDPIRILVKRDELTLEGIKQFFVAVEREEWKFDTLCDLYDTLTITQAVIFCNTKRKVDWLTEKMREANFTVSSMHGDMPQKERESIMKEFRSGASRVLISTDVWARGLDVPQVSLIINYDLPNNRELYIHRIGRSGRYGRKGVAINFVKNDDIRILRDIEQYYSTQIDEMPMNVADLI

SEQ ID NO: 25 - protein, 221 aa, Homo sapiens
MSYGRPPPDVEGMTSLKVDNLTYRTSPDTLRRVFEKYGRVGDVYIPRDRYTKESRGFAFVRFHDKRDAEDAMDAMDGAVLDGRELRVQMARYGRPPDSHHSRRGPPPRRYGGGGYGRRSRSPRRRRRSRSRSRSRSRSRSRSRYSRSKSRSRTRSRSRSTSKSRSARRSKSKSSSVSRSRSRSRSRSRSRSPPPVSKRESKSRSRSKSPPKSPEEEGAVSS

SEQ ID NO: 26 - protein, 238 aa, Homo sapiens
MSRYGRYGGETKVYVGNLGTGAGKGELERAFSYYGPLRTVWIARNPPGFAFVEFEDPRDAEDAVRGLDGKVICGSRVRVELSTGMPRRSRFDRPPARRPFDPNDRCYECGEKGHYAYDCHRYSRRRRSRSRSRSHSRSRGRRYSRSRSRSRGRRSRSASPRRSRSISLRRSRSASLRRSRSGSIKGSRYFQSPSRSRSRSRSISRPRSSRSKSRSPSPKRSRSPSGSPRRSASPERMD

SEQ ID NO: 27 - protein, 529 aa, Homo sapiens
MSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 28 - protein, 911 aa, Homo sapiens
MVDYHAANQSYQYGPSSAGNGAGGGGSMGDYMAQEDDWDRDLLLDPAWEKQQRKTFTAWCNSHLRKAGTQIENIDEDFRDGLKLMLLLEVISGERLPKPERGKMRVHKINNVNKALDFIASKGVKLVSIGAEEIVDGNAKMTLGMIWTIILRFAIQDISVEETSAKEGLLLWCQRKTAPYKNVNVQNFHISWKDGLAFNALIHRHRPELIEYDKLRKDDPVTNLNNAFEVAEKYLDIPKMLDAEDIVNTARPDEKAIMTYVSSFYHAFSGAQKAETAANRICKVLAVNQENEHLMEDYEKLASDLLEWIRRTIPWLEDRVPQKTIQEMQQKLEDFRDYRRVHKPPKVQEKCQLEINFNTLQTKLRLSNRPAFMPSEGKMVSDINNGWQHLEQAEKGYEEWLLNEIRRLERLDHLAEKFRQKASIHEAWTDGKEAMLKHRDYETATLSDIKALIRKHEAFESDLAAHQDRVEQIAAIAQELNELDYYDSHNVNTRCQKICDQWDALGSLTHSRREALEKTEKQLEAIDQLHLEYAKRAAPFNNWMESAMEDLQDMFIVHTIEEIEGLISAHDQFKSTLPDADREREAILAIHKEAQRIAESNHIKLSGSNPYTTVTPQIINSKWEKVQQLVPKRDHALLEEQSKQQSNEHLRRQFASQANVVGPWIQTKMEEIGRISIEMNGTLEDQLSHLKQYERSIVDYKPNLDLLEQQHQLIQEALIFDNKHTNYTMEHIRVGWEQLLTTIARTINEVENQILTRDAKGISQEQMQEFRASFNHFDKDHGGALGPEEFKACLISLGYDVENDRQGEAEFNRIMSLVDPNHSGLVTFQAFIDFMSRETTDTDTADQVIASFKVLAGDKNFITAEELRRELPPDQAEYCIARMAPYQGPDAVPGALDYKSFSTALYGESDL

SEQ ID NO: 29 - protein, 312 aa, Homo sapiens
MANNDAVLKRLEQKGAEADQIIEYLKQQVSLLKEKAILQATLREEKKLRVENAKLKKEIEELKQELIQAEIQNGVKQIPFPSGTPLHANSMVSENVIQSTAVTTVSSGTKEQIKGGTGDEKKAKEKIEKKGEKKEKKQQSIAGSADSKPIDVSRLDLRIGCIITARKHPDADSLYVEEVDVGEIAPRTVVSGLVNHVPLEQMQNRMVILLCNLKPAKMRGVLSQAMVMCASSPEKIEILAPPNGSVPGDRITFDAFPGEPDKELNPKKKIWEQIQPDLHTNDECVATYKGVPFEVKGKGVCRAQTMSNSGIK

SEQ ID NO: 30 - protein, 101 aa, Homo sapiens
MACPLEKALDVMVSTFHKYSGKEGDKFKLNKSELKELLTRELPSFLGKRTDEAAFQKLMSNLDSNRDNEVDFQEYCVFLSCIAMMCNEFFEGFPDKQPRKK

SEQ ID NO: 31 - protein, 379 aa, Homo sapiens
MEQLSSANTRFALDLFLALSENNPAGNIFISPFSISSAMAMVFLGTRGNTAAQLSKTFHFNTVEEVHSRFQSLNADINKRGASYILKLANRLYGEKTYNFLPEFLVSTQKTYGADLASVDFQHASEDARKTINQWVKGQTEGKIPELLASGMVDNMTKLVLVNAIYFKGNWKDKFMKEATTNAPFRLNKKDRKTVKMMYQKKKFAYGYIEDLKCRVLELPYQGEELSMVILLPDDIEDESTGLKKIEEQLTLEKLHEWTKPENLDFIEVNVSLPRFKLEESYTLNSDLARLGVQDLFNSSKADLSGMSGARDIFISKIVHKSFVEVNEEGTEAAAATAGIATFCMLMPEENFTADHPFLFFIRHNSSGSILFLGRFSSP

SEQ ID NO: 32 - protein, 348 aa, Homo sapiens
MYTAIPQSGSPFPGSVQDPGLHVWRVEKLKPVPVAQENQGVFFSGDSYLVLHNGPEEVSHLHLWIGQQSSRDEQGACAVLAVHLNTLLGERPVQHREVQGNESDLFMSYFPRGLKYQEGGVESAFHKTSTGAPAAIKKLYQVKGKKNIRATERALNWDSFNTGDCFILDLGQNIFAWCGGKSNILERNKARDLALAIRDSERQGKAQVEIVTDGEEPAEMIQVLGPKPALKEGNPEEDLTADKANAQAAALYKVSDATGQMNLTKVADSSPFALELLISDDCFVLDNGLCGKIYIWKGRKANEKERQAALQVAEGFISRMQYAPNTQVEILPQGHESPIFKQFFKDWK

SEQ ID NO: 33 - protein, 663 aa, Homo sapiens
MAGMKTASGDYIDSSWELRVFVGEEDPEAESVTLRVTGESHIGGVLLKIVEQINRKQDWSDHAIWWEQKRQWLLQTHWTLDKYGILADARLFFGPQHRPVILRLPNRRALRLRASFSQPLFQAVAAICRLLSIRHPEELSLLRAPEKKEKKKKEKEPEEELYDLSKVVLAGGVAPALFRGMPAHFSDSAQTEACYHMLSRPQPPPDPLLLQRLPRPSSLSDKTQLHSRWLDSSRCLMQQGIKAGDALWLRFKYYSFFDLDPKTDPVRLTQLYEQARWDLLLEEIDCTEEEMMVFAALQYHINKLSQSGEVGEPAGTDPGLDDLDVALSNLEVKLEGSAPTDVLDSLTTIPELKDHLRIFRPRKLTLKGYRQHWVVFKETTLSYYKSQDEAPGDPIQQLNLKGCEVVPDVNVSGQKFCIKLLVPSPEGMSEIYLRCQDEQQYARWMAGCRLASKGRTMADSSYTSEVQAILAFLSLQRTGSGGPGNHPHGPDASAEGLNPYGLVAPRFQRKFKAKQLTPRILEAHQNVAQLSLAEAQLRFIQAWQSLPDFGISYVMVRFKGSRKDEILGIANNRLIRIDLAVGDVVKTWRFSNMRQWNVNWDIRQVAIEFDEHINVAFSCVSASCRIVHEYIGGYIFLSTRERARGEELDEDLFLQLTGGHEAF

SEQ ID NO: 34 - protein, 508 aa, Homo sapiens
MLRRALLCLAVAALVRADAPEEEDHVLVLRKSNFAEALAAHKYLLVEFYAPWCGHCKALAPEYAKAAGKLKAEGSEIRLAKVDATEESDLAQQYGVRGYPTIKFFRNGDTASPKEYTAGREADDIVNWLKKRTGPAATTLPDGAAAESLVESSEVAVIGFFKDVESDSAKQFLQAAEAIDDIPFGITSNSDVFSKYQLDKDGVVLFKKFDEGRNNFEGEVTKENLLDFIKHNQLPLVIEFTEQTAPKIFGGEIKTHILLFLPKSVSDYDGKLSNFKTAAESFKGKILFIFIDSDHTDNQRILEFFGLKKEECPAVRLITLEEEMTKYKPESEELTAERITEFCHRFLEGKIKPHLMSQELPEDWDKQPVKVLVGKNFEDVAFDEKKNVFVEFYAPWCGHCKQLAPIWDKLGETYKDHENIVIAKMDSTANEVEAVKVHSFPTLKFFPASADRTVIDYNGERTLDGFKKFLESGGQDGAGDDDDLEDLEEAEEPDMEEDDDQKAVKDEL

SEQ ID NO: 35 - protein, 1394 aa, Homo sapiens
MAALLRSARWLLRAGAAPRLPLSLRLLPGGPGRLHAASYLPAARAGPVAGGLLSPARLYAIAAKEKDIQEESTFSSRKISNQFDWALMRLDLSVRRTGRIPKKLLQKVFNDTCRSGGLGGSHALLLLRSCGSLLPELKLEERTEFAHRIWDTLQKLGAVYDVSHYNALLKVYLQNEYKFSPTDFLAKMEEANIQPNRVTYQRLIASYCNVGDIEGASKILGFMKTKDLPVTEAVFSALVTGHARAGDMENAENILTVMRDAGIEPGPDTYLALLNAYAEKGDIDHVKQTLEKVEKSELHLMDRDLLQIIFSFSKAGYPQYVSEILEKVTCERRYIPDAMNLILLLVTEKLEDVALQILLACPVSKEDGPSVFGSFFLQHCVTMNTPVEKLTDYCKKLKEVQMHSFPLQFTLHCALLANKTDLAKALMKAVKEEGFPIRPHYFWPLLVGRRKEKNVQGIIEILKGMQELGVHPDQETYTDYVIPCFDSVNSARAILQENGCLSDSDMFSQAGLRSEAANGNLDFVLSFLKSNTLPISLQSIRSSLLLGFRRSMNINLWSEITELLYKDGRYCQEPRGPTEAVGYFLYNLIDSMSDSEVQAKEEHLRQYFHQLEKMNVKIPENIYRGIRNLLESYHVPELIKDAHLLVESKNLDFQKTVQLTSSELESTLETLKAENQPIRDVLKQLILVLCSEENMQKALELKAKYESDMVTGGYAALINLCCRHDKVEDALNLKEEFDRLDSSAVLDTGKYVGLVRVLAKHGKLQDAINILKEMKEKDVLIKDTTALSFFHMLNGAALRGEIETVKQLHEAIVTLGLAEPSTNISFPLVTVHLEKGDLSTALEVAIDCYEKYKVLPRIHDVLCKLVEKGETDLIQKAMDFVSQEQGEMVMLYDLFFAFLQTGNYKEAKKIIETPGIRARSARLQWFCDRCVANNQVETLEKLVELTQKLFECDRDQMYYNLLKLYKINGDWQRADAVWNKIQEENVIPREKTLRLLAEILREGNQEVPFDVPELWYEDEKHSLNSSSASTTEPDFQKDILIACRLNQKKGAYDIFLNAKEQNIVFNAETYSNLIKLLMSEDYFTQAMEVKAFAETHIKGFTLNDAANSRLIITQVRRDYLKEAVTTLKTVLDQQQTPSRLAVTRVIQALAMKGDVENIEVVQKMLNGLEDSIGLSKMVFINNIALAQIKNNNIDAAIENIENMLTSENKVIEPQYFGLAYLFRKVIEEQLEPAVEKISIMAERLANQFAIYKPVTDFFLQLVDAGKVDDARALLQRCGAIAEQTPILLLFLLRNSRKQGKASTVKSVLELIPELNEKEEAYNSLMKSYVSEKDVTSAKALYEHLTAKNTKLDDLFLKRYASLLKYAGEPVPFIEPPESFEFYAQQLRKLRENSS

SEQ ID NO: 36 - protein, 788 aa, Homo sapiens
MAMESTATAAVAAELVSADKIEDVPAPSTSADKVESLDVDSEAKKLLGLGQKHLVMGDIPAAVNAFQEAASLLGKKYGETANECGEAFFFYGKSLLELARMENGVLGNALEGVHVEEEEGEKTEDESLVENNDNIDEEAREELREQVYDAMGEKEEAKKTEDKSLAKPETDKEQDSEMEKGGREDMDISKSAEEPQEKVDLTLDWLTETSEEAKGGAAPEGPNEAEVTSGKPEQEVPDAEEEKSVSGTDVQEECREKGGQEKQGEVIVSIEEKPKEVSEEQPVVTLEKQGTAVEVEAESLDPTVKPVDVGGDEPEEKVVTSENEAGKAVLEQLVGQEVPPAEESPEVTTEAAEASAVEAGSEVSEKPGQEAPVLPKDGAVNGPSVVGDQTPIEPQTSIERLTETKDGSGLEEKVRAKLVPSQEETKLSVEESEAAGDGVDTKVAQGATEKSPEDKVQIAANEETQEREEQMKEGEETEGSEEDDKENDKTEEMPNDSVLENKSLQENEEEEIGNLELAWDMLDLAKIIFKRQETKEAQLYAAQAHLKLGEVSVESENYVQAVEEFQSCLNLQEQYLEAHDRLLAETHYQLGLAYGYNSQYDEAVAQFSKSIEVIENRMAVLNEQVKEAEGSSAEYKKEIEELKELLPEIREKIEDAKESQRSGNVAELALKATLVESSTSGFTPGGGGSSVSMIASRKPTDGASSSNCVTDISHLVRKKRKPEEESPRKDDAKKAKQEPEVNGGSGDAVPSGNEVSENMEEEAENQAESRAAVEGTVEAGATVESTAC

SEQ ID NO: 37 - protein, 249 aa, Homo sapiens
MAMLRVQPEAQAKVDVFREDLCTKTENLLGSYFPKKISELDAFLKEPALNEANLSNLKAPLDIPVPDPVKEKEKEERKKQQEKEDKDEKKKGEDEDKGPPCGPVNCNEKIVVLLQRLKPEIKDVIEQLNLVTTWLQLQIPRIEDGNNFGVAVQEKVFELMTSLHTKLEGFHTQISKYFSERGDAVTKAAKQPHVGDYRQLVHELDEAEYRDIRLMVMEIRNAYAVLYDIILKNFEKLKKPRGETKGMIY

SEQ ID NO: 38 - protein, 292 aa, Homo sapiens
MVLLSTLGIVFQGEGPPISSCDTGTMANCERTFIAIKPDGVQRGLVGEIIKRFEQKGFRLVGLKFMQASEDLLKEHYVDLKDRPFFAGLVKYMHSGPVVAMVWEGLNVVKTGRVMLGETNPADSKPGTIRGDFCIQVGRTMANLERTFIAIKPDGVQRGLVGEIIKRFEQKGFRLVAMKFLRASEEHLKQHYIDLKDRPFFPGLVKYMNSGPVVAMVWEGLNVVKTGRVMLGETNPADSKPGTIRGDFCIQVGRNIIHGSDSVKSAEKEISLWFKPEELVDYKSCAHDWVYE

SEQ ID NO: 39 - protein, 188 aa, Homo sapiens
MDVNIAPLRAWDDFFPGSDRFARPDFRDISKWNNRVVSNLLYYQTNYLVVAAMMISIVGFLSPFNMILGGIVVVLVFTGFVWAAHNKDVLRRMKKRYPTTFVMVVMLASYFLISMFGGVMVFVFGITFPLLLMFIHASLRLRNLKNKLENKMEGIGLKRTPMGIVLDALEQQEEGINRLTDYISKVKE

SEQ ID NO: 40 - protein, 443 aa, Homo sapiens
MAENDVDNELLDYEDDEVETAAGGDGAEAPAKKDVKGSYVSIHSSGFRDFLLKPELLRAIVDCGFEHPSEVQHECIPQAILGMDVLCQAKSGMGKTAVFVLATLQQLEPVTGQVYLGRVLGRGFWLGLVSVLVMCHTRELAFQISKEYERFSKYMPNVKVAVFFGGLSIKKDEEVLKKNCPHIVVGTPGRILALARNKSLNLKHIKHFILDECDKMLEQLDMRRDVQEIFRMTPHEKQVMMFSATLSKEIRPVCRKFMQDPMEIFVDDETKLTLHGLQQYYVKLKDNEKNRKLFDLLDVLEFNQVVIFVKSVQRCIALAQLLVEQNFPAIAIHRGMPQEERLSRYQQFKDFQRRILVATNLFGRGMDIERVNIAFNYDMPEDSDTYLHRVARAGRFGTKGLAITFVSDENDAKILNDVQDRFEVNISELPDEIDISSYIEQTR

SEQ ID NO: 41 - protein, 491 aa, Homo sapiens
MASRCWRWWGWSAWPRTRLPPAGSTPSFCHHFSTQEKTPQICVVGSGPAGFYTAQHLLKHPQAHVDIYEKQPVPFGLVRFGVAPDHPEVKNVINTFTQTAHSGRCAFWGNVEVGRDVTVPELREAYHAVVLSYGAEDHRALEIPGEELPGVCSARAFVGWYNGLPENQELEPDLSCDTAVILGQGNVALDVARILLTPPEHLERTDITKAALGVLRQSRVKTVWLVGRRGPLQVAFTIKELREMIQLPGARPILDPVDFLGLQDKIKEVPRPRKRLTELLLRTATEKPGPAEAARQASASRAWGLRFFRSPQQVLPSPDGRRAAGVRLAVTRLEGVDEATRAVPTGDMEDLPCGLVLSSIGYKSRPVDPSVPFDSKLGVIPNVEGRVMDVPGLYCSGWVKRGPTGVIATTMTDSFLTGQMLLQDLKAGLLPSGPRPGYAAIQALLSSRGVRPVSFSDWEKLDAEEVARGQGTGKPREKLVDPQEMLRLLGH

SEQ ID NO: 42 - protein, 439 aa, Homo sapiens
MNLLPNIESPVTRQEKMATVWDEAEQDGIGEEVLKMSTEEIIQRTRLLDSEIKIMKSEVLRVTHELQAMKDKIKENSEKIKVNKTLPYLVSNVIELLDVDPNDQEEDGANIDLDSQRKGKCAVIKTSTRQTYFLPVIGLVDAEKLKPGDLVGVNKDSYLILETLPTEYDSRVKAMEVDERPTEQYSDIGGLDKQIQELVEAIVLPMNHKEKFENLGIQPPKGVLMYGPPGTGKTLLARACAAQTKATFLKLAGPQLVQMFIGDGAKLVRDAFALAKEKAPSIIFIDELDAIGTKRFDSEKAGDREVQRTMLELLNQLDGFQPNTQVKVIAATNRVDILDPALLRSGRLDRKIEFPMPNEEARARIMQIHSRKMNVSPDVNYEELARCTDDFNGAQCKAVCVEAGMIALRRGATELTHEDYMEGILEVQAKKKANLQYYA

SEQ ID NO: 43 - protein, 679 aa, Homo sapiens
MISASRAAAARLVGAAASRGPTAARHQDSWNGLSHEAFRLVSRRDYASEAIKGAVVGIDLGTTNSCVAVMEGKQAKVLENAEGARTTPSVVAFTADGERLVGMPAKRQAVTNPNNTFYATKRLIGRRYDDPEVQKDIKNVPFKIVRASNGDAWVEAHGKLYSPSQIGAFVLMKMKETAENYLGHTAKNAVITVPAYFNDSQRQATKDAGQISGLNVLRVINEPTAAALAYGLDKSEDKVIAVYDLGGGTFDISILEIQKGVFEVKSTNGDTFLGGEDFDQALLRHIVKEFKRETGVDLTKDNMALQRVREAAEKAKCELSSSVQTDINLPYLTMDSSGPKHLNMKLTRAQFEGIVTDLIRRTIAPCQKAMQDAEVSKSDIGEVILVGGMTRMPKVQQTVQDLFGRAPSKAVNPDEAVAIGAAIQGGVLAGDVTDVLLLDVTPLSLGIETLGGVFTKLINRNTTIPTKKSQVFSTAADGQTQVEIKVCQGEREMAGDNKLLGQFTLIGIPPAPRGVPQIEVTFDIDANGIVHVSAKDKGTGREQQIVIQSSGGLSKDDIENMVKNAEKYAEEDRRKKERVEAVNMAEGIIHDTETKMEEFKDQLPADECNKLKEEISKMRELLARKDSETGENIRQAASSLQQASLKLFEMAYKKMASEREGSGSSGTGEQKEDQKEEKQ

SEQ ID NO: 44 - protein, 711 aa, Homo sapiens
MSDYSTGGPPPGPPPPAGGGGGAGGAGGGPPPGPPGAGDRGGGGPGGGGPGGGSAGGPSQPPGGGGPGIRKDAFADAVQRARQIAAKIGGDAATTVNNSTPDFGFGGQKRQLEDGDQPESKKLASQGDSISSQLGPIHPPPRTSMTEEYRVPDGMVGLIIGRGGEQINKIQQDSGCKVQISPDSGGLPERSVSLTGAPESVQKAKMMLDDIVSRGRGGPPGQFHDNANGGQNGTVQEIMIPAGKAGLVIGKGGETIKQLQERAGVKMILIQDGSQNTNVDKPLRIIGDPYKVQQACEMVMDILRERDQGGFGDRNEYGSRIGGGIDVPVPRHSVGVVIGRSGEMIKKIQNDAGVRIQFKQDDGTGPEKIAHIMGPPDRCEHAARIINDLLQSLRSGPPGPPGGPGMPPGGRGRGRGQGNWGPPGGEMTFSIPTHKCGLVIGRGGENVKAINQQTGAFVEISRQLPPNGDPNFKLFIIRGSPQQIDHAKQLIEEKIEGPLCPVGPGPGGPGPAGPMGPFNPGPFNQGPPGAPPHAGGPPPHQYPPQGWGNTYPQWQPPAPHDPSKAAAAAADPNAAWAAYYSHYYQQPPGPVPGPAPAPAAPPAQGEPPQPPPTGQSDYTKAWEEYYKKIGQQPQQPGAPPQQDYTKAWEEYYKKQAQVATGGGPGAPPGSQPDYSAAWAEYYRQQAAYYGQTPGPGGPQPPPTQQGQQQAQ

SEQ ID NO: 45 - protein, 455 aa, Homo sapiens
MTTMAAATLLRATPHFSGLAAGRTFLLQGLLRLLKAPALPLLCRGLAVEAKKTYVRDKPHVNVGTIGHVDHGKTTLTAAITKILAEGGGAKFKKYEEIDNAPEERARGITINAAHVEYSTAARHYAHTDCPGHADYVKNMITGTAPLDGCILVVAANDGPMPQTREHLLLARQIGVEHVVVYVNKADAVQDSEMVELVELEIRELLTEFGYKGEETPVIVGSALCALEGRDPELGLKSVQKLLDAVDTYIPVPARDLEKPFLLPVEAVYSVPGRGTVVTGTLERGILKKGDECELLGHSKNIRTVVTGIEMFHKSLERAEAGDNLGALVRGLKREDLRRGLVMVKPGSIKPHQKVEAQVYILSKEEGGRHKPFVSHFMPVMFSLTWDMACRIILPPEKELAMPGEDLKFNLILRQPMILEKGQRFTLRDGNRTIGTGLVTNTLAMTEEEKNIKWG

SEQ ID NO: 46 - protein, 307 aa, Homo sapiens
MSLKLQASNVTNKNDPKSINSRVFIGNLNTALVKKSDVETIFSKYGRVAGCSVHKGYAFVQYSNERHARAAVLGENGRVLAGQTLDINMAGEPKPDRPKGLKRAASAIYSGYIFDYDYYRDDFYDRLFDYRGRLSPVPVPRAVPVKRPRVTVPLVRRVKTNVPVKLFARSTAVTTSSAKIKLKSSELQAIKTELTQIKSNIDALLSRLEQIAAEQKANPDGKKKGDGGGASGGGGGGGGSGGGGSGGGGGGGSSRPPAPQENTTSEAGLPQGEARTRDDGDEEGLLTHSEEELEHSQDTDADDGALQ

SEQ ID NO: 47 - protein, 293 aa, Homo sapiens [sequence omitted]

SEQ ID NO: 48 - protein, 589 aa, Homo sapiens
MSDSGSYGQSGGEQQSYSTYGNPGSQGYGQASQSYSGYGQTTDSSYGQNYSGYSSYGQSYSQSYGGYENQKQSSYSQQPYNNQGQQQNMESSGSQGGRAPSYDQPDYGQQDSYDQQSGYDQHQGSYDEQSNYDQQHDSYSQNQQSYHSQRENYSHHTQDDRRDVSRYGEDNRGYGGSQGGGRGRGGYDKDGRGPMTGSSGGDRGGFKNFGGHRDYGPRTDADSESDNSDNNTIFVQGLGEGVSTDQVGEFFKQIGIIKTNKKTGKPMINLYTDKDTGKPKGEATVSFDDPPSAKAAIDWFDGKEFHGNIIKVSFATRRPEFMRGGGSGGGRRGRGGYRGRGGFQGRGGDPKSGDWVCPNPSCGNMNFARRNSCNQCNEPRPEDSRPSGGDFRGRGYGGERGYRGRGGRGGDRGGYGGDRSGGGYGGDRSSGGGYSGDRSGGGYGGDRSGGGYGGDRGGGYGGDRGGGYGGDRGGGYGGDRGGYGGDRGGGYGGDRGGYGGDRGGYGGDRGGYGGDRGGYGGDRSRGGYGGDRGGGSGYGGDRSGGYGGDRSGGGYGGDRGGGYGGDRGGYGGKMGGRNDYRNDQRNRPY

SEQ ID NO: 49 - protein, 432 aa, Homo sapiens
MPARPGRLLPLLARPAALTALLLLLLGHGGGGRWGARAQEAAAAAADGPPAADGEDGQDPHSKHLYTADMFTHGIQSAAHFVMFFAPWCGHCQRLQPTWNDLGDKYNSMEDAKVYVAKVDCTAHSDVCSAQGVRGYPTLKLFKPGQEAVKYQGPRDFQTLENWMLQTLNEEPVTPEPEVEPPSAPELKQGLYELSASNFELHVAQGDHFIKFFAPWCGHCKALAPTWEQLALGLEHSETVKIGKVDCTQHYELCSGNQVRGYPTLLWFRDGKKVDQYKGKRDLESLREYVESQLQRTETGATETVTPSEAPVLAAEPEADKGTVLALTENNFDDTIAEGITFIKFYAPWCGHCKTLAPTWEELSKKEFPGLAGVKIAEVDCTAERNICSKYSVRGYPTLLLFRGGKKVSEHSGGRDLDSLHRFVLSQAKDEL

SEQ ID NO: 50 - protein, 401 aa, Homo sapiens
MEPGRRGAAALLALLCVACALRAGRAQYERYSFRSFPRDELMPLESAYRHALDKYSGEHWAESVGYLEISLRLHRLLRDSEAFCHRNCSAAPQPEPAAGLASYPELRLFGGLLRRAHCLKRCKQGLPAFRQSQPSREVLADFQRREPYKFLQFAYFKANNLPKAIAAAHTFLLKHPDDEMMKRNMAYYKSLPGAEDYIKDLETKSYESLFIRAVRAYNGENWRTSITDMELALPDFFKAFYECLAACEGSREIKDFKDFYLSIADHYVEVLECKIQCEENLTPVIGGYPVEKFVATMYHYLQFAYYKLNDLKNAAPCAVSYLLFDQNDKVMQQNLVYYQYHRDTWGLSDEHFQPRPEAVQFFNVTTLQKELYDFAKENIMDDDEGEVVEYVDDLLELEETS

SEQ ID NO: 51 - protein, 215 aa, Homo sapiens
MVLLTMIARVADGLPLAASMQEDEQSGRDLQQYQSQAKQLFRKLNEQSPTRCTLEAGAMTFHYIIEQGVCDLVLCEAAFPKTLAFAYLEDLHSEFDEQHGKKVPTVSRPYSFIEFDTFIQKTKKLYIDSCARRNLGSINTELQDVQRIMVANIEEVLQRGEALSALDSKANNLSSLSKKYRQDAKYLNMHSTYAKLAAVAVFFIMLIVYVRFWWL

SEQ ID NO: 52 - protein, 466 aa, Homo sapiens
MSTRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRETNLDSLPLVDTHSKRTLLIKTVETRDGQVINETSQHHDDLE

SEQ ID NO: 53 - protein, 248 aa, Homo sapiens
MSYDRAITVFSPDGHLFQVEYAQEAVKKGSTAVGVRGRDIVVLGVEKKSVAKLQDERTVRKICALDDNVCMAFAGLTADARIVINRARVECQSHRLTVEDPVTVEYITRYIASLKQRYTQSNGRRPFGISALIVGFDFDGTPRLYQTDPSGTYHAWKANAIGRGAKSVREFLEKNYTDEAIETDDLTIKLVIKALLEVVQSGGKNIELAVMRRDQSLKILNPEEIEKYVAEIEKEKEENEKKKQKKAS

SEQ ID NO: 54 - protein, 145 aa, Homo sapiens [sequence omitted]

SEQ ID NO: 55 - protein, 417 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 56 - protein, 187 aa, Homo sapiens
MPVDLSKWSGPLSLQEVDEQPQHPLHVTYAGAAVDELGKVLTPTQVKNRPTSISWDGLDSGKLYTLVLTDPDAPSRKDPKYREWHHFLVVNMKGNDISSGTVLSDYVGSGPPKGTGLHRYVWLVYEQDRPLKCDEPILSNRSGDHRGKFKVASFRKKYELRAPVAGTCYQAEWDDYVPKLYEQLSGK

SEQ ID NO: 57 - protein, 353 aa, Homo sapiens
MGSGGRSAQVSTGQRAWLLPCRFLKNTMSNGYEDHMAEDCRGDIGRTNLIVNYLPQNMTQDELRSLFSSIGEVESAKLIRDKVAGHSLGYGFVNYVTAKDAERAINTLNGLRLQSKTIKVSYARPSSEVIKDANLYISGLPRTMTQKDVEDMFSRFGRIINSRVLVDQTTGLSRGVAFIRFDKRSEAEEAITSFNGHKPPGSSEPITVKFAANPNQNKNVALLSQLYHSPARRFGGPVHHQAQRFRFSPMGVDHMSGLSGVNVPGNASSGWCIFIYNLGQDADEGILWQMFGPFGAVTNVKVIRDFNTNKCKGFGFVTMTNYEEAAMAIASLNGYRLGDKILQVSFKTNKSHK

SEQ ID NO: 58 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 59 - protein, 437 aa, Homo sapiens
MPKFKAARGVGGQEKHAPLADQILAGNAVRAGVREKRRGRGTGEAEEEYVGPRLSRRILQQARQQQEELEAEHGTGDKPAAPRERTTRLGPRMPQDGSDDEDEEWPTLEKAATMTAAGHHAEVVVDPEDERAIEMFMNKNPPARRTLADIIMEKLTEKQTEVETVMSEVSGFPMPQLDPRVLEVYRGVREVLSKYRSGKLPKAFKIIPALSNWEQILYVTEPEAWTAAAMYQATRIFASNLKERMAQRFYNLVLLPRVRDDVAEYKRLNFHLYMALKKALFKPGAWFKGILIPLCESGTCTLREAIIVGSIITKCSIPVLHSSAAMLKIAEMEYSGANSIFLRLLLDKKYALPYRVLDALVFHFLGFRTEKRELPVLWHQCLLTLVQRYKADLATDQKEALLELLRLQPHPQLSPEIRRELQSAVPRDVEDVPITVE

SEQ ID NO: 60 - protein, 338 aa, Homo sapiens
MLSALARPASAALRRSFSTSAQNNAKVAVLGASGGIGQPLSLLLKNSPLVSRLTLYDIAHTPGVAADLSHIETKAAVKGYLGPEQLPDCLKGCDVVVIPAGVPRKPGMTRDDLFNTNATIVATLTAACAQHCPEAMICVIANPVNSTIPITAEVFKKHGVYNPNKIFGVTTLDIVRANTFVAELKGLDPARVNVPVIGGHAGKTIIPLISQCTPKVDFPQDQLTALTGRIQEAGTEVVKAKAGAGSATLSMAYAGARFVFSLVDAMNGKEGVVECSFVKSQETECTYFSTPLLLGKKGIEKNLGIGKVSSFEEKMISDAIPELKASIKKGEDFVKTLK

SEQ ID NO: 61 - protein, 90 aa, Homo sapiens
MPKRKAEGDAKGDKAKVKDEPQRRSARLSAKPAPPKPEPKPKKAPAKKGEKVPKGKKGKADAGKEGNNPAENGDAKTDQAQKAEGAGDAK

SEQ ID NO: 62 - protein, 453 aa, Homo sapiens
MKLLTRAGSFSRFYSLKVAPKVKATAAPAGAPPQPQDLEFTKLPNGLVIASLENYSPVSRIGLFIKAGSRYEDFSNLGTTHLLRLTSSLTTKGASSFKITRGIEAVGGKLSVTATRENMAYTVECLRGDVDILMEFLLNVTTAPEFRRWEVADLQPQLKIDKAVAFQNPQTHVIENLHAAAYRNALANPLYCPDYRIGKVTSEELHYFVQNHFTSARMALIGLGVSHPVLKQVAEQFLNMRGGLGLSGAKANYRGGEIREQNGDSLVHAAFVAESAVAGSAEANAFSVLQHVLGAGPHVKRGSNTTSHLHQAVAKATQQPFDVSAFNASYSDSGLFGIYTISQATAAGDVIKAAYNQVKTIAQGNLSNTDVQAAKNKLKAGYLMSVESSECFLEEVGSQALVAGSYMPPSTVLQQIDSVANADIINAAKKFVSGQKSMAASGNLGHTPFVDEL

SEQ ID NO: 63 - protein, 309 aa, Homo sapiens
MDEKVFTKELDQWIEQLNECKQLSESQVKSLCEKAKEILTKESNVQEVRCPVTVCGDVHGQFHDLMELFRIGGKSPDTNYLFMGDYVDRGYYSVETVTLLVALKVRYRERITILRGNHESRQITQVYGFYDECLRKYGNANVWKYFTDLFDYLPLTALVDGQIFCLHGGLSPSIDTLDHIRALDRLQEVPHEGPMCDLLWSDPDDRGGWGISPRGAGYTFGQDISETFNHANGLTLVSRAHQLVMEGYNWCHDRNVVTIFSAPNYCYRCGNQAAIMELDDTLKYSFLQFDPAPRRGEPHVTRRTPDYFL

SEQ ID NO: 64 - protein, 226 aa, Homo sapiens [sequence omitted]

SEQ ID NO: 65 - protein, 492 aa, Homo sapiens
MRRDLREKLVWVCRPLAPVEVPANISSDFQPCSPTSPAHSLSRKSPIMYPSTTMANAPGLVSCTFFLAVNGLYSSSDDVIELTPSNFNREVIQSDSLWLVEFYAPWCGHCQRLTPEWKKAATALKDVVKVGAVDADKHHSLGGQYGVQGFPTIKIFGSNKNRPEDYQGGRTGEAIVDAALSALRQLVKDRLGGRSGGYSSGKQGRSDSSSKKDVIELTDDSFDKNVLDSEDVWMVEFYAPWCGHCKNLEPEWAAAASEVKEQTKGKVKLAAVDATVNQVLASRYGIRGFPTIKIFQKGESPVDYDGGRTRSDIVSRALDLFSDNAPPPELLEIINEDIAKRTCEEHQLCVVAVLPHILDTGAAGRNSYLEVLLKLADKYKKKMWGWLWTEAGAQSELETALGIGGFGYPAMAAINARKMKFALLKGSFSEQGINEFLRELSFGRGSTAPVGGGAFPTIVEREPWDGRDGELPVEDDIDLSDVELDDLGKDEL

SEQ ID NO: 66 - protein, 664 aa, Homo sapiens
METPSQRRATRSGAQASSTPLSPTRITRLQEKEDLQELNDRLAVYIDRVRSLETENAGLRLRITESEEVVSREVSGIKAAYEAELGDARKTLDSVAKERARLQLELSKVREEFKELKARNTKKEGDLIAAQARLKDLEALLNSKEAALSTALSEKRTLEGELHDLRGQVAKLEAALGEAKKQLQDEMLRRVDAENRLQTMKEELDFQKNIYSEELRETKRRHETRLVEIDNGKQREFESRLADALQELRAQHEDQVEQYKKELEKTYSAKLDNARQSAERNSNLVGAAHEELQQSRIRIDSLSAQLSQLQKQLAAKEAKLRDLEDSLARERDTSRRLLAEKEREMAEMRARMQQQLDEYQELLDIKLALDMEIHAYRKLLEGEEERLRLSPSPTSQRSRGRASSHSSQTQGGGSVTKKRKLESTESRSSFSQHARTSGRVAVEEVDEEGKFVRLRNKSNEDQSMGNWQIKRQNGDDPLLTYRFPPKFTLKAGQVVTIWAAGAGATHSPPTDLVWKAQNTWGCGNSLRTALINSTGEEVAMRKLVRSVTVVEDDEDEDGDDLLHHHHGSHCSSSGDPAEYNLRSRTVLCGTCGQPADKASASGSGAQVGGPISSGSSASSVTVTRSYRSVGGSGGGSFGDNLVTRSYLLGNSSPRTQSPQNCSIM

SEQ ID NO: 67 - protein, 464 aa, Homo sapiens
MLATRRLLGWSLPARVSVRFSGDTTAPKKTSFGSLKDEDRIFTNLYGRHDWRLKGSLSRGDWYKTKEILLKGPDWILGEIKTSGLRGRGGAGFPTGLKWSFMNKPSDGRPKYLVVNADEGEPGTCKDREILRHDPHKLLEGCLVGGRAMGARAAYIYIRGEFYNEASNLQVAIREAYEAGLIGKNACGSGYDFDVFVVRGAGAYICGEETALIESIEGKQGKPRLKPPFPADVGVFGCPTTVANVETVAVSPTICRRGGTWFAGFGRERNSGTKLFNISGHVNHPCTVEEEMSVPLKELIEKHAGGVTGGWDNLLAVIPGGSSTPLIPKSVCETVLMDFDALVQAQTGLGTAAVIVMDRSTDIVKAIARLIEFYKHESCGQCTPCREGVDWMNKVMARFVRGDARPAEIDSLWEISKQIEGHTICALGDGAAWPVQGLIRHFRPELEERMQRFAQQHQARQAAS

SEQ ID NO: 68 - protein, 123 aa, Homo sapiens
SDVLELTDDNFESRISDTGSAGLMLVEFFAPWCGHCKRLAPEYEAAATRLKGIVPLAKVDCTANTNTCNKYGVSGYPTLKIFRDGEEAGAYDGPRTAGVSSYFVLHISLTSLRTRLWHIQSKK

SEQ ID NO: 69 - protein, 247 aa, Homo sapiens
MADTTPNGPQGAGAVQFMMTNKLDTAMWLSRLFTVYCSALFVLPLLGLHEAASFYQRALLANALTSALRLHQRLPHFQLSRAFLAQALLEDSCHYLLYSLIFVNSYPVTMSIFPVLLFSLLHAATYTKKVLDARGSNSLPLLRSVLDKLSANQQNILKFIACNEIFLMPATVFMLFSGQGSLLQPFIYYRFLTLRYSSRRNPYCRTLFNELRIVVEHIIMKPACPLFVRRLCLQSIAFISRLAPTVP

SEQ ID NO: 70 - protein, 339 aa, Homo sapiens
MAVAQQLRAESDFEQLPDDVAISANIADIEEKRGFTSHFVFVIEVKTKGGSKYLIYRRYRQFHALQSKLEERFGPDSKSSALACTLPTLPAKVYVGVKQEIAEMRIPALNAYMKSLLSLPVWVLMDEDVRIFFYQSPYDSEQVPQALRRLRPRTRKVKSVSPQGNSVDRMAAPRAEALFDFTGNSKLELNFKAGDVIFLLSRINKDWLEGTVRGATGIFPLSFVKILKDFPEEDDPTNWLRCYYYEDTISTIKDIAVEEDLSSTPLLKDLLELTRREFQREDIALNYRDAEGDLVRLLSDEDVALMVRQARGLPSQKRLFPWKLHITQKDNYRVYNTMP

SEQ ID NO: 71 - protein, 256 aa, Homo sapiens
MRPAALRGALLGCLCLALLCLGGADKRLRDNHEWKKLIMVQHWPETVCEKIQNDCRDPPDYWTIHGLWPDKSEGCNRSWPFNLEEIKDLLPEMRAYWPDVIHSFPNRSRFWKHEWEKHGTCAAQVDALNSQKKYFGRSLELYRELDLNSVLLKLGIKPSINYYQVADFKDALARVYGVIPKIQCLPPSQDEEVQTIGQIELCLTKQDQQLQNCTEPGEQPSPKQEVWLANGAAESRGLRVCEDGPVFYPPPKKTKH

SEQ ID NO: 72 - protein, 176 aa, Homo sapiens [sequence omitted]

SEQ ID NO: 73 - protein, 100 aa, Homo sapiens
MEEASEGGGNDRVRNLQSEVEGVKNIMTQNVERILARGENLEHLRNKTEDLEATSEHFKTTSQKVARKFWWKNVKMIVLICVIVFIIILFIVLFATGAFS

SEQ ID NO: 74 - protein, 537 aa, Homo sapiens
MAHCVTLVQLSISCDHLIDKDIGSKSDPLCVLLQDVGGGSWAELGRTERVRNCSSPEFSKTLQLEYRFETVQKLRFGIYDIDNKTPELRDDDFLGGAECSLGQIVSSQVLTLPLMLKPGKPAGRGTITVSAQELKDNRVVTMEVEARNLDKKDFLGKSDPFLEFFRQGDGKWHLVYRSEVIKNNLNPTWKRFSVPVQHFCGGNPSTPIQVQCSDYDSDGSHDLIGTFHTSLAQLQAVPAEFECIHPEKQQKKKSYKNSGTIRVKICRVETEYSFLDYVMGGCQINFTVGVDFTGSNGDPSSPDSLHYLSPTGVNEYLMALWSVGSVVQDYDSDKLFPAFGFGAQVPPDWQVSHEFALNFNPSNPYCAGIQGIVDAYRQALPQVRLYGPTNFAPIINHVARFAAQAAHQGTASQYFMLLLLTDGAVTDVEATREAVVRASNLPMSVIIVGVGGADFEAMEQLDADGGPLHTRSGQAAARDIVQFVPYRRFQNAPREALAQTVLAEVPTQLVSYFRAQGWAPLKPLPPSAKDPAQAPQA

SEQ ID NO: 75 - protein, 290 aa, Homo sapiens
MAPKRQSPLPPQKKKPRPPPALGPEETSASAGLPKKGEKEQQEAIEHIDEVQNEIDRLNEQASEEILKVEQKYNKLRQPFFQKRSELIAKIPNFWVTTFVNHPQVSALLGEEDEEALHYLTRVEVTEFEDIKSGYRIDFYFDENPYFENKVLSKEFHLNESGDPSSKSTEIKWKSGKDLTKRSSQTQNKASRKRQHEEPESFFTWFTDHSDAGADELGEVIKDDIWPNPLQYYLVPDMDDEEGEGEEDDDDDEEEEGLEDIDEEGDEDEGEEDEDDDEGEEGEEDEGEDD

SEQ ID NO: 76 - protein, 320 aa, Homo sapiens
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 77 - protein, 225 aa, Homo sapiens
MGFGDLKSPAGLQVLNDYLADKSYIEGYVPSQADVAVFEAVSSPPPADLCHALRWYNHIKSYEKEKASLPGVKKALGKYGPADVEDTTGSGATDSKDDDDIDLFGSDDEEESEEAKRLREERLAQYESKKAKKPALVAKSSILLDVKPWDDETDMAKLEECVRSIQADGLVWGSSKLVPVGYGIKKLQIQCVVEDDKVGTDMLEEQITAFEDYVQSMDVAAFNKI

SEQ ID NO: 78 - protein, 430 aa, Homo sapiens
MALLHSGRVLPGIAAAFHPGLAAAASARASSWWTHVEMGPPDPILGVTEAFKRDTNSKKMNLGVGAYRDDNGKPYVLPSVRKAEAQIAAKNLDKEYLPIGGLAEFCKASAELALGENSEVLKSGRFVTVQTISGTGALRIGASFLQRFFKFSRDVFLPKPTWGNHTPIFRDAGMQLQGYRYYDPKTCGFDFTGAVEDISKIPEQSVLLLHACAHNPTGVDPRPEQWKEIATVVKKRNLFAFFDMAYQGFASGDGDKDAWAVRHFIEQGINVCLCQSYAKNMGLYGERVGAFTMVCKDADEAKRVESQLKILIRPMYSNPPLNGARIAAAILNTPDLRKQWLQEVKVMADRIIGMRTQLVSNLKKEGSTHNWQHITDQIGMFCFTGLKPEQVERLIKEFSIYMTKDGRISVAGVTSSNVGYLAHAIHQVTK

SEQ ID NO: 79 - protein, 1101 aa, Homo sapiens
MSAKAISEQTGKELLYKFICTTSAIQNRFKYARVTPDTDWARLLQDHPWLLSQNLVVKPDQLIKRRGKLGLVGVNLTLDGVKSWLKPRLGQEATVGKATGFLKNFLIEPFVPHSQAEEFYVCIYATREGDYVLFHHEGGVDVGDVDAKAQKLLVGVDEKLNPEDIKKHLLVHAPEDKKEILASFISGLFNFYEDLYFTYLEINPLVVTKDGVYVLDLAAKVDATADYICKVKWGDIEFPPPFGREAYPEEAYIADLDAKSGASLKLTLLNPKGRIWTMVAGGGASVVYSDTICDLGGVNELANYGEYSGAPSEQQTYDYAKTILSLMTREKHPDGKILIIGGSIANFTNVAATFKGIVRAIRDYQGPLKEHEVTIFVRRGGPNYQEGLRVMGEVGKTTGIPIHVFGTETHMTAIVGMALGHRPIPNQPPTAAHTANFLLNASGSTSTPAPSRTASFSESRADEVAPAKKAKPAMPQDSVPSPRSLQGKSTTLFSRHTKAIVWGMQTRAVQGMLDFDYVCSRDEPSVAAMVYPFTGDHKQKFYWGHKEILIPVFKNMADAMRKHPEVDVLINFASLRSAYDSTMETMNYAQIRTIAIIAEGIPEALTRKLIKKADQKGVTIIGPATVGGIKPGCFKIGNTGGMLDNILASKLYRPGSVAYVSRSGGMSNELNNIISRTTDGVYEGVAIGGDRYPGSTFMDHVLRYQDTPGVKMIVVLGEIGGTEEYKICRGIKEGRLTKPIVCWCIGTCATMFSSEVQFGHAGACANQASETAVAKNQALKEAGVFVPRSFDELGEIIQSVYEDLVANGVIVPAQEVPPPTVPMDYSWARELGLIRKPASFMTSICDERGQELIYAGMPITEVFKEEMGIGGVLGLLWFQKRLPKYSCQFIEMCLMVTADHGPAVSGAHNTIICARAGKDLVSSLTSGLLTIGDRFGGALDAAAKMFSKAFDSGIIPMEFVNKMKKEGKLIMGIGHRVKSINNPDMRVQILKDYVRQHFPATPLLDYALEVEKITTSKKPNLILNVDGLIGVAFVDMLRNCGSFTREEADEYIDIGALNGIFVLGRSMGFIGHYLDQKRLKQGLYRHPWDDISYVLPEHMSM

SEQ ID NO: 80 - protein, 378 aa, Homo sapiens
MKDVPGFLQQSQNSGPGQPAVWHRLEELYTKNFMKTLSVNLNTGKSLSSVFHFENECIDARRCSKAGGFYFRVNPLSLVEIILHVVRQMTDPNVALTFLEKTREKVKSSDEAVILCKTAIGALKLNIGDLQVTKETIEDVEEMLNNLPGVTSVHSRFYDLSSKYYQTIGNHASYYKDALRFLGCVDIKDLPVSEQQERAFTLGLAGLLGEGVFNFGELLMHPVLESLRNTDRQWLIDTLYAFNSGNVERFQTLKTAWGQQPDLAANEAQLLRKIQLLCLMEMTFTRPANHRQLTFEEIAKSAKITVNEVELLVMKALSVGLVKGSIDEVDKRVHMTWVQPRVLDLQQIKGMKDRLEFWCTDVKSMEMLVEHQAHDILT

SEQ ID NO: 81 - protein, 181 aa, Homo sapiens
MGNIFGNLLKSLIGKKEMRILMVGLDAAGKTTILYKLKLGEIVTTIPTIGFNVETVEYKNISFTVWDVGGQDKIRPLWRHYFQNTQGLIFVVDSNDRERVNEAREELMRMLAEDELRDAVLLVFANKQDLPNAMNAAEITDKLGLHSLRHRNWYIQATCATSGDGLYEGLDWLANQLKNKK

SEQ ID NO: 82 - protein, 2639 aa, Homo sapiens
MSSSHSRAGQSAAGAAPGGGVDTRDAEMPATEKDLAEDAPWKKIQQNTFTRWCNEHLKCVSKRIANLQTDLSDGLRLIALLEVLSQKKMHRKHNQRPTFRQMQLENVSVALEFLDRESIKLVSIDSKAIVDGNLKLILGLIWTLILHYSISMPMWDEEEDEEAKKQTPKQRLLGWIQNKLPQLPITNFSRDWQSGRALGALVDSCAPGLCPDWDSWDASKPVTNAREAMQQADDWLGIPQVITPEEIVDPNVDEHSVMTYLSQFPKAKLKPGAPLRPKLNPKKARAYGPGIEPTGNMVKKRAEFTVETRSAGQGEVLVYVEDPAGHQEEAKVTANNDKNRTFSVWYVPEVTGTHKVTVLFAGQHIAKSPFEVYVDKSQGDASKVTAQGPGLEPSGNIANKTTYFEIFTAGAGTGEVEVVIQDPMGQKGTVEPQLEARGDSTYRCSYQPTMEGVHTVHVTFAGVPIPRSPYTVTVGQACNPSACRAVGRGLQPKGVRVKETADFKVYTKGAGSGELKVTVKGPKGEERVKQKDLGDGVYGFEYYPMVPGTYIVTITWGGQNIGRSPFEVKVGTECGNQKVRAWGPGLEGGVVGKSADFVVEAIGDDVGTLGFSVEGPSQAKIECDDKGDGSCDVRYWPQEAGEYAVHVLCNSEDIRLSPFMADIRDAPQDFHPDRVKARGPGLEKTGVAVNKPAEFTVDAKHGGKAPLRVQVQDNEGCPVEALVKDNGNGTYSCSYVPRKPVKHTAMVSWGGVSIPNSPFRVNVGAGSHPNKVKVYGPGVAKTGLKAHEPTYFTVDCAEAGQGDVSIGIKCAPGVVGPAEADIDFDIIRNDNDTFTVKYTPRGAGSYTIMVLFADQATPTSPIRVKVEPSHDASKVKAEGPGLSRTGVELGKPTHFTVNAKAAGKGKLDVQFSGLTKGDAVRDVDIIDHHDNTYTVKYTPVQQGPVGVNVTYGGDPIPKSPFSVAVSPSLDLSKIKVSGLGEKVDVGKDQEFTVKSKGAGGQGKVASKIVGPSGAAVPCKVEPGLGADNSVVRFLPREEGPYEVEVTYDGVPVPGSPFPLEAVAPTKPSKVKAFGPGLQGGSAGSPARFTIDTKGAGTGGLGLTVEGPCEAQLECLDNGDGTCSVSYVPTEPGDYNINILFADTHIPGSPFKAHVVPCFDASKVKCSGPGLERATAGEVGQFQVDCSSAGSAELTIEICSEAGLPAEVYIQDHGDGTHTITYIPLCPGAYTVTIKYGGQPVPNFPSKLQVEPAVDTSGVQCYGPGIEGQGVFREATTEFSVDARALTQTGGPHVKARVANPSGNLTETYVQDRGDGMYKVEYTPYEEGLHSVDVTYDGSPVPSSPFQVPVTEGCDPSRVRVHGPGIQSGTTNKPNKFTVETRGAGTGGLGLAVEGPSEAKMSCMDNKDGSCSVEYIPYEAGTYSLNVTYGGHQVPGSPFKVPVHDVTDASKVKCSGPGLSPGMVRANLPQSFQVDTSKAGVAPLQVKVQGPKGLVEPVDVVDNADGTQTVNYVPSREGPYSISVLYGDEEVPRSPFKVKVLPTHDASKVKASGPGLNTTGVPASLPVEFTIDAKDAGEGLLAVQITDPEGKPKKTHIQDNHDGTYTVAYVPDVTGRYTILIKYGGDEIPFSPYRVRAVPTGDASKCTVTGAGIGPTIQIGEETVITVDTKAAGKGKVTCTVCTPDGSEVDVDVVENEDGTFDIFYTAPQPGKYVICVRFGGEHVPNSPFQVTALAGDQPSVQPPLRSQQLAPQYTYAQGGQQTWAPERPLVGVNGLDVTSLRPFDLVIPFTIKKGEITGEVRMPSGKVAQPTITDNKDGTVTVRYAPSEAGLHEMDIRYDNMHIPGSPLQFYVDYVNCGHVTAYGPGLTHGVVNKPATFTVNTKDAGEGGLSLAIEGPSKAEISCTDNQDGTCSVSYLPVLPGDYSILVKYNEQHVPGSPFTARVTGDDSMRMSHLKVGSAADIPINISETDLSLLTATVVPPSGREEPCLLKRLRNGHVGISFVPKETGEHLVHVKKNGQHVASSPIPVVISQSEIGDASRVRVSGQGLHEGHTFEPAEFIIDTRDAGYGGLSLSIEGPSKVDINTEDLEDGTCRVTYCPTEPGNYIINIKFADQHVPGSPFSVKVTGEGRVKESITRRRRAPSVANVGSHCDLSLKIPEISIQDMTAQVTSPSGKTHEAEIVEGENHTYCIRFVPAEMGTHTVSVKYKGQHVPGSPFQFTVGPLGEGGAHKVRAGGPGLERAEAGVPAEFSIWTREAGAGGLAIAVEGPSKAEISFEDRKDGSCGVAYVVQEPGDYEVSVKFNEEHIPDSPFVVPVASPSGDARRLTVSSLQESGLKVNQPASFAVSLNGAKGAIDAKVHSPSGALEECYVTEIDQDKYAVRFIPRENGVYLIDVKFNGTHIPGSPFKIRVGEPGHGGDPGLVSAYGAGLEGGVTGNPAEFVVNTSNAGAGALSVTIDGPSKVKMDCQECPEGYRVTYTPMAPGSYLISIKYGGPYHIGGSPFKAKVTGPRLVSNHSLHETSSVFVDSLTKATCAPQHGAPGPGPADASKVVAKGLGLSKAYVGQKSSFTVDCSKAGNNMLLVGVHGPRTPCEEILVKHVGSRLYSVSYLLKDKGEYTLVVKWGDEHIPGSPYRVVVP

SEQ ID NO: 83 - protein, 272 aa, Homo sapiens
KKKKKKKKKNRVLFQAKENERYNTAHNVREKKKEVPAVPETLKKKRRNFAELKIKRLRKKFAQKMLRKARRKLIYEKAKHYHKEYRQMYRTEIRMARMARKAGNFYVPAEPKLAFVIRIRGINGVSPKVRKVLQLLRLRQIFNGTFVKLNKASINMLRIVEPYIAWGYPNLKSVNELIYKRGYGKINKKRIALTDNALIARSLGKYGIICMEDLIHEIYTVGKRFKEANNFLWPFKLSSPRGGMKKKTTHFVEGGDAGNREDQINRLIRRMN

SEQ ID NO: 84 - protein, 376 aa, Homo sapiens
MESYDVIANQPVVIDNGSGVIKAGFAGDQIPKYCFPNYVGRPKHVRVMAGALEGDIFIGPKAEEHRGLLSIRYPMEHGIVKDWNDMERIWQYVYSKDQLQTFSEEHPVLLTEAPLNPRKNRERAAEVFFETFNVPALFISMQAVLSLYATGRTTGVVLDSGDGVTHAVPIYEGFAMPHSIMRIDIAGRDVSRFLRLYLRKEGYDFHSSSEFEIVKAIKERACYLSINPQKDETLETEKAQYYLPDGSTIEIGPSRFRAPELLFRPDLIGEESEGIHEVLVFAIQKSDMDLRRTLFSNIVLSGGSTLFKGFGDRLLSEVKKLAPKDVKIRISAPQERLYSTWIGGSILASLDTFKKMWVSKKEYEEDGARSIHRKTF

SEQ ID NO: 85 - protein, 102 aa, Homo sapiens
MAGQAFRKFLPLFDRVLVERSAAETVTKGGIMLPEKSQGKVLQATVVAVGSGSKGKGGEIQPVSVKVGDKVLLPEYGGTKVVLDDKDYFLFRDGDILGKYVD

SEQ ID NO: 86 - protein, 118 aa, Homo sapiens
MSRQANRGTESKKMSSELFTLTYGALVTQLCKDYENDEDVNKQLDKMGFNIGVRLIEDFLARSNVGRCHDFRETADVIAKERKMFLGKQNKTKRQAKLYDAVKERGAASCTDASPLLS

SEQ ID NO: 87 - protein, 130 aa, Homo sapiens [sequence omitted]

SEQ ID NO: 88 - protein, 341 aa, Homo sapiens
MPFPFGKSHKSPADIVKNLKESMAVLEKQDISDKKAEKATEEVSKNLVAMKEILYGTNEKEPQTEAVAQLAQELYNSGLLSTLVADLQLIDFEGKKDVAQIFNNILRRQIGTRTPTVEYICTQQNILFMLLKGYESPEIALNCGIMLRECIRHEPLAKIILWSEQFYDFFRYVEMSTFDIASDAFATFKDLLTRHKLLSAEFLEQHYDRFFSEYEKLLHSENYVTKRQSLKLLGELLLDRHNFTIMTKYISKPENLKLMMNLLRDKSRNIQFEAFHVFKVFVANPNKTQPILDILLKNQAKLIEFLSKFQNDRTEDEQFNDEKTYLVKQIRDLKRPAQQEA

SEQ ID NO: 89 - protein, 763 aa, Homo sapiens
MVACRAIGILSRFSAFRILRSRGYICRNFTGSSALLTRTHINYGVKGDVAVVRINSPNSKVNTLSKELHSEFSEVMNEIWASDQIRSAVLISSKPGCFIAGADINMLAACKTLQEVTQLSQEAQRIVEKLEKSTKPIVAAINGSCLGGGLEVAISCQYRIATKDRKTVLGTPEVLLGALPGAGGTQRLPKMVGVPAALDMMLTGRSIRADRAKKMGLVDQLVEPLGPGLKPPEERTIEYLEEVAITFAKGLADKKISPKRDKGLVEKLTAYAMTIPFVRQQVYKKVEEKVRKQTKGLYPAPLKIIDVVKTGIEQGSDAGYLCESQKFGELVMTKESKALMGLYHGQVLCKKNKFGAPQKDVKHLAILGAGLMGAGIAQVSVDKGLKTILKDATLTALDRGQQQVFKGLNDKVKKKALTSFERDSIFSNLTGQLDYQGFEKADMVIEAVFEDLSLKHRVLKEVEAVIPDHCIFASNTSALPISEIAAVSKRPEKVIGMHYFSPVDKMQLLEIITTEKTSKDTSASAVAVGLKQGKVIIVVKDGPGFYTTRCLAPMMSEVIRILQEGVDPKKLDSLTTSFGFPVGAATLVDEVGVDVAKHVAEDLGKVFGERFGGGNPELLTQMVSKGFLGRKSGKGFYIYQEGVKRKDLNSDMDSILASLKLPPKSEVSSDEDIQFRLVTRFVNEAVMCLQEGILATPAEGDIGAVFGLGFPPCLGGPFRFVDLYGAQKIVDRLKKYEAAYGKQFTPCQLLADHANSPNKKFYQ

SEQ ID NO: 90 - protein, 999 aa, Homo sapiens
MADKVRRQRPRRRVCWALVAVLLADLLALSDTLAVMSVDLGSESMKVAIVKPGVPMEIVLNKESRRKTPVIVTLKENERFFGDSAASMAIKNPKATLRYFQHLLGKQADNPHVALYQARFPEHELTFDPQRQTVHFQISSQLQFSPEEVLGMVLNYSRSLAEDFAEQPIKDAVITVPVFFNQAERRAVLQAARMAGLKVLQLINDNTATALSYGVFRRKDINTTAQNIMFYDMGSGSTVCTIVTYQMVKTKEAGMQPQLQIRGVGFDRTLGGLEMELRLRERLAGLFNEQRKGQRAKDVRENPRAMAKLLREANRLKTVLSANADHMAQIEGLMDDVDFKAKVTRVEFEELCADLFERVPGPVQQALQSAEMSLDEIEQVILVGGATRVPRVQEVLLKAVGKEELGKNINADEAAAMGAVYQAAALSKAFKVKPFVVRDAVVYPILVEFTREVEEEPGIHSLKHNKRVLFSRMGPYPQRKVITFNRYSHDFNFHINYGDLGFLGPEDLRVFGSQNLTTVKLKGVGDSFKKYPDYESKGIKAHFNLDESGVLSLDRVESVFETLVEDSAEEESTLTKLGNTISSLFGGGTTPDAKENGTDTVQEEEESPAEGSKDEPGEQVELKEEAEAPVEDGSQPPPPEPKGDATPEGEKATEKENGDKSEAQKPSEKAEAGPEGVAPAPEGEKKQKPARKRRMVEEIGVELVVLDLPDLPEDKLAQSVQKLQDLTLRDLEKQEREKAANSLEAFIFETQDKLYQPEYQEVSTEEQREEISGKLSAASTWLEDEGVGATTVMLKEKLAELRKLCQGLFFRVEERKKWPERLSALDNLLNHSSMFLKGARLIPEMDQIFTEVEMTTLEKVINETWAWKNATLAEQAKLPATEKPVLLSKDIEAKMMALDREVQYLLNKAKFTKPRPRPKDKNGTRAEPPLNASASDQGEKVIPPAGQTEDAEPISEPEKVETGSEPGDTEPLELGGPGAEPEQKEQSTGQKRPLKNDEL

SEQ ID NO: 91 - protein, 191 aa, Homo sapiens
MAAYSYRPGPGAGPGPAAGAALPDQSFLWNVFQRVDKDRSGVISDTELQQALSNGTWTPFNPVTVRSIISMFDRENKAGVNFSEFTGVWKYITDWQNVFRTYDRDNSGMIDKNELKQALSGFGYRLSDQFHDILIRKFDRQGRGQIAFDDFIQGCIVLQRLTDIFRRYDTDQDGWIQVSYEQYLSMVFSIV

SEQ ID NO: 92 - protein, 216 aa, Homo sapiens
MAAQGEPQVQFKLVLVGDGGTGKTTFVKRHLTGEFEKKYVATLGVEVHPLVFHTNRGPIKFNVWDTAGQEKFGGLRDGYYIQAQCAIIMFDVTSRVTYKNVPNWHRDLVRVCENIPIVLCGNKVDIKDRKVKAKSIVFHRKKNLQYYDISAKSNYNFEKPFLWLARKLIGDPNLEFVAMPALAPPEVVMDPALAAQYEHDLEVAQTTALPDEDDDL

SEQ ID NO: 93 - protein, 135 aa, Homo sapiens
MACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFD

SEQ ID NO: 94 - protein, 261 aa, Homo sapiens
MFEARLVQGSILKKVLEALKDLINEACWDISSSGVNLQSMDSSHVSLVQLTLRSEGFDTYRCDRNLAMGVNLTSMSKILKCAGNEDIITLRAEDNADTLALVFEAPNQEKVSDYEMKLMDLDVEQLGIPEQEYSCVVKMPSGEFARICRDLSHIGDAVVISCAKDGVKFSASGELGNGNIKLSQTSNVDKEEEAVTIEMNEPVQLTFALRYLNFFTKATPLSSTVTLSMSADVPLVVEYKIADMGHLKYYLAPKIEDEEGS

SEQ ID NO: 95 - protein, 226 aa, Homo sapiens
MSETAPAETATPAPVEKSPAKKKATKKAAGAGAAKRKATGPPVSELITKAVAASKERNGLSLAALKKALAAGGYDVEKNNSRIKLGLKSLVSKGTLVQTKGTGASGSFKLNKKAASGEAKPKAKKAGAAKAKKPAGATPKKAKKAAGAKKAVKKTPKKAKKPAAAGVKKVAKSPKKAKAAAKPKKATKSPAKPKAVKPKAAKPKAAKPKAAKPKAAKAKKAAAKKK

SEQ ID NO: 96 - protein, 286 aa, Homo sapiens
MAEDGEEAEFHFAALYISGQWPRLRADTDLQRLGSSAMAPSRKFFVGGNWKMNGRKQSLGELIGTLNAAKVPADTEVVCAPPTAYIDFARQKLDPKIAVAAQNCYKVTNGAFTGEISPGMIKDCGATWVVLGHSERRHVFGESDELIGQKVAHALAEGLGVIACIGEKLDEREAGITEKVVFEQTKVIADNVKDWSKVVLAYEPVWAIGTGKTATPQQAQEVHEKLRGWLKSNVSDAVAQSTRIIYGGSVTGATCKELASQPDVDGFLVGGASLKPEFVDIINAKQ

SEQ ID NO: 97 - protein, 283 aa, Homo sapiens
MAVPPTYADLGKSARDVFTKGYGFGLIKLDLKTKSENGLEFTSSGSANTETTKVTGSLETKYRWTEYGLTFTEKWNTDNTLGTEITVEDQLARGLKLTFDSSFSPNTGKKNAKIKTGYKREHINLGCDMDFDIAGPSIRGALVLGYEGWLAGYQMNFETAKSRVTQSNFAVGYKTDEFQLHTNVNDGTEFGGSIYQKVNKKLETAVNLAWTAGNSNTRFGIAAKYQIDPDACFSAKVNNSSLIGLGYTQTLKPGIKLTLSALLDGKNVNAGGHKLGLGLEFQA

SEQ ID NO: 98 - protein, 105 aa, Homo sapiens
MVKQIESKTAFQEALDAAGDKLVVVDFSATWCGPCKMIKPFFHSLSEKYSNVIFLEVDVDDCQDVASECEVKCMPTFQFFKKGQKVGEFSGANKEKLEATINELV

SEQ ID NO: 99 - protein, 199 aa, Homo sapiens
MSSGNAKIGHPAPNFKATAVMPDGQFKDISLSDYKGKYVVFFFYPLDFTFVCPTEIIAFSDRAEEFKKLNCQVIGASVDSHFCHLAWVNTPKKQGGLGPMNIPLVSDPKRTIAQDYGVLKADEGISFRGLFIIDDKGILRQITVNDLPVGRSVDETLRLVQAFQFTDKHGEVCPAGWKPGSDTIKPDVQKSKEYFSKQK

SEQ ID NO: 100 - protein, 105 aa, Homo sapiens
MAKISSPTETERCIESLIAVFQKYAGKDGYNYTLSKTEFLSFMNTELAAFTKNQKDPGVLDRMMKKLDTNSDGQLDFSEFLNLIGGLAMACHDSFLKAVPSQKRT

SEQ ID NO: 101 - protein, 445 aa, Homo sapiens
MREIVHLQAGQCGNQIGAKFWEVISDEHGIDPTGTYHGDSDLQLERINVYYNEATGGKYVPRAVLVDLEPGTMDSVRSGPFGQIFRPDNFVFGQSGAGNNWAKGHYTEGAELVDSVLDVVRKEAESCDCLQGFQLTHSLGGGTGSGMGTLLISKIREEYPDRIMNTFSVVPSPKVSDTVVEPYNATLSVHQLVENTDETYCIDNEALYDICFRTLKLTTPTYGDLNHLVSATMSGVTTCLRFPGQLNADLRKLAVNMVPFPRLHFFMPGFAPLTSRGSQQYRALTVPELTQQMFDAKNMMAACDPRHGRYLTVAAVFRGRMSMKEVDEQMLNVQNKNSSYFVEWIPNNVKTAVCDIPPRGLKMSATFIGNSTAIQELFKRISEQFTAMFRRKAFLHWYTGEGMDEMEFTEAESNMNDLVSEYQQYQDATAEEEGEFEEEAEEEVA

SEQ ID NO: 102 - protein, 463 aa, Homo sapiens
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEGLQLPSPTATSQLPLESDAVECLNYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDYDDMSPRRGPPPPPPGRGGRGGSRARNLPLPPPPPPRGGDLMAYDRRGRPGDRYDGMVGFSADETWDSAIDTWSPSEWQMAYEPQGGSGYDYSYAGGRGSYGDLGGPIITTQVTIPKDLAGSIIGKGGQRIKQIRHESGASIKIDEPLEGSEDRIITITGTQDQIQNAQYLLQNSVKQYSGKFF

SEQ ID NO: 103 - protein, 461 aa, Homo sapiens
MSRQVVRSSKFRHVFGQPAKADQCYEDVRVSQTTWDSGFCAVNPKFVALICEASGGGAFLVLPLGKTGRVDKNAPTVCGHTAPVLDIAWCPHNDNVIASGSEDCTVMVWEIPDGGLMLPLREPVVTLEGHTKRVGIVAWHTTAQNVLLSAGCDNVIMVWDVGTGAAMLTLGPEVHPDTIYSVDWSRDGGLICTSCRDKRVRIIEPRKGTVVAEKDRPHEGTRPVRAVFVSEGKILTTGFSRMSERQVALWDTKHLEEPLSLQELDTSSGVLLPFFDPDTNIVYLCGKGDSSIRYFEITSEAPFLHYLSMFSSKESQRGMGYMPKRGLEVNKCEIARFYKLHERRCEPIAMTVPRKSDLFQEDLYPPTAGPDPALTAEEWLGGRDAGPLLISLKDGYVPPKSRELRVNRGLDTGRRRAAPEASGTPSSDAVSRLEEEMRKLQATVQELQKRLDRLEETVQAK

SEQ ID NO: 104 - protein, 724 aa, Homo sapiens
MPEEVHHGEEEVETFAFQAEIAQLMSLIINTFYSNKEIFLRELISNASDALDKIRYESLTDPSKLDSGKELKIDIIPNPQERTLTLVDTGIGMTKADLINNLGTIAKSGTKAFMEALQAGADISMIGQFGVGFYSAYLVAEKVVVITKHNDDEQYAWESSAGGSFTVRADHGEPIGRGTKVILHLKEDQTEYLEERRVKEVVKKHSQFIGYPITLYLEKEREKEISDDEAEEEKGEKEEEDKDDEEKPKIEDVGSDEEDDSGKDKKKKTKKIKEKYIDQEELNKTKPIWTRNPDDITQEEYGEFYKSLTNDWEDHLAVKHFSVEGQLEFRALLFIPRRAPFDLFENKKKKNNIKLYVRRVFIMDSCDELIPEYLNFIRGVVDSEDLPLNISREMLQQSKILKVIRKNIVKKCLELFSELAEDKENYKKFYEAFSKNLKLGIHEDSTNRRRLSELLRYHTSQSGDEMTSLSEYVSRMKETQKSIYYITGESKEQVANSAFVERVRKRGFEVVYMTEPIDEYCVQQLKEFDGKSLVSVTKEGLELPEDEEEKKKMEESKAKFENLCKLMKEILDKKVEKVTISNRLVSSPCCIVTSTYGWTANMERIMKAQALRDNSTMGYMMAKKHLEINPDHPIVETLRQKAEADKNDKAVKDLVVLLFETALLSSGFSLEDPQTHSNRIYRMIKLGLGIDEDEVAAEEPNAAVPDEIPPLEGDEDASRMEEVD

SEQ ID NO: 105 - protein, 573 aa, Homo sapiens
MLRLPTVFRQMRPVSRVLAPHLTRAYAKDVKFGADARALMLQGVDLLADAVAVTMGPKGRTVIIEQSWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLTTAEVVVTEIPKEEKDPGMGAMGGMGGGMGGGMF

SEQ ID NO: 106 - protein, 286 aa, Homo sapiens
MADFDDRVSDEEKVRIAAKFITHAPPGEFNEVFNDVRLLLNNDNLLREGAAHAFAQYNMDQFTPVKIEGYEDQVLITEHGDLGNSRFLDPRNKISFKFDHLRKEASDPQPEEADGGLKSWRESCDSALRAYVKDHYSNGFCTVYAKTIDGQQTIIACIESHQFQPKNFWNGRWRSEWKFTITPPTAQVVGVLKIQVHYYEDGNVQLVSHKDVQDSLTVSNEAQTAKEFIKIIENAENEYQTAISENYQTMSDTTFKALRRQLPVTRTKIDWNKILSYKIGKEMQNA

SEQ ID NO: 107 - protein, 103 aa, Homo sapiens [sequence omitted]

SEQ ID NO: 108 - protein, 147 aa, Homo sapiens
MVHFTAEEKAAVTSLWSKMNVEEAGGEALGRLLVVYPWTQRFFDSFGNLSSPSAILGNPKVKAHGKKVLTSFGDAIKNMDNLKPAFAKLSELHCDKLHVDPENFKLLGNVMVIILATHFGKEFTPEVQAAWQKLVSAVAIALAHKYH

SEQ ID NO: 109 - protein, 142 aa, Homo sapiens
MFACAKLACTPSLIRAGSRVAYRPISASVLSRPEASRTGEGSTVFNGAQNGVSQLIQREFQTSAISRDIDTAAKFIGAGAATVGVAGSGAGIGTVFGSLIIGYARNPSLKQQLFSYAILGFALSEAMGLFCLMVAFLILFAM

SEQ ID NO: 110 - protein, 854 aa, Homo sapiens
MPPCSGGDGSTPPGPSLRDRDCPAQSAEYPRDRLDPRPGSPSEASSPPFLRSRAPVNWYQEKAQVFLWHLMVSGSTTLLCLWKQPFHVSAFPVTASLAFRQSQGAGQHLYKDLQPFILLRLLMPEETQTQDQPMEEEEVETFAFQAEIAQLMSLIINTFYSNKEIFLRELISNSSDALDKIRYESLTDPSKLDSGKELHINLIPNKQDRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPITLFVEKERDKEVSDDEAEEKEDKEEEKEKEEKESEDKPEIEDVGSDEEEEKKDGDKKKKKKIKEKYIDQEELNKTKPIWTRNPDDITNEEYGEFYKSLTNDWEDHLAVKHFSVEGQLEFRALLFVPRRAPFDLFENRKKKNNIKLYVRRVFIMDNCEELIPEYLNFIRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHIYYITGETKDQVANSAFVERLRKHGLEVIYMIEPIDEYCVQQLKEFEGKTLVSVTKEGLELPEDEEEKKKQEEKKTKFENLCKIMKDILEKKVEKVVVSNRLVTSPCCIVTSTYGWTANMERIMKAQALRDNSTMGYMAAKKHLEINPDHSIIETLRQKAEADKNDKSVKDLVILLYETALLSSGFSLEDPQTHANRIYRMIKLGLGIDEDDPTADDTSAAVTEEMPPLEGDDDTSRMEEVD

SEQ ID NO: 111 - protein, 142 aa, Homo sapiens
MVLSPADKTNVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 112 - protein, 858 aa, Homo sapiens
MVNFTVDQIRAIMDKKANIRNMSVIAHVDHGKSTLTDSLVCKAGIIASARAGETRFTDTRKDEQERCITIKSTAISLFYELSENDLNFIKQSKDGAGFLINLIDSPGHVDFSSEVTAALRVTDGALVVVDCVSGVCVQTETVLRQAIAERIKPVLMMNKMDRALLELQLEPEELYQTFQRIVENVNVIISTYGEGESGPMGNIMIDPVLGTVGFGSGLHGWAFTLKQFAEMYVAKFAAKGEGQLGPAERAKKVEDMMKKLWGDRYFDPANGKFSKSATSPEGKKLPRTFCQLILDPIFKVFDAIMNFKKEETAKLIEKLDIKLDSEDKDKEGKPLLKAVMRRWLPAGDALLQMITIHLPSPVTAQKYRCELLYEGPPDDEAAMGIKSCDPKGPLMMYISKMVPTSDKGRFYAFGRVFSGLVSTGLKVRIMGPNYTPGKKEDLYLKPIQRTILMMGRYVEPIEDVPCGNIVGLVGVDQFLVKTGTITTFEHAHNMRVMKFSVSPVVRVAVEAKNPADLPKLVEGLKRLAKSDPMVQCIIEESGEHIIAGAGELHLEICLKDLEEDHACIPIKKSDPVVSYRETVSEESNVLCLSKSPNKHNRLYMKARPFPDGLAEDIDKGEVSARQELKQRARYLAEKYEWDVAEARKIWCFGPDGTGPNILTDITKGVQYLNEIKDSVVAGFQWATKEGALCEENMRGVRFDVHDVTLHADAIHRGGGQIIPTARRCLYASVLTAQPRLMEPIYLVEIQCPEQVVGGIYGVLNRKRGHVFEESQVAGTPMFVVKAYLPVNESFGFTADLRSNTGGQAFPQCVFDHWQILPGDPFDNSSRPSQVVAETRKRKGLKEGIPALDNFLDKL

SEQ ID NO: 113 - protein, 136 aa, Homo sapiens [sequence omitted]

SEQ ID NO: 114 - protein, 199 aa, Homo sapiens
MANRGPAYGLSREVQQKIEKQYDADLEQILIQWITTQCRKDVGRPQPGRENFQNWLKDGTVLCELINALYPEGQAPVKKIQASTMAFKQMEQISQFLQAAERYGINTTDIFQTVDLWEGKNMACVQRTLMNLGGLAVARDDGLFSGDPNWFPKKSKENPRNFSDNQLQEGKNVIGLQMGTNRGASQAGMTGYGMPRQIL

SEQ ID NO: 115 - protein, 529 aa, Homo sapiens
MLGFVGRVAAAPASGALRRLTPSASLPPAQLLLRAAPTAVHPVRDYAAQTSPSPKAGAATGRIVAVIGAVVDVQFDEGLPPILNALEVQGRETRLVLEVAQHLGESTVRTIAMDGTEGLVRGQKVLDSGAPIKIPVGPETLGRIMNVIGEPIDERGPIKTKQFAPIHAEAPEFMEMSVEQEILVTGIKVVDLLAPYAKGGKIGLFGGAGVGKTVLIMELINNVAKAHGGYSVFAGVGERTREGNDLYHEMIESGVINLKDATSKVALVYGQMNEPPGARARVALTGLTVAEYFRDQEGQDVLLFIDNIFRFTQAGSEVSALLGRIPSAVGYQPTLATDMGTMQERITTTKKGSITSVQAIYVPADDLTDPAPATTFAHLDATTVLSRAIAELGIYPAVDPLDSTSRIMDPNIVGSEHYDVARGVQKILQDYKSLQDIIAILGMDELSEEDKLTVSRARKIQRFLSQPFQVAEVFTGHMGKLVPLKETIKGFQQILAGEYDHLPEQAFYMVGPIEEAVAKADKLAEEHSS

SEQ ID NO: 116 - protein, 364 aa, Homo sapiens
MPYQYPALTPEQKKELSDIAHRIVAPGKGILAADESTGSIAKRLQSIGTENTEENRRFYRQLLLTADDRVNPCIGGVILFHETLYQKADDGRPFPQVIKSKGGVVGIKVDKGVVPLAGTNGETTTQGLDGLSERCAQYKKDGADFAKWRCVLKIGEHTPSALAIMENANVLARYASICQQNGIVPIVEPEILPDGDHDLKRCQYVTEKVLAAVYKALSDHHIYLEGTLLKPNMVTPGHACTQKFSHEEIAMATVTALRRTVPPAVTGITFLSGGQSEEEASINLNAINKCPLLKPWALTFSYGRALQASALKAWGGKKENLKAAQEEYVKRALANSLACQGKYTPSGQAGAAASESLFVSNHAY

SEQ ID NO: 117 - protein, 335 aa, Homo sapiens
MGKVKVGVNGFGRIGRLVTRAAFNSGKVDIVAINDPFIDLNYMVYMFQYDSTHGKFHGTVKAENGKLVINGNPITIFQERDPSKIKWGDAGAEYVVESTGVFTTMEKAGAHLQGGAKRVIISAPSADAPMFVMGVNHEKYDNSLKIISNASCTTNCLAPLAKVIHDNFGIVEGLMTTVHAITATQKTVDGPSGKLWRDGRGALQNIIPASTGAAKAVGKVIPELNGKLTGMAFRVPTANVSVVDLTCRLEKPAKYDDIKKVVKQASEGPLKGILGYTEHQVVSSDFNSDTHSSTFDAGAGIALNDHFVKLISWYDNEFGYSNRVVDLMAHMASKE

SEQ ID NO: 118 - protein, 377 aa, Homo sapiens
MCEEEDSTALVCDNGSGLCKAGFAGDDAPRAVFPSIVGRPRHQGVMVGMGQKDSYVGDEAQSKRGILTLKYPIEHGIITNWDDMEKIWHHSFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVLSGGTTMYPGIADRMQKEITALAPSTMKIKIIAPPERKYSVWIGGSILASLSTFQQMWISKQEYDEAGPSIVHRKCF

SEQ ID NO: 119 - protein, 108 aa, Homo sapiens
MKALCLLLLPVLGLLVSSKTLCSMEEAINERIQEVAGSLIFRAISSIGLECQSVTSRGDLATCPRGFAVTGCTCGSACGSWDVRAETTCHCQCAGMDWTGARCCRVQP

SEQ ID NO: 120 - protein, 441 aa, Homo sapiens
MGKEKTHINIVVIGHVDSGKSTTTGHLIYKCGGIDKRTIEKFEKEAAEMGKGSFKYAWVLDKLKAERERGITIDISLWKFETSKYYVTIIDAPGHRDFIKNMITGTSQADCAVLIVAAGVGEFEAGISKNGQTREHALLAYSQKRYEEIVKEVSTYIKKIGYNPDTVAFVPISGWNGDNMLEPSANMPWFKGWKVTRKDGNASGTTLLEALDCILPPTRPTDKPLRLPLQDVYKIGGIGTVPVGRVETGVLKPGMVVTFAPVNVTTEVKSVEMHHEALSEALPGDNVGFNVKNVSVKDVRRGNVAGDSKNDPPMEAAGFTAQVIILNHPGQISAGYAPVLDCHTAHIACKFAELKEKIDRRSGKKLEDGPKFLKSGDAAIVDMVPGKPMCVESFSDYPPLGRFAVRDMRQTVAVGVIKAVDKKAAGAGKVTKSAQKAQKAK

SEQ ID NO: 121 - protein, 463 aa, Homo sapiens
MGKEKTHINIVVIGHVDSGKSTTTGHLIYKCGGIDKRTIEKFEKEAAEMGKGSFKYAWVLDKLKAERERGITIDISLWKFETTKYYITIIDAPGHRDFIKNMITGTSQADCAVLIVAAGVGEFEAGISKNGQTREHALLAYTLGVKQLIVGVNKMDSTEPAYSEKRYDEIVKEVSAYIKKIGYNPATVPFVPISGWHGDNMLEPSPNMPWFKGWKVERKEGNASGVSLLEALDTILPPTRPTDKPLRLPLQDVYKIGGIGTVPVGRVETGILRPGMVVTFAPVNITTEVKSVEMHHEALSEALPGDNVGFNVKNVSVKDIRRGNVCGDSKSDPPQEAAQFTSQVIILNHPGQISAGYSPVIDCHTAHIACKFAELKEKIDRRSGKKLEDNPKSLKSGDAAIVEMVPGKPMCVESFSQYPPLGRFAVRDMRQTVAVGVIKNVEKKSGGAGKVTKSAQKAQKAGK

SEQ ID NO: 122 - protein, 153 aa, Homo sapiens
MADEIDFTTGDAGASSTYPMQCSALRKNGFVVLKGRPCKIVEMSTSKTGKHGHAKVHLVGIDIFTGKKYEDICPSTHNMDVPNIKRNDYQLICIQDGYLSLLTETGEVREDLKLPEGELGKEIEGKYNAGEDVQVSVMCAMSEEYAVAIKPCK

SEQ ID NO: 123 - protein, 142 aa, Homo sapiens
MSLTKTERTIIVSMWAKISTQADTIGTETLERLFLSHPQTKTYFPHFDLHPGSAQLRAHGSKVVAAVGDAVKSIDDIGGALSKLSELHAYILRVDPVNFKLLSHCLLVTLAARFPADFTAEAHAAWDKFLSVVSSVLTEKYR

SEQ ID NO: 124 - protein, 343 aa, Homo sapiens
MPEETQTQDQPMEEEEVETFAFQAEIAQLMSLIINTFYSNKEIFLRELISNSSDALDKIWYESLTDPSKLDSGKELHINLIPNKQDQTLTIVDTGIGMTKADLINNLGTIAKSGTKAFMEALQAGADISMIGQFGVSFYSAYLVAEKVTVITKHNDDEQYAWESSAGGSFTVRTDTGERMGRGTKVILHLKEDQTEYLEEQRIKEIVKKHSQLIGYPITLFVEKECDKEVSDDETEEKEDKEEEKEKEEKESKDKPEIEDVGSDEEEEKKDGDKKKKKTKEKYIDQEELNKTKPIWTRNPDDITNEEYGEFCKNLTNDWEDHLAVKHFSVEGQLEFRALLFVP

SEQ ID NO: 125 - protein, 603 aa, Homo sapiens
MVKLAKAGKNQGDPKKMAPPPKEVEEDSEDEEMSEDEEDDEDDDDDEEDDSEEEAMETTPAKGKKAAKVVPVKAKGAKNGKNAKKEDSDEEEDDDSEEDEEDDEDEDEDEDEIEPAAMKAAAAAPASEDEDDEDDEDDEDDDDDEDDEDDDEDDEEEEEEEEEEPVKEAPGKRKKEMAKQKAAPEAKKQKVEGTEPTTAFNLFVGNLNFNKSAPELKTGISDVFAKNDLAVVDVRIGMTRKFGYVDFESAEDLEKALELTGLKVFGNEIKLEKPKGKDSKKERDARTLLAKNLPYKVTQDELKEVFEDAAEIRLVSKDGNSKGIAYIEFKTEADAEKTFEEKQGTEIDGRSISLYYTGEKGQNQDYRGGKNSTWSGESKTLVLSNLSYSATEETLQEVFEKATFIKVPQNQNGKSKGYAFIEFASFEDAKEALNSCNKREIEGRAIRLELQGPRGSPNARSQPSKTLFVKGLSEDTTEETLKESFDGSVRARIVTDRETGSSKGFGFVDFNSEEDAKAAKEAMEDGEIDGNKVTLDWAKPKGEGGFGGRGGGRGGFGGRGGGRGGRGGFGGRGRGGFGGRGGFRGGRGGGGDHKPQGKKTKFE

SEQ ID NO: 126 - protein, 265 aa, Homo sapiens
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEEEEDVKLLSISGKRSAPGGGSKVPQKKVKLAADEDDDDDDEEDDDEDDDDDDFDDEEAEEKAPVKKGQESFKKQEKTPKTPKGPSSVEDIKAKMQASIEKGGSLPKVEAKFINYVKNCFRMTDQEAIQDLWQWRKSL

SEQ ID NO: 127 - protein, 164 aa, Homo sapiens
MVNSVVFFDITVDGKPLGRISIKLFADKILKTAENFRALSTGEKGFRYKGSCFHRIIPGFMCQGGDFTRHNGTGDKSIYGEKFDDENLIRKHTGSGILSMANAGPNTNGSQFFICAAKTEWLDGKHVAFGKVKERVNIVEAMEHFGYRNSKTSKKITIADCGQF

SEQ ID NO: 128 - protein, 249 aa, Homo sapiens
MAPSRKFFVGGNWKMNGRKQSLGELIGTLNAAKVPADTEVVCAPPTAYIDFARQKLDPKIAVAAQNCYKVTNGAFTGEISPGMIKDCGATWVVLGHSERRHVFGESDELIGQKVAHALAEGLGVIACIGEKLDEREAGITEKVVFEQTKVIADNVKDWSKVVLAYEPVWAIGTGKTATPQQAQEVHEKLRGWLKSNVSDAVAQSTRIIYGGSVTGATCKELASQPDVDGFLVGGASLKPEFVDIINAKQ

SEQ ID NO: 129 - protein, 407 aa, Homo sapiens
MSHRKFSAPRHGHLGFLPHKRSHRHRGKVKTWPRDDPSQPVHLTAFLGYKAGMTHTLREVHRPGLKISKREEVEAVTIVETPPLVVVGVVGYVATPRGLRSFKTIFAEHLSDECRRRFYKDWHKSKKKAFTKACKRWRDTDGKKQLQKDFAAMKKYCKVIRVIVHTQMKLLPFRQKKAHIMEIQLNGGTVAEKVAWAQARLEKQVPVHSVFSQSEVIDVIAVTKGRGVKGVTSRWHTKKLPRKTHKGLRKVACIGAWHPARVGCSIARAGQKGYHHRTELNKKIFRIGRGPHMEDGKLVKNNASTSYDVTAKSITPLGGFPHYGEVNNDFVMLKGCIAGTKKRVITLRKSLLVHHSRQAVENIELKFIDTTSKFGHGRFQTAQEKRAFMGPQKKHLEKETPETSGDL

SEQ ID NO: 130 - protein, 326 aa, Homo sapiens
MRECISIHVGQAGVQIGNACWELYCLEHGIQPDGQMPSDKTIGGGDDSFNTFFSETGAGKHVPRAVFVDLEPTVIDEVRTGTYRQLFHPEQLITGKEDAANNYARGHYTIGKEIIDLVLDRIRKLKSFFATKPRIHFPLATYAPVISAEKAYHEQLTVAEITNACFEPANQMVKCDPRHGKYMACCLLYRGDVVPKDVNAAIATIKTKRTIQFVDWCPTGFKVGINYQPPTVVPGGDLAKVQRAVCMLSNTTAVAEAWARLDHKFDLMPLRRIMRRLEQIVLTERMRVKSINLCAVLLHSFVLELSYFCSVNVYCRKLLIKLKFPF

SEQ ID NO: 131 - protein, 11 aa, Homo sapiens
AGQAVDDFIEK

SEQ ID NO: 132 - protein, 17 aa, Homo sapiens
GILFVGSGVSGGEEGAR

SEQ ID NO: 133 - protein, 10 aa, Homo sapiens
VDDFLANEAK

SEQ ID NO: 134 - protein, 8 aa, Homo sapiens
GTLDPVEK

SEQ ID NO: 135 - protein, 18 aa, Homo sapiens
TVTNAVVTVPAYFNDSQR

SEQ ID NO: 136 - protein, 7 aa, Homo sapiens
VFFASWR

SEQ ID NO: 137 - protein, 12 aa, Homo sapiens
VVLEGGIDPILR

SEQ ID NO: 138 - protein, 8 aa, Homo sapiens
AVSNEIVR

SEQ ID NO: 139 - protein, 12 aa, Homo sapiens
FPTDQLTPDQER

SEQ ID NO: 140 - protein, 8 aa, Homo sapiens
ELPSFLGK

SEQ ID NO: 141 - protein, 11 aa, Homo sapiens
ALNSIIDVYHK

SEQ ID NO: 142 - protein, 13 aa, Homo sapiens
LGHPDTLNQGEFK

SEQ ID NO: 143 - protein, 15 aa, Homo sapiens
NIETIINTFHQYSVK

SEQ ID NO: 144 - protein, 14 aa, Homo sapiens
GDGPVQGIINFEQK

SEQ ID NO: 145 - protein, 7 aa, Homo sapiens
ETIEQEK

SEQ ID NO: 146 - protein, 6 aa, Homo sapiens
NPLPSK

SEQ ID NO: 147 - protein, 10 aa, Homo sapiens
VQPNEAVYTK

SEQ ID NO: 148 - protein, 14 aa, Homo sapiens
TYNFLPEFLVSTQK

SEQ ID NO: 149 - protein, 9 aa, Homo sapiens
GLVLGPIHK

SEQ ID NO: 150 - protein, 9 aa, Homo sapiens
SPTLGASNR

SEQ ID NO: 151 - protein, 10 aa, Homo sapiens
QALAQISLPR

SEQ ID NO: 152 - protein, 10 aa, Homo sapiens
IANVFTNAFR

SEQ ID NO: 153 - protein, 10 aa, Homo sapiens
QNQIAVDEIR

(19) United States
(12) Patent Application Publication
(10) Pub. No.: US 2014/0220566 A1
(43) Pub. Date: Aug. 7, 2014

(54) MATERIALS AND METHODS FOR DETERMINING SENSITIVITY POTENTIAL OF COMPOUNDS

(71) Applicants: Emma Lahert, Surrey (GB); Stephan Jung, Mainz (DE); Petra Budde, Frankfurt (DE); Hans-Dieter Zucht, Hannover (DE); Stefan Selzer, Losheim am See (DE); Sasa Koncarevic, Sennfeld (DE); Karsten Kuhn, Hofheim am Taunus (DE); Ian Pike, Surrey (GB)

(72) Inventors: Emma Lahert, Surrey (GB); Stephan Jung, Mainz (DE); Petra Budde, Frankfurt (DE); Hans-Dieter Zucht, Hannover (DE); Stefan Selzer, Losheim am See (DE); Sasa Koncarevic, Sennfeld (DE); Karsten Kuhn, Hofheim am Taunus (DE); Ian Pike, Surrey (GB)

(73) Assignee: ELECTROPHORETICS LIMITED, Surrey (GB)

(21) Appl. No.: 14/126,478

(22) PCT Filed: Jun. 18, 2012
(86) PCT No.: PCT/GB2012/051390

§ 371 Date: Mar. 7, 2014

(30) Foreign Application Priority Data
Jun. 17, 2011 (GB) 1110371.0

(51) Int. Cl. G01N 33/50; C12Q 1/68

(52) U.S. Cl. CPC G01N 33/5008; C12Q 1/68; G01N 2500/04; G01N 2500/10
USPC 435/6.11; 435/7.1; 435/7.91

(57) ABSTRACT

The invention concerns in vitro proteomic analysis of cells to determine the sensitizing potential (including allergic potential) of compounds on said cells. Several protein markers are provided that allow assays to be performed to determine whether a chemical has a sensitizing potential of contact and/or respiratory sensitizers.

### FIELD OF THE INVENTION

The present invention relates to in vitro proteomic analysis of cells to determine the sensitizing potential (including allergic potential) of compounds on said cells. Several protein markers have been identified which allow cellular based analysis to determine whether a compound has allergic or irritant potential. Particularly, but not exclusively, the invention provides assays for determining whether a test chemical has sensitizing potential of contact (i.e. on skin) and/or respiratory (i.e. in lung) sensitizers.

### BACKGROUND OF THE INVENTION

Allergy is a type 1 hypersensitive disorder of the immune system. Common allergic reactions include asthma and contact dermatitis. Worldwide the occurrence of allergic diseases is steadily increasing. Allergic disorders have a negative impact on a patient's professional and social life. The costs to the healthcare systems of treating allergic diseases are substantial and increase with the corresponding rise in prevalence. Allergic contact dermatitis (ACD) is accepted to be the most prevalent form of immunotoxicity found in humans. ACD is a T cell mediated delayed skin hypersensitivity which develops after repeated exposure to common metals and a variety of different chemicals and cosmetics. Common chemical contact sensitizers are cinnamaldehyde (CA), dinitrochlorobenzene (DNCB), glyoxal, eugenol, p-phenylenediamine (PPD), and tetramethylthiuram (TMTD). PPD is a chemical substance that is widely used as a permanent hair dye, in textiles, temporary tattoos, photographic developer, printing inks, black rubber, oils, greases and gasoline. Chemical respiratory allergy is less common. However, as respiratory sensitization can lead to asthma it remains a significant challenge. Common chemical respiratory sensitizers are glutaraldehyde, trimellitic anhydride (TMA), diphenylmethane diisocyanate and ammonium hexachloroplatinate.

Many occupational allergens causing allergic contact dermatitis are chemicals (or haptens) that have to bind to a carrier protein to trigger a delayed immune response. Currently, the sensitizing potential of a chemical is assessed in animal experiments such as the guinea pig maximization test (Magnussen and Kligman, 1969) and the local lymph node assay (LLNA) (Kimber et al. 1995). However, the European Directive 86/609/EEC and the 7th Amendment to the Cosmetics Directive enforce an animal testing ban for all cosmetic ingredients since March 2009. Moreover, a marketing ban is in force for cosmetic products containing ingredients tested in animals for all endpoints except repeated dose toxicity, for which the deadline is 2013.

Much research has been devoted to the development of in vitro and in silico predictive testing methods. However, validated in vitro assays for identification and screening of contact sensitizing chemicals are not available.

Chemical allergens are typically small with masses under 1000 daltons, are electrophilic or hydrophilic and can react with nucleophilic amino acids of proteins. Such reactive low molecular weight chemicals can become allergenic when they bind to larger carrier proteins in the body to form hapten-protein conjugates. Some chemical allergens are not inherently allergenic and must undergo metabolic transformation (pro-hapten) or oxidation (pre-hapten) before participating in an allergic response. For example eugenol is considered a pro-hapten, whereas isoeugenol and PPD are classified as pre-haptens.

The skin is the largest organ of the human body and represents a large contact site for potential allergy inducing chemicals. It consists of Langerhans cells (LCs, antigen-presenting dendritic cells), T-lymphocytes, natural killer cells and keratinocytes actively participating in an allergic response. About 95% of all epidermal cells are keratinocytes and are the first cells to encounter foreign antigens. Keratinocytes have an important function in the induction of ACD, as they express metabolizing enzymes. Furthermore, they produce a number of cytokines such as interleukin-18 (IL-18) and tumour necrosis factor alpha (TNF-alpha) inducing migration of LCs to local lymph nodes. Hapten-protein conjugates are recognized by dendritic cells (DCs) which internalize process and transport antigen to the lymph node and present it to T-lymphocytes. After uptake and processing of foreign or self antigens in peripheral tissues, LCs undergo a complex maturation process. Therefore, such test systems comprising keratinocytes and DC models could be useful to develop alternative approaches for predicting the sensitizing potential of chemicals.

The cellular response to irritants and allergens is manifested in two principal ways. Initial exposure is likely to trigger altered gene expression which is subsequently followed by changes in the protein composition of the exposed cells. It is to be appreciated that potential markers of irritant or allergic exposure may be found through the analysis of gene expression or by proteomic analysis of model systems. It is the primary objective of the present invention to provide protein markers whose expression is known to increase or decrease in cells exposed to different classes of chemical compounds. The skilled person would understand that changes in protein levels may also be accompanied, and are often preceded by a parallel change in gene expression and such gene expression changes are within the scope of the present invention.

Whilst there have been very few studies on protein expression changes in model systems for chemical safety testing, there have been several studies on the effects of irritants and allergens on gene expression profiles:

### PBMC-DC

Previous studies have focused on developing in vitro sensitization assays based on analyzing DCs derived from peripheral blood monocytes (PMBC-DC) or from CD34+-stem cells (Tuschl & Kovac, 2001, De Smedt et al. 2002). Initial studies focused on measuring the expression of surface markers following exposure to skin sensitizers (Tuschl et al. 2000). Others have focused on measuring changes in gene transcription during the process of DC maturation.

In a study conducted by Ryan et al. 2004 changes in PBMC-derived DC were analysed after exposure of cells to either 1 mM or 5 mM dinitrobenzenesulfonic acid (DNBS). Comparison of mean signal values from replicate cultures revealed 173 genes that were significantly different (P< or =0.001) between 1 mM DNBS treated and untreated control DC and 1249 significant gene changes between 5 mM DNBS treated and control DC. The expression of up to 60 Genes identified in this screen were further examined by Gildea et al. (2006) by real-time PCR. PBMC-DC were treated with five known skin irritants and 11 contact allergens. This identified 10 genes that were affected by all of the allergens tested such as AK1RC2, ARHGDIB, CCL23, CD1E, CYP27A1, HML2, NOTCH3, S100A4, and signalling lymphocytic activation molecule (SLAM). Other genes (ABCA6, BLNK, CCL4, EPB41L2, TRIM16, and TTRAP) showed an association with the majority of allergens tested.

A targeted microarray comprising 66 immune-relevant genes was developed by Szameit et al. (2008) and tested on PBMC-derived DCs exposed to 2 contact allergens and 1 irritant.

Schoeters et al. 2005 studied the changes in gene expression after exposure of human CD34⁺ progenitor derived DCs to the model allergen dinitrochlorobenzene (DNCB). cDNA microarrays were used to assess the transcriptional activity of 11000 human genes. Compared to control gene expression, changes larger than ±two-fold were observed for 241 genes after exposure to DNCB. Of these genes, 137 were up-regulated and 104 down-regulated. In a subsequent study (Schoeters et al. 2006) gene expression profiles of CD34+-progenitor-derived DCs profiles exposed to nickel sulphate were analyzed. cDNA microarrays were used to assess the transcriptional activity of about 11,000 genes. Significant changes in the expression of 283 genes were observed; 178 genes were up-regulated and 93 down-regulated. In another study Schoeters et al. (2007) analysed changes in gene expression of CD34⁺ progenitor-derived DCs exposed to four contact allergens (nickel sulphate, dinitrochlorobenzene, oxazolone and eugenol) and two irritants (sodium dodecyl sulphate and benzalkonium chloride). A characteristic signature of 25 genes was identified that was specific for the tested allergens, only. From the resulting gene expression data and literature search, 13 genes were selected to develop a multi-marker model to classify and predict the sensitizing potential of chemicals (Hooyberghs et al. 2008). The constructed classifier model is referred to as VITOSENS® and was tested on CD34+-DC.

### DC-Cell Models

Whilst primary LCs isolated from living donor or in vitro differentiated DCs can be used, the widespread use of primary cells in standardized screening assays is limited by donor variations and difficulties to obtain sufficient quantities of cells. Thus, human cell lines with dendritic-like properties are preferably used in the present invention as in vitro differentiation models for predictive skin sensitization tests. Several lymphoid or myeloid cell lines comprising THP-1, UD937 or Mutz-3 cells are currently used as surrogate LC cell lines for skin sensitization testing.

To test whether the VITOSENS® set of 13 gene markers previously identified in DC can be applied to distinguished skin sensitizer and non-sensitizer using the human monocyte-like cell line THP-1, Lambrecht et al. (2009) exposed THP-1 with 5 skin sensitizers and 5 non-sensitizers. However, only a subset of the 13 markers could be confirmed in THP-1 cells, indicating a poor correlation between the results obtained in DC and THP-1 cells. In a similar study Ott et al. (2010) compared the gene expression response of PMBC-derived DC and THP-1 and also found that only 4 (IL-8, TRIM16, CD200R1, GLCM) out of 11 marker (IL-8, CD1e, CD200R1, PLA2G5, TNFRSF11A, AKR1C3, SLC7A11, GCLM, DPYLS3, TFPI, TRIM16) genes found in DC could be confirmed in THP-1 cells.

In another study, Verstraelen et al. (2009) investigated the gene expression response of THP-1 macrophages exposed to 3 respiratory sensitizers, 2 irritants and 1 skin sensitizers. Among the 20 most discriminating genes, EIF4E, PDGFRB, SEMA7A, and ZFP36L2 could be associated with respiratory sensitization.

Another promising alternative to primary DC is the human cell line Mutz-3 which was isolated from a patient with acute myelomonocytic leukemia and shows cytokine dependent proliferation and survival (HU et al. 1996). Phyton et al. (2009) compared the gene expression response of Mutz-3 and PBMC-DC to the sensitizer cinnamaldehyde and found a set of 80 gene markers that overlap between PBMC-DC and Mutz-3. Others evaluated Mutz-3 as a DC model by analysing the cytokine gene expression profile and cell surface expression profile by of DC maturation marker after exposure of Mutz-3 to sensitizer. While the cytokine expression profile correlated with the response of CD34+-DC, the cell surface marker response was less inducible (Nelissen et al. 2009; Williams et al. 2010).

Accordingly, there is still no consensus as to the most appropriate cell lines or genes to use as surrogate screens for chemical safety in vitro.

### SUMMARY OF THE INVENTION

Starting from the assumption that allergic responses will be mediated by changes in protein expression, the present inventors have carried out a detailed proteomic analysis of relevant cell lines such as dendritic cells and keratinocytes exposed to known irritants and sensitizers to reveal putative markers. Surprisingly, there was virtually no overlap between previously reported gene regulations and proteins seen to change in response to exposure with different classes of chemicals. As a result the inventors have defined a small panel of 130 proteins that can serve as objective measures of allergic response in in vitro screens of chemical safety.

The invention allows methods to be carried out for predicting the sensitizing potential of contact and respiratory sensitizers using in vitro methods capable of replacing whole organism testing, based on the measurement of any one or more of these protein markers.

Accordingly, at its most general, the present invention provides materials and methods for determining the sensitising potential of a test compound using in vitro proteomic analysis using one or more of the 130 protein markers identified in Table 1.

The sensitising potential of a compound includes its ability to cause an allergic reaction, e.g. allergenic contact sensitizers or allergenic respiratory sensitizers, or its ability to act as a non-allergenic irritant.

In a first aspect, there is provided an in vitro method for determining the sensitizing potential of a test compound, comprising the steps of

(a) contacting said test compound with a cell;
(b) determining the presence or a change in the level of expression of one or more marker proteins selected from Table 1 in said cell; and
(c) determining the sensitizing potential of said test compound based on said presence or change in level of expression wherein a change in the presence or level of expression of said one or more marker proteins is indicative of said test compound having sensitizing potential.

The presence or a change in level of expression may be determined by establishing the amount of protein marker in the cell or surrounding environment. Alternatively, it may be determined by detecting the presence or amount of nucleic acid sequence encoding said marker protein or form thereof, e.g. mRNA. The presence or increase in either encoding nucleic acid or the protein itself may be measured indirectly. For example, nucleic acid may be extracted from the cell and amplified before quantification. Protein may also be extracted from the cells and enriched and/or labelled prior to quantification.

Table 1 contains 130 protein markers. In preferred embodiments of the invention, the method determines the presence or a change in level of expression of a plurality of these protein markers. Thus, the method according to the first aspect of the invention may determine the presence or change in level of expression of 2, 3, 5, 10, 20, 30, 40, 50, 60, 70, 80, 90, 100, 120 or more protein markers provided in Table 1.

Alternatively, the method may comprises determining the presence or change in level of expression of at least 5%, 10%, 20%, 30%, 40%, 50%, 60%, 70%, 80%, 90% of the protein markers provided in Table 1.

In one embodiment, the method may determine the present or change in level of expression of 100% (i.e. all 130) of the protein markers provided in Table 1.

In a preferred embodiment of the invention and in relation to all aspects described herein, the one or more, or plurality of marker proteins are selected from Table 5. This may be 3 or more, 5 or more, 7 or more, 9 or more, or all 11 marker proteins listed in Table 5.

The method according to this and other aspects of the invention may comprise comparing said presence of level of expression of the one or more protein markers with a reference level. In light of the present disclosure, the skilled person is readily able to determine a suitable reference level, e.g. by deriving a mean and range of values from cells derived from the same, or equivalent cell line. In certain embodiments, the method of this and other aspects of the invention may further comprise determining a reference level for one or more of said marker proteins, above which or below which the presence or amount of said one or more protein markers being expressed in the cell in contact with the test compound can be considered to indicate the sensitizing potential of the compound.

However, the reference level is preferably a pre-determined level, which may for example be provided in the form of an accessible data record.

The test compound may be contacted with any cell. Preferably, the cell is representative of a mammalian skin cell, mammalian lung cell or a cell from a mammalian immune system, e.g. antigen presenting cell such as dendritic cell. More preferably, the cell is obtained from a cell line of mammalian skin cells, e.g. Langerhans cells, keratinocytes; a cell line of lung cells; a cell line of immune system cells such as dendritic cells.

In a preferred embodiment, the cell is from a human cell line with dendritic-like properties. For example, lymphoid or myeloid cell lines such as THP-1, U937 or Mutz-3 cells. THP-1 and U937 can be purchased from the American Type Culture Collection (ATCC, Mansassas, USA) and Mutz-3 cells can be purchased from the Deutsche Sammlung von Mikroorganismen and Zellkulturen GmbH (DSMZ, Braunschweig, Germany).

Many other cells lines will be known to the skilled person.

The method according to this aspect of the invention may be used to determine the contact sensitizing potential of the test compound by determining the presence or a change in expression level of one or more marker proteins provided in Table 1 (A) group 1 when the test compound is contacted with the cell. The one or more marker proteins may be 2, 4, 5, 6, 8, 12 or more selected from Table 1 (A) group 1; or may include all 15 marker proteins.

In a further embodiment, the method may be used to determine the respiratory sensitizing potential of a test compound by determining the presence or a change in expression level of one or more marker proteins provided in Table 1 (B) Group 2 when the test compound is contacted with the cell. The one or more marker proteins may be 2, 4, 5, 6, or 8 or more selected from Table 1 (B) Group 2, or may include all 10 marker proteins.

TABLE 1
Protein markers of chemical sensitizing effect

| | | | | | Change of cell lysate |
|---|---|---|---|---|---|
| SEQ | | IPI Accession | | | protein level on exposure |
| ID No. | Protein Name | Number | Gene name | UniProt ID | to known Sensitizers |
| | A Group 1 | | | | |
| 1 | 6-phosphogluconate dehydrogenase, | IPI00219525 | PGD | 6PGD_HUMAN | Increased |
| | decarboxylating | | | | |
| 2 | Isoform H17 of Myeloperoxidase | IPI00007244 | MPO | PERM_HUMAN | Decreased*⁺ |
| 3 | Isoform 1 of Heat shock cognate 71 kDa protein | IPI00003865 | HSPA8 | Q96BE0_HUMAN | Decreased |
| 4 | Thymosin beta-4-like protein 3 | IPI00180240 | TMSL3 | TMSL3_HUMAN | Decreased* |
| 5 | Voltage-dependent anion-selective channel | IPI00024145 | VDAC2 | VDAC2_HUMAN | Increased |
| | protein 2 | | | | |
| 6 | Protein S100-A8 | IPI00007047 | S100A8 | D3DV37_HUMAN | Decreased*⁺ |
| 7 | Protein S100-A9 | IPI00027462 | S100A9 | D3DV36_HUMAN | Decreased*⁺ |
| 8 | ADP/ATP translocase 2 | IPI00007188 | SLC25A5 | ADT2_HUMAN | Increased |
| 9 | Histone H1.2 | IPI00217465 | HIST1H1C | A8K4I2_HUMAN | Decreased |
| 10 | Peptidyl-prolyl cis-trans isomerase A | IPI00419585 | PPIAL3 | Q567Q0_HUMAN | Decreased |
| 11 | Eosinophil lysophospholipase | IPI00216071 | CLC | C5HZ14_HUMAN | Increased |
| 12 | Transaldolase | IPI00744692 | TALDO1 | TALDO_HUMAN | Increased |
| 13 | Histone H2B type 1-L | IPI00018534 | HIST1H2BL | H2B1L_HUMAN | Decreased |
| 14 | Isoform R-type of Pyruvate kinase isozymes R/L | IPI00027165 | PKLR | KPYR_HUMAN | Increased |
| 15 | Tubulin alpha-4A chain | IPI00007750 | TUBA4A | TBA4A_HUMAN | Decreased |
| | B Group 2 | | | | |
| 16 | Isoform Long of Glucose-6-phosphate 1- | IPI00216008 | G6PD | G6PD_HUMAN | Increased |
| | dehydrogenase | | | | |
| 17 | Isoform B of Serine/threonine-protein kinase 24 | IPI00002212 | STK24 | STK24_HUMAN | Increased |
| 18 | Superoxide dismutase [Cu—Zn] | IPI00218733 | SOD1 | D3DSE4_HUMAN | Decreased*⁺ |
| 19 | ATP synthase subunit a | IPI00654820 | ATPase6/COX3 | Q9B1D3_HUMAN | Increased |
| 20 | 60S ribosomal protein L26 | IPI00027270 | RPL26P33 | Q6IBH6_HUMAN | Increased |
| 21 | Actin-related protein 3 | IPI00028091 | ACTR3 | B4DXW1_HUMAN | Increased |
| 22 | Asparaginyl-tR synthetase, cytoplasmic | IPI00306960 | NARS | SYNC_HUMAN | Increased |
| 23 | Eukaryotic translation initiation factor 3 subunit E | IPI00013068 | EIF3E | EIF3E_HUMAN | Increased |
| 24 | Eukaryotic initiation factor 4A-III | IPI00009328 | EIF4A3 | IF4A3_HUMAN | Increased |
| 25 | Splicing factor, arginine/serine-rich 2 | IPI00005978 | SFRS2 | SRSF2_HUMAN | Decreased |
| | C Group 3 | | | | |
| 26 | Isoform 1 of Splicing factor, arginine/serine-rich 7 | IPI00003377 | SFRS7 | Q564D3_HUMAN | Decreased |
| 27 | Isoform 2 of 4F2 cell-surface antigen heavy chain | IPI00027493 | SLC3A2 | 4F2_HUMAN | Decreased |
| 28 | Alpha-actinin-4 | IPI00013808 | ACTN4 | ACTN4_HUMAN | Decreased |
| 29 | Aminoacyl tR synthetase complex-interacting | IPI00006252 | AIMP1 | AIMP1_HUMAN | Decreased |
| | multifunctional protein 1 | | | | |
| 30 | Protein S100-A4 | IPI00032313 | S100A4 | D3DV46_HUMAN | Decreased |
| 31 | Leukocyte elastase inhibitor | IPI00027444 | SERPINB1 | B4DNT0_HUMAN | Increased |
| 32 | Macrophage-capping protein | IPI00027341 | CAPG | D6W5K8_HUMAN | Decreased |
| 33 | Isoform 2 of Fermitin family homolog 3 | IPI00216699 | FERMT3 | URP2_HUMAN | Increased |
| 34 | Protein disulfide-isomerase | IPI00010796 | P4HB | PDIA1_HUMAN | Decreased |
| 35 | Leucine-rich PPR motif-containing protein, | IPI00783271 | LRPPRC | LPPRC_HUMAN | Increased |
| | mitochondrial | | | | |
| 36 | Isoform 1 of Nuclear autoantigenic sperm protein | IPI00179953 | NASP | NASP_HUMAN | Decreased |
| 37 | Proteasome activator complex subunit 1 | IPI00479722 | PSME1 | Q6FHU3_HUMAN | Decreased |
| 38 | Nucleoside diphosphate kinase | IPI00604590 | NME2 | Q32Q12_HUMAN | Decreased |
| 39 | PRA1 family protein 3 | IPI00007426 | ARL6IP5 | B3KQB4_HUMAN | Increased |
| 40 | Isoform 2 of Spliceosome R helicase BAT1 | IPI00641829 | BAT1 | UAP56_HUMAN | Increased |
| 41 | Isoform Short of DPH:adrenodoxin | IPI00026958 | FDXR | Q6GSK2_HUMAN | Increased |
| | oxidoreductase, mitochondrial | | | | |
| 42 | 26S protease regulatory subunit 6A | IPI00018398 | PSMC3 | PRS6A_HUMAN | Decreased |
| 43 | Stress-70 protein, mitochondrial | IPI00007765 | HSPA9 | GRP75_HUMAN | Decreased |
| 44 | KH-type splicing regulatory protein | IPI00479786 | KHSRP | FUBP2_HUMAN | Decreased |
| 45 | Tu translation elongation factor mitochondrial | IPI00027107 | TUFM | EFTU_HUMAN | Increased |
| | precursor | | | | |
| 46 | R binding protein, autoantigenic (HnRNP- | IPI00011268 | RALY | Q53GL6_HUMAN | Increased |
| | associated with lethal yellow homolog (Mouse)), | | | | |
| | isoform CRA_a (Fragment) | | | | |
| 47 | 40S ribosomal protein S2 | IPI00013485 | RPS2P17 | Q3KQT6_HUMAN | Increased |
| 48 | Isoform Short of TATA-binding protein-associated | IPI00020194 | TAF15 | Q86X94_HUMAN | Decreased |
| | factor 2N | | | | |
| 49 | Thioredoxin domain-containing protein 5 | IPI00171438 | TXNDC5 | Q658S9_HUMAN | Decreased |
| 50 | Cartilage-associated protein | IPI00748502 | CRTAP | B3KME2_HUMAN | Increased |
| 51 | Vesicle-trafficking protein SEC22b | IPI00006865 | SEC22B | SC22B_HUMAN | Increased |
| 52 | Vimentin | IPI00418471 | VIM | D3DRU4_HUMAN | Increased |
| 53 | Isoform 1 of Proteasome subunit alpha type-7 | IPI00024175 | PSMA7 | PSA7_HUMAN | Increased |
| 54 | 40S ribosomal protein S19 | IPI00215780 | RPS19 | B0ZBD0_HUMAN | Decreased |
| 55 | Calreticulin | IPI00020599 | CALR | CALR_HUMAN | Decreased |
| 56 | Phosphatidylethanolamine-binding protein 1 | IPI00219446 | PEBP1 | PEBP1_HUMAN | Decreased |
| 57 | cDNA FLJ60076, highly similar to ELAV-like | IPI00301936 | ELAVL1 | B4DVB8_HUMAN | Decreased |
| | protein 1 | | | | |
| 58 | Transferrin receptor protein 1 | IPI00022462 | TFRC | TFR1_HUMAN | Increased |
| 59 | Bystin | IPI00328987 | BYSL | BYST_HUMAN | Increased |
| 60 | Malate dehydrogenase, mitochondrial | IPI00291006 | MDH2 | Q75MT9_HUMAN | Increased |
| 61 | Non-histone chromosomal protein HMG-17 | IPI00217950 | HMGN2 | HMGN2_HUMAN | Decreased |
| 62 | Cytochrome b-c1 complex subunit 2, | IPI00305383 | UQCRC2 | QCR2_HUMAN | Increased |
| | mitochondrial | | | | |
| 63 | Serine/threonine-protein phosphatase 2A catalytic | IPI00008380 | PPP2CA | B3KUN1_HUMAN | Increased |
| | subunit alpha isoform | | | | |
| 64 | Putative uncharacterized protein | IPI00010402 | SH3BGRL3 | Q86Z22_HUMAN | Decreased |
| 65 | Isoform 2 of Protein disulfide-isomerase A6 | IPI00299571 | PDIA6 | B7Z4M8_HUMAN | Decreased |
| 66 | LM Isoform A of Lamin-A/C | IPI00021405 | LMNA | Q516Y6_HUMAN | Increased |
| 67 | Isoform 1 of DH dehydrogese [ubiquinone] | IPI00028520 | NDUFV1 | Q53G70_HUMAN | Increased |
| | flavoprotein 1, mitochondrial | | | | |
| 68 | 14 kDa protein | IPI00893541 | PDIA3 | | Decreased |
| 69 | Transmembrane protein 33 | IPI00299084 | TMEM33 | TMM33_HUMAN | Increased |
| 70 | Isoform 1 of Neutrophil cytosol factor 4 | IPI00014338 | NCF4 | Q53FG4_HUMAN | Increased |
| 71 | Isoform 1 of Ribonuclease T2 | IPI00414896 | RNASET2 | E1P5C3_HUMAN | Decreased |
| 72 | 60S ribosomal protein L18a | IPI00026202 | RPL18A | RL18A_HUMAN | Increased |
| 73 | Vesicle-associated membrane protein 8 | IPI00030911 | VAMP8 | VAMP8_HUMAN | Decreased |
| 74 | Copine-1 | IPI00018452 | CPNE1 | CPNE1_HUMAN | Increased |
| 75 | Isoform 1 of Protein SET | IPI00072377 | SET | B2RCX0_HUMAN | Decreased |
| 76 | Annexin A5 | IPI00329801 | ANXA5 | D3DNW7_HUMAN | Increased |
| 77 | Elongation factor 1-beta | IPI00178440 | EEF1B2 | EF1B_HUMAN | Decreased |
| 78 | Aspartate aminotransferase, mitochondrial | IPI00018206 | GOT2 | AATM_HUMAN | Increased |
| 79 | ACLY ATP-citrate synthase | IPI00021290 | ACLY | ACLY_HUMAN | Increased |
| 80 | Proteasome 26S non-ATPase subunit 13 isoform | IPI00375380 | PSMD13 | B3KT15_HUMAN | Increased |
| | 2 | | | | |
| 81 | ADP-ribosylation factor 3 | IPI00215917 | ARF3 | ARF3_HUMAN | Increased |
| 82 | Isoform 2 of Filamin-A | IPI00302592 | FLNA | FLNA_HUMAN | Increased |
| 83 | Putative uncharacterized protein RPL7 | IPI00871827 | RPL7P20 | | Increased |
| | (Fragment) | | | | |
| 84 | Alpha-centractin | IPI00029468 | ACTR1A | ACTZ_HUMAN | Increased |
| 85 | 10 kDa heat shock protein, mitochondrial | IPI00220362 | HSPE1 | CH10_HUMAN | Decreased |
| 86 | Putative uncharacterized protein TRAPPC3 | IPI00647089 | TRAPPC3 | | Decreased |
| 87 | 40S ribosomal protein S15a | IPI00221091 | RPS15AP12 | B2R4W8_HUMAN | Increased |
| 88 | Calcium-binding protein 39 | IPI00032561 | CAB39 | A8K8L7_HUMAN | Increased |
| 89 | Trifunctional enzyme subunit alpha, mitochondrial | IPI00031522 | HADHA | Q9UQC5_HUMAN | Increased |
| 90 | Hypoxia up-regulated protein 1 | IPI00000877 | HYOU1 | B7Z766_HUMAN | Decreased |
| 91 | Programmed cell death protein 6 | IPI00025277 | PDCD6 | PDCD6_HUMAN | Increased |
| 92 | GTP-binding nuclear protein Ran | IPI00643041 | RAN | A8K3Z8_HUMAN | Increased |
| 93 | Galectin-1 | IPI00219219 | LGALS1 | Q15954_HUMAN | Increased |
| 94 | Proliferating cell nuclear antigen | IPI00021700 | PCNA | D3DW02_HUMAN | Increased |
| 95 | Histone H1.5 | IPI00217468 | HISTI1H1B | H15_HUMAN | Decreased |
| 96 | Isoform 1 of Triosephosphate isomerase | IPI00465028 | TPI1 | Q53HE2_HUMAN | Decreased |
| 97 | Voltage-dependent anion-selective channel | IPI00216308 | VDAC1P1 | D3DQ93_HUMAN | Decreased |
| | protein 1 | | | | |
| 98 | Thioredoxin | IPI00216298 | TXN | THIO_HUMAN | Decreased |
| 99 | Peroxiredoxin-1 | IPI00000874 | PRDX1 | PRDX1_HUMAN | Increased |
| 100 | Protein S100-A11 | IPI00013895 | S100A11 | S10AB_HUMAN | Increased |
| 101 | Tubulin beta-2C chain | IPI00007752 | TUBB2C | TBB2C_HUMAN | Decreased |
| 102 | Isoform 1 of Heterogeneous nuclear | IPI00216049 | HNRNPK | HNRPK_HUMAN | Decreased |
| | ribonucleoprotein K | | | | |
| 103 | Coronin-1A | IPI00010133 | CORO1A | COR1A_HUMAN | Increased |
| 104 | Heat shock protein HSP 90-beta | IPI00414676 | HSP90AB1 | HS90B_HUMAN | Decreased |
| 105 | 60 kDa heat shock protein, mitochondrial | IPI00784154 | HSPD1 P6 | B9VP19_HUMAN | Increased |
| 106 | F-actin-capping protein subunit alpha-1 | IPI00005969 | CAPZA1 | CAZA1_HUMAN | Increased |
| 107 | HIST2H4A; HIST1H4I; HIST1H4J; HIST1H4E; | IPI00453473 | HIST1H4C | B2R4R0_HUMAN | Decreased |
| | HIST1H4H; HIST1H4D; HIST2H4B; HIST1H4C; | | | | |
| | HIST1H4B; HIST1H4F; HIST1H4L; HIST1H4A; | | | | |
| | HIST1H4K; HIST4H4 Histone H4 | | | | |
| 108 | Hemoglobin subunit epsilon | IPI00217471 | HBE1 | D9YZU7_HUMAN | Increased |
| 109 | ATP synthase lipid-binding protein mitochondrial | IPI00008727 | ATP5G3 | D3DPF0_HUMAN | Increased |
| 110 | Heat shock protein 90 kDa alpha (cytosolic), class | IPI00382470 | HSP90AA2 | HS90A_HUMAN | Increased |
| | A member 1 isoform 1 | | | | |
| 111 | Hemoglobin subunit alpha | IPI00410714 | HBA2 | Q96T46_HUMAN | Increased |
| 112 | Elongation factor 2 | IPI00186290 | EEF2 | EF2_HUMAN | Decreased |
| 113 | HIST2H3D; HIST2H3C; HIST2H3A Histone H3.2 | IPI00171611 | HIST2H3A | H32_HUMAN | Decreased |
| 114 | Transgelin-2 | IPI00550363 | TAGLN2 | TAGL2_HUMAN | Decreased |
| 115 | ATP synthase subunit beta, mitochondrial | IPI00303476 | ATP5B | ATPB_HUMAN | Increased |
| 116 | Fructose-bisphosphate aldolase A | IPI00465439 | ALDOA | ALDOA_HUMAN | Increased |
| 117 | Glyceraldehyde-3-phosphate dehydrogenase | IPI00219018 | GAPDH | G3P_HUMAN | Increased |
| 118 | Actin, aortic smooth muscle | IPI00008603 | ACTA2 | D2JYH4_HUMAN | Decreased |
| 119 | Resistin | IPI00006988 | RETN | D6W649_HUMAN | Increased |
| 120 | Elongation factor 1-alpha | IPI00025447 | EEF1A1 | Q6IQ15_HUMAN | Decreased |
| 121 | Elongation factor 1-alpha 2 | IPI00014424 | EEF1A2 | E1P5J1_HUMAN | Decreased |
| 122 | Eukaryotic translation initiation factor 5A-2 | IPI00006935 | EIF5A2 | IF5A2_HUMAN | Decreased |
| 123 | Hemoglobin subunit zeta | IPI00217473 | HBZ | HBAZ_HUMAN | Decreased |
| 124 | Putative heat shock protein HSP 90-alpha A2 | IPI00031523 | HSP90AA2 | HS902_HUMAN | Increased |
| 125 | cDNA FLJ45706 fis, clone FEBRA2028457, highly | IPI00444262 | NCL | Q6ZS99_HUMAN | Decreased |
| | similar to Nucleolin | | | | |
| 126 | Isoform 2 of Nucleophosmin | IPI00220740 | NPM1 | D3DQL6_HUMAN | Decreased |
| 127 | Peptidylprolyl cis-trans isomerase A-like 4B | IPI00030144 | PPIAL4C | PAL4A_HUMAN | Decreased |
| 128 | Triosephosphate isomerase (Fragment) | IPI00383071 | RCTPI1 | | Increased |
| 129 | 60S ribosomal protein L3-like | IPI00219335 | RPL3L | RL3L_HUMAN | Decreased |
| 130 | TUBA1C protein | IPI00166768 | TUBA1C | Q8N532_HUMAN | Decreased |

(A) Group 1 - top 15 skin sensitizer markers;
(B) Group 2 - top 10 respiratory sensitizer markers;
(C) Group 3 - additional general markers of sensitizing effect
*Change reported is main finding. For some sensitizers an opposite regulation may be seen
⁺A concomitant increase in secreted level of the protein was seen in cell growth medium analysed by ELISA

In accordance with this first and other aspects of the invention, determining the presence or change in expression level of the one or more marker proteins may be achieved in many ways all of which are well within the capabilities of the skilled person.

The determination may involve direct quantification of nucleic acid or protein levels, or it may involve indirect quantification, e.g. using an assay that provides a measure that is correlated with the amount of marker protein present. Accordingly, determining the presence or level of expression of the one or more marker proteins may comprise
(a) contacting the cell with at least one specific binding member that selectively binds to said marker protein or nucleic acid sequence encoding said marker protein; and
(b) detecting and/or quantifying a complex formed by said specific binding member and the marker protein or nucleic acid sequence encoding said marker protein.

The specific binding member may be an antibody or antibody fragment that specifically and selectively binds a marker protein. The determination may include preparing a standard curve using standards of known expression levels of the one or more marker proteins and comparing the reading obtained with the cell contacted with the test compound so as to derive a measure of the change in level of expression of the one or more marker proteins.

A variety of methods may be suitable for determining the presence or changes in level of expression of the one or more marker proteins: by way of a non-limiting example, these include Western blot, ELISA (Enzyme-Linked Immunosorbent Assay), RIA (Radioimmunoassay), Competitive EIA (Competitive Enzyme Immunoassay), DAS-ELISA (Double Antibody Sandwich-ELISA), Liquid Immunoarray technology), immunocytochemical or immunohistochemical techniques, techniques based on the use of protein microarrays that include specific antibodies, “dipstick” assays, affinity chromatography techniques and liquid binding assays. The specific binding member may be an antibody or antibody fragment that selectively binds the protein marker or part thereof. Any suitable antibody format may be employed.

A further class of specific binding members contemplated herein in accordance with any aspect of the invention comprise aptamers (including nucleic acid aptamers and peptide aptamers). Advantageously, an aptamer directed to a protein marker may be provided by a technique known as SELEX (Systematic Evolution of Ligands by Exponential Enrichment), described in U.S. Pat. Nos. 5,475,096 and 5,270,163.

In some embodiments of this and other aspects of the invention, the determination of the presence or the level of expression of one or more of the marker proteins may be performed by mass spectrometry. Techniques suitable for measuring the level of a protein marker selected from Table 1 are readily available to the skilled person and include techniques related to Selected Reaction Monitoring (SRM) and Multiple Reaction Monitoring (MRM) isotope dilution mass spectrometry including SILAC, AQUA (as disclosed in WO 03/016861, the entire content of which is specifically incorporated herein by reference) and TMTcalibrator (as disclosed in WO 2008/110581; the entire content of which is specifically incorporated herein by reference).

WO 2008/110581 discloses a method using isobaric mass tags to label separate aliquots of all proteins in a reference sample which can, after labelling, be mixed in quantitative ratios to deliver a standard calibration curve. A test sample is then labelled with a further independent member of the same set of isobaric mass tags and mixed with the calibration curve. This mixture is the subjected to tandem mass spectrometry and peptides derived from specific proteins can be identified and quantified based on the appearance of unique mass reported ions released from the isobaric mass tags in the MS/MS spectrum.

By way of a reference level, a known or predicted protein marker derived peptide may be created by trypsin, ArgC, AspN or Lys-C digestion of said protein marker. In some cases, when employing mass spectrometry based determination of protein markers, the methods of the invention comprises providing a calibration sample comprising at least two different aliquots comprising the protein marker and/or at least one protein marker derived peptide, each aliquot being of known quantity and wherein said biological sample and each of said aliquots are differentially labelled with one or more isobaric mass labels. Preferably, the isobaric mass labels each comprise a different mass spectrometrically distinct mass marker group.

Accordingly, in a preferred embodiment of the invention, the method comprises determining the presence or expression level of one or more of the marker proteins selected from Table 1 in a cell contacted with a test compound by Selected Reaction Monitoring using one or more determined transitions for known protein marker derived peptides; comparing the peptide levels in the cell under test with peptide levels previously determined to represent contact or respiratory sensitivity by the cell; and determining the sensitivity potential of the test compound based on changes in expression of said one or more marker proteins. The comparison step may include determining the amount of marker protein derived peptides from the treated cell with known amounts of corresponding synthetic peptides. The synthetic peptides are identical in sequence to the peptides obtained from the cell, but may be distinguished by a label such as a tag of a different mass or a heavy isotope.

One or more of these synthetic protein marker derived peptides with or without label for a further aspect of the present invention. These synthetic peptides may be provided in the form of a kit for the purpose of determining the sensitising potential of a test compound.

Other suitable methods for determining levels of protein expression include surface-enhanced laser desorption ionization-time of flight (SELDI-TOF) mass spectrometry; matrix assisted laser desorption ionization-time of flight (MALDI-TOF) mass spectrometry; electrospray ionization (ESI) mass spectrometry; as well as the preferred SRM.

In some embodiments, the determination of the presence or amount of the one or more protein markers comprises measuring the presence or amount of mRNA derived from the cell under test. The presence or level of mRNA encoding the protein marker in the cells contacted with the test compound provides a determination of whether the test compound has a sensitizing potential. Techniques suitable for measuring the level of protein marker encoding mRNA are readily available to the skilled person and include “real-time” reverse transcriptase PCR or Northern blots. The method of measuring the level of a protein marker encoding mRNA may comprise using at least one primer or probe that is directed to the sequence of the protein marker encoding gene or complement thereof. The at least one primer or probe may comprise a nucleotide sequence of at least 10, 15, 20, 25, 30 or 50 contiguous nucleotides that has at least 70%, 80%, 90%, 95%, 98%, 99% or 100% identity to a nucleotide sequence encoding the protein marker provided in Table 1 or Table 5 (and FIG. 10).

Preferably, the at least one probe or primer hybridises under stringent conditions to a protein marker encoding nucleic acid sequence.

The method of the invention may comprises contacting the cell with a binding member as described above, but also includes contacting the binding member with culture medium around the cells which may contain products secreted by the cells. Further, it may be preferably to lyse the cell prior to contact with the binding member to increase contact directly or indirectly with the one or more marker proteins.

The binding members may be immobilised on a solid support. This may be in the form of an antibody array or a nucleic acid microarray. Arrays such as these are well known in the art. The solid support may be contacted with the cell lysate or culture medium surrounding the cell, thereby allowing the binding members to bind to the cell products or secreted products representing the presence or amount of the one or more marker proteins.

In some embodiments, the binding member is an antibody or fragment thereof which is capable of binding to a marker protein or part thereof. In other embodiments, the binding member may be a nucleic acid molecule capable of binding (i.e. complementary to) the sequence of the nucleic acid to be detected.

The method may further comprise contacting the solid support with a developing agent that is capable of binding to the occupied binding sites, unoccupied binding sites or the one or more marker proteins, antibody or nucleic acid.

The developing agent may comprise a label and the method may comprise detecting the label to obtain a value representative of the presence or amount of the one or more marker proteins, antibody or nucleic acid in the cell, cell culture medium or cell lysate.

The label may be, for example, a radioactive label, a fluorophor, a phosphor, a laser dye, a chromogenic dye, a macromolecular colloidal particle, a latex bead which is coloured, magnetic or paramagnetic, an enzyme which catalyses a reaction producing a detectable result or the label is a tag.

The method may comprise determining the presence or level of expression of a plurality of marker proteins or nucleic acids encoding said marker proteins in a single sample. For example, a plurality of binding members selected from Table 1, Table 1 (A) Group 1, Table 1 (B) Group 2, Table 1 (C) Group 3 or a combination thereof or Table 5, may be immobilised at predefined locations on the solid support. The number of binding members selected from Table 1 on the solid support may make up 10%, 20%, 30%, 40%, 50%, 60%, 70%, 80%, 90% or 100% of the total number of binding members on the support.

Alternatively, a plurality of mass features are selected for mass spectrometry techniques described above.

The binding member may be an antibody specific for a marker protein or a part thereof, or it may be a nucleic acid molecule which binds to a nucleic acid molecule representing the presence, increase or decrease of expression of a marker protein, e.g. an mRNA sequence.

The antibodies raised against specific marker proteins may be anti- to any biologically relevant state of the marker protein. Thus, for example, they can be raised against the unglycosylated form of a protein which exists in the body in a glycosylated form, against a precursor form of the protein, or a more mature form of the precursor protein, e.g. minus its signal sequence, or against a peptide carrying a relevant epitope of the marker protein.

In a second aspect of the invention, there is provided a kit for use in determining the sensitizing potential of a test compound in vitro. The kit allows the user to determine the presence or level of expression of an analyte selected from one or more marker proteins or fragments thereof provided in Table 1, one or more antibodies against said marker proteins and a nucleic acid molecule encoding said marker protein or a fragment thereof, in a cell under test; the kit comprising
(a) a solid support having a binding member capable of binding to the analyte immobilised thereon;
(b) a developing agent comprising a label; and, optionally
(c) one or more components selected from the group consisting of washing solutions, diluents and buffers.

The binding member may be as described above. In particular, for detection of a marker protein or fragment thereof, the binding member may be an antibody which is capable of binding to one or more of the marker proteins selected from Table 1, Table 1 (A) Group 1; Table 1 (B) Group 2; or Table 1 (C) Group 3 or a combination thereof.

In a preferred embodiment, the one or more marker proteins are selected from Table 5.

In one embodiment, the kit may provide the analyte in an assay-compatible format. As mentioned above, various assays are known in the art for determining the presence or amount of a protein, antibody or nucleic acid molecule in a sample. Various suitable assays are described below in more detail and each form embodiments of the invention.

The kit may be used in an in vitro method of determining sensitizing potential of a test compound. This method may be performed as part of a general screening of multiple samples, or may be performed on a single sample obtained from the individual.

The kit may additionally provide a standard or reference which provides a quantitative measure by which determination of an expression level of one or more marker proteins can be compared. The standard may indicate the levels of marker protein expression which indicate contact or respiratory sensitivity to said compound.

The kit may also comprise printed instructions for performing the method.

In one embodiment, the kit for the determination of sensitizing potential of a test compound contains a set of one or more antibody preparations capable of binding to one or more of the marker proteins provided in Table 1 or the subset of marker proteins provide in Table 5, a means of incubating said antibodies with a cell exposed to said test compound or extract obtained from said cell, and a means of quantitatively detecting binding of said proteins to said antibodies. The kit may also contain a set of additional reagents and buffers and a printed instruction manual detailing how to perform the method and optionally how to interpret the quantitative results as being indicative of contact or respiratory sensitivity to said compound.

In a further embodiment, the kit may be for performance of a mass spectrometry assay and may comprise a set of reference peptides (e.g. SRM peptides) in an assay compatible format wherein each peptide in the set is uniquely representative of each of the one or more marker proteins described provided in Table 1, Table 1 (A) Group 1; Table 1 (B) Group 2; or Table 1 (c) Group 3 or a combination thereof. Preferably two and more preferably three such unique peptides are used for each protein for which the kit is designed, and wherein each set of unique peptides are provided in known amounts which reflect the levels of such proteins in a standard preparation of said cell exposed to a known sensitizing compound. Optionally the kit may also provide protocols and reagents for the isolation and extraction of proteins from said cell, a purified preparation of a proteolytic enzyme such as trypsin and a detailed protocol of the method including details of the precursor mass and specific transitions to be monitored. Optionally, the kits of the present invention may also comprise appropriate cells, vessels, growth media and buffers.

In a third aspect of the invention, there is provided a method for the diagnosis or prognostic monitoring of contact or respiration sensitizing by an allergen or irritant on an individual exposed to said allergen or irritant the method comprising
(a) determining the presence or level of expression of one or more protein markers selected from Table 1, Table 1 (A) Group 1; Table 1 (B) Group 2; Table 1 (c) Group 3; or Table 5 or a nucleic acid encoding any one or said protein markers or part thereof, in biological sample obtained from said individual.

The biological sample is preferably a sample comprising cells from the individual, e.g. skin cells or lung cells or immune system cells such as dendritic cells. The cells may be lysed and the determination step carried out on the cell lysate. The determination step may be performed as described in the first aspect of the invention.

The method may include determining the presence or level of expression of one or more protein markers in a plurality of biological samples taken over a period of time to create a time line, where contact with the allergen or irritant is time zero.

There is also provided a kit for carrying out the method according to the third aspect of the present invention.

The kit may comprise
(a) a solid support having one or more binding member immobilised thereon, wherein each binding member selectively binds to a protein marker selected from the group provided in Table 1, Table 1 (A) Group 1; Table 1 (B) Group 2; Table 1 (c) Group 3; Table 5 or a nucleic acid encoding the protein marker or fragment thereof;
(b) a developing agent comprising a label; and
(c) one or more components selected from washing solutions, diluents and buffers.

The kit may also comprise printed instructions for performing the method.

The kit may additionally provide a standard or reference which provides a quantitative measure by which determination of an expression level of one or more marker proteins can be compared. The standard may indicate the levels of marker protein expression which indicate contact or respiratory sensitivity to said compound.

Likewise, expression levels of one or more proteins selected from Table 1, Table 1 (A) Group 1; Table 1 (B) Group 2; Table 1 (c) Group 3; or Table 5, may be measured in a tissue sample taken from an individual having been exposed to an allergen or irritant and the levels compared to those from cells having had no exposure to the allergen or irritant; where a change in protein expression level consistent with the changes described in Table 1 is diagnostic of an induced allergy.

The determination of specific proteins whose expression levels are altered following exposure to a chemical sensitizer, e.g. an allergen or irritant, provides for the first time new targets for the diagnosis and treatment of chemically induced allergic conditions such as contact dermatitis and asthma.

Accordingly, in a fourth aspect of the present invention, there is provided the use of one or more protein markers selected from Table 1, Table 1 (A) Group 1; Table 1 (B) Group 2; Table 1 (c) Group 3; or Table 5 for the diagnosis or prognostic monitoring of an individual to chemical sensitizers such as an allergen or irritant.

For example, a plurality of protein markers from Table 1, Table 1 (A) Group 1; Table 1 (B) Group 2; Table 1 (c) Group 3; or Table 5, may be used in a method of monitoring the effectiveness of treatment for skin or respiratory allergy or irritation on a patient suffering from said allergy or irritation. The method may comprise determining changes in the presence or levels of expression of said protein marker (e.g. by a method of the first aspect of the invention), in a tissue sample obtained from said individual prior to treatment and one or more further samples taken post treatment or during the course of treatment; wherein a returning to normal expression levels for the plurality of protein markers is indicative if successful treatment.

In an embodiment of this aspect of the invention, the treatment may be specifically designed to target one or more of the plurality of protein markers selected from Table 1, Table 1 (A) Group 1; Table 1 (B) Group 2; Table 1 (c) Group 3; or Table 5. Accordingly, the invention extends to the provision of the use of one or more protein markers provided in Table 1, Table 1 (A) Group 1; Table 1 (B) Group 2; Table 1 (c) Group 3; Table 5, or parts thereof as targets for treatment for a skin or respiratory allergy.

The invention also includes the use of one or more binding members capable of binding to analytes selected from one or more marker proteins or fragments thereof provided in Table 1, one or more antibodies against said marker proteins and one or more nucleic acid molecules encoding said marker proteins or fragments thereof, for the in vitro diagnosis or prognostic monitoring of an individual to chemical sensitizers.

These binding members are preferably provided on a solid support.

In all aspects of the invention, the methods are in most cases in vitro methods carried out on a sample from a primary cell culture, an established cell line or a biopsy sample taken from a patient suffering from a contact allergy e.g. ACD, or a respiratory allergy or irritation such as asthma. The sample used in the methods described herein may be a whole cell lysate, subcellular fraction e.g. cytoplasm, nucleus, mitochondria, cell membranes, cell culture medium supernatant, tissue or body fluid sample, for example a skin, lung or dendritic cell culture, skin or lung tissue sample, bronchoalveolar lavage (BAL) fluid, blood or a blood product (such as serum or plasma) sample or a urine sample.

Embodiments of the present invention will now be described by way of example and not limitation with reference to the following accompanying figures. All documents mentioned herein are incorporated herein by reference.

### BRIEF DESCRIPTION OF THE FIGURES

FIG. 1: Unsupervised hierarchical clustering using the 83 protein biomarkers (p<=0.05) found in the fourth data set (MeV package).

FIG. 2: PLS loading plot of ANOVA filtered biomarkers found in fourth study.

FIG. 3: PLS score plot for all samples analyzed in the fourth study based in principal components 1 (X-axis) and 2 (y-axis).

FIG. 4: Myeloperoxidase concentration in supernatant of Mutz-3 cells.

FIG. 5: Calprotectin concentration in supernatant of MUTZ-3 cells.

FIG. 6: Zn/Cu-SOD measured in supernatant of Mutz-3 cells.

FIG. 7: PLS-DA model to predict the chemical class membership of an unknown compound. AL: allergen; IR: irritant; Unknown compounds: C, B, E and F.

FIG. 8: Log 2-transformed and referenced fold changes of 83 protein biomarkers. Diamond: allergen; circle: irritant; rectangle: control.

FIG. 9: Differences in the relative abundance of 75 protein biomarkers between TMA and DNCB. Control: rectangle; irritant/SDS: circle; DNCB: triangle; TMA: inverted triangle.

FIG. 10: Table 4—Protein Sequence Table.

FIG. 11: Table 5—SensiDerm Pathway Assay. A suitable skin sensitization assay may comprise a combination of target proteins from Table 1 to approach the involvement of different pathways in the cellular response to chemical allergens.

FIG. 12: Table 6—List of peptides, transition masses and mass spectrometer settings for TSQ Vantage (Thermo Scientific) used in the SRM assay

FIG. 13: Table 7—Statistical testing of SRM data

FIG. 14: ROC curve of SRM data

FIG. 15: PLS-DA model to predict sensitizer samples based on SRM data. U: untreated samples

### DEFINITIONS

The term “antibody” includes polyclonal antiserum, monoclonal antibodies, fragments of antibodies such as single chain and Fab fragments, and genetically engineered antibodies. The antibodies may be chimeric or of a single species.

The term “marker protein” or “biomarker” includes all biologically relevant forms of the protein identified, including post-translational modification. For example, the marker protein can be present in a glycosylated, phosphorylated, multimeric or precursor form.

The term “control” refers to a cultured cell line, primary culture of cells taken from a human or animal subject, or biopsy material taken from a human or animal subject that has been incubated with an equivalent buffer to the test cells but lacking any test compound.

The terminology “increased/decreased concentration . . . compared with a control sample” does not imply that a step of comparing is actually undertaken, since in many cases it will be obvious to the skilled practitioner that the concentration is abnormally high or low. Alternatively, the previously determined normal levels after exposure to non-sensitizing chemicals may be used as a reference value.

The term “antibody array” or “antibody microarray” means an array of unique addressable elements on a continuous solid surface whereby at each unique addressable element an antibody with defined specificity for an antigen is immobilised in a manner allowing its subsequent capture of the target antigen and subsequent detection of the extent of such binding. Each unique addressable element is spaced from all other unique addressable elements on the solid surface so that the binding and detection of specific antigens does not interfere with any adjacent such unique addressable element.

The term “bead suspension array” means an aqueous suspension of one or more identifiably distinct particles whereby each particle contains coding features relating to its size and colour or fluorescent signature and to which all of the beads of a particular combination of such coding features is coated with an antibody with a defined specificity for an antigen in a manner allowing its subsequent capture of the target antigen and subsequent detection of the extent of such binding. Examples of such arrays can be found at www.luminexcorp.com where application of the xMAP® bead suspension array on the Luminex® 100™ System is described.

The term “Compound” means any chemical formulation of elements in any physical state and is to be interpreted in its broadest sense. Within the context of this invention a compound may be a soluble agent such as a pharmaceutical, food additive or cosmetic, gas such as a medical gas, propellant or refrigerant, or solid such as a synthetic or natural polymer, plastic or metal device, medical implant, protective equipment, clothing and may include a mixture of such compounds.

The terms “selected reaction monitoring”, “SRM” and “MRM” means a mass spectrometry assay whereby precursor ions of known mass-to-charge ratio representing known biomarkers are preferentially targeted for analysis by tandem mass spectrometry in an ion trap or triple quadrupole mass spectrometer. During the analysis the parent ion is fragmented and the number of daughter ions of a second predefined mass-to-charge ratio is counted. Typically, an equivalent precursor ion bearing a predefined number of stable isotope substitutions but otherwise chemically identical to the target ion is included in the method to act as a quantitative internal standard. Examples of such methods can be found at http://en.wikipedia.org/wiki/Selected reaction monitoring.

The term “sensitizer” means a chemical that induces an allergic response in exposed people or animals after repeated exposure to the chemical.

“Skin sensitization” means an immunological process which is induced when a susceptible individual is exposed topically to the inducing chemical allergen.

“Sensitizing potential” means the potential of a chemical compound or element to cause skin or respiratory damage through topical exposure which may be by topical exposure or inhalation respectively. For the present purposes, the sensitizing potential of a compound includes its potential to cause damage via an allergic response (a sensitizer) and/or via inflammation (an irritant).

“Irritant” means a chemical that causes an inflammatory effect on living tissue by chemical action at the site of contact. It is important to include irritating chemicals when developing biomarkers for skin sensitization, because sensitizers (i.e. DNCB) can also exert irritation.

Chemicals which do not induce sensitization are referred to as “non-sensitizer”, but may also include irritants.

### DETAILED DESCRIPTION

The need for testing of chemical safety is a long established part of the regulatory process for pharmaceuticals and for the approval for sale of cosmetics and a wide range of other products that come into contact with human skin and mucosa. A number of testing regimes have been established and in some cases only a small number of these tests are proscribed as fit for purpose by national regulators. In the main these tests have been based on whole living organism studies, typically in rodent species.

There is now a strong ethical and economic driver to reduce the number of animals used in pharmaceutical and chemical safety testing and a concomitant need to find suitable in vitro tests to replace the proscribed testing methods. In this context we set out to demonstrate a set of proteins whose levels of expressions within a cultured cell line or tissue biopsy alter in a predictable manner in response to allergenic or irritant compounds.

To discover such a set of proteins we applied a proprietary proteomics discovery workflow to a dendritic cell culture model based on the MUTZ-3 cell line (Masterson et al., Blood. 2002 Jul. 15; 100(2):701-3.) In brief, MUTZ-3 cell lines were cultured in the presence of known allergenic contact sensitizers, allergenic respiratory sensitizers and non-allergenic irritants. In some cases exposure was at a single dose whereas others were exposed to a range of concentrations to look for dose effects. After exposure the MUTZ-3 cells were harvested and lysed and proteins extracted. Following extraction, the total cell lysate was subjected to proteolysis using trypsin and the resultant peptides labelled with one of a sixplex set of isobaric mass tags (Tandem Mass Tags®, Proteome Sciences plc).

Tandem Mass Tags are designed to allow the discriminant labelling of up to six different samples prior to mixing and analysis of all six samples in a single mass spectrometry experiment. Each tag in the set has the same overall mass (isobaric) but on fragmentation in the mass spectrometer releases a unique reporter ion whose intensity relative to the other reporter ions is directly proportional to the relative abundance of the protein in the sample. In our discovery experiments we were able to obtain relative quantitative information for 3173 peptides representing 741 unique proteins consistently measured in at least 50% of all mass spectrometric measurements in a time- and cost-effective manner.

By allowing early mixing of samples the use of Tandem Mass Tags increases the robustness of the data allowing selection of the best candidates for subsequent routine measurement in a targeted screening test with higher throughput than discovery methods. However, to identify those proteins whose expression is predictably altered by chemical exposure it is necessary to undertake a panel of statistical analyses such as supervised and un-supervised cluster analysis. Through selective application of a range of such statistical tools we identified 130 protein markers that were significantly regulated in response to exposure to a set of training chemicals. Within these 130 proteins are markers of contact and respiratory sensitizers/allergens as well as non-sensitizing irritants. Following the identification of candidate biomarkers using a set of training chemicals we developed a classification model that could predict whether a compound was a sensitizer/allergen or a non-sensitizing irritant based on the detected amount of one or more of the 130 biomarkers listed in Table 1. This classification model can be used to interpret the protein expression data from MUTZ-3 cells exposed to unknown chemicals or combinations of chemicals and to assign said chemicals into the allergen or irritant group. This test system is therefore suitable to replace living, whole-organism test for chemical safety.

It is recognised that the discovery methods used in this study are less well suited to the routine analysis of hundreds, thousands or tens of thousands of chemicals with unknown safety profile, such as will be needed to meet the ethical need to replace animal testing and the pending EU legislation. To overcome this potential bottleneck it will be necessary to provide more targeted means of analysis of one or more of the 130 protein biomarkers listed in Table 1. There are a number of suitable methods for the targeted measurement of up to 130 different proteins in a single analysis. One such technology is immunoassay where antibodies with specificity for the biomarker protein are used to capture, detect or capture and detect the protein. Immunoassay formats include but are not limited to enzyme-linked immunosorbent assay (ELISA), antibody sandwich ELISA, competitive ELISA, immunoPCR and Western blot. Where a small number of proteins are to be measured it is possible to use individual tests such as ELISA or western blot for each protein. Alternatively, multiplex testing methods such as antibody arrays and/or bead suspension arrays where a plurality of biomarkers are detected and quantified simultaneously can be used.

In some cases it may be undesirable or impossible to use antibodies to selectively quantitate levels of protein expression. Examples include where the biomarker is post-translationally modified as a result of chemical exposure and such modification is immunologically inert, or where proteolytic activity causes degradation of a protein thereby destroying epitopes recognised by available antibodies. In such situations non-antibody binding agents such as aptamers may be used. More preferably, quantitative mass spectrometry methods can be developed based on the principle of selected reaction monitoring (SRM).

In an SRM method peptides representing the target marker protein are selected based on empirical data obtained during marker discovery or are designed using in silico tools. Typically a combination of the two approaches is used for best results. For absolute quantitation by SRM it is necessary to provide an external equivalent ‘heavy’ peptide that is isotopically distinct to the native form to be measured in the analytical sample. There are a number of different approaches for the provision of such isotopically distinct reference peptides though they all share the common feature of adding one or more heavy stable isotopes into the peptide during production. The simplest approach which is often termed ‘AQUA’ is to use an amino acid containing one or more stable isotopes of hydrogen, carbon, nitrogen or oxygen. Typically a combination of isotopes is used to introduce a total mass difference of between 6 and 10 Daltons per peptide. There are a number of commercial sources of such heavy AQUA peptides (e.g. Thermo Scientific (www.thermoscientific.com)). An alternate to AQUA is to add heavy isotopes through a covalent label attached to a standard synthetic peptide. Such methods have the advantage of speed and cost of production of the reference peptides. However, the method then requires use of an isotopically distinct but chemically identical tag to label each analytical sample. There are a number of approaches for tag-based SRM methods including mTRAQ® (ABSciex) and TMT® (Thermo Scientific). Where multiple reference peptides are required it is possible to manufacture a synthetic gene encoding all desired peptides in a concatamer polypeptide. This is then transfected into a suitable expression host or in vitro transcription system and the expressed polypeptide purified prior to cleavage to release the individual reference peptides. Typically this would be performed using a heavy amino acid to provide the isotope substitution such that all peptides are ‘heavy’ relative to the natural form in the analytical sample. An example of such a method is the QCONCAT system (Pratt et al. Nature Protocols 1,-1029-1043 (2006)).

It will be understood by the skilled practitioner that the method of detection is not particularly limiting to the present invention and all methods of relative or absolute quantitation of the target proteins are incorporated herein.

The human MUTZ-3 cell lines were used as a DC cell culture model for developing a protein biomarker based in vitro assay system for determining the sensitizing potential of chemical sensitizers. It is consequently an additional aspect of the invention that these protein markers can also be used for the diagnosis of respiratory or skin allergy in a mammal or human suspected of suffering from such an allergy. In this context a suitable tissue sample such as biopsy samples of skin or bronchoalveolar lavage are collected, proteins extracted and measured according to one of the methods of the present invention. The levels detected in the said sample are then compared with the levels known to be associated with a response to sensitizing agents in the MUTZ-3 cell line.

It is a further aspect of the invention that the presently disclosed proteins provide alternate means for the treatment of chemically induced allergy such as contact dermatitis and asthma.

The invention is further illustrated by the following experiments.

### Example 1

### Proteomic Analysis of a Dendritic Cell Model

### Training Chemicals Used for the Discovery of Candidate Biomarkers

A set of training chemicals was selected for biomarker discovery in human Mutz-3 cells. The selected chemicals comprised 5 skin sensitisers of different strength, 2 respiratory sensitizer and 3 non-sensitisers/irritants (Table 2).

### Contact Sensitizers/Allergens:

DNCB: 1-chloro-2,4-dinitrobenzene (DNCB), is a organic compound used in color photography processing. DNCB is considered an extreme allergen.

Oxazolone: 4-ethoxymethylene-2-phenyloxazol-5-one is considered an extreme chemical allergen.

PPD: Para-phenylenediamine is a strong chemical allergen. PPD is widely used as a permanent hair dye, in textiles, temporary tattoos, photographic developer, printing inks, black rubber, oils, greases and gasoline. PPD oxidation is a precondition for DC activation.

Eugenol: Eugenol is a member of the phenylpropanoids class of chemical compounds. It is a clear to pale yellow oily liquid extracted from certain essential oils especially from cinnamon and basil. Eugenol is used in perfumeries, flavorings, essential oils and in medicine as a local antiseptic and anesthetic. Eugenol is considered a pro-hapten which must be metabolized before it can elicit an allergic response.

Cinnamic aldehyde: 3-phenyl-2-Propenal; Cinnamal, Cinnamaldehyde is an oily yellow liquid with strong odor of cinnamon. This compound is the main component of cinnamon oil. The predominant application for cinnamaldehyde is in the flavor and fragrance industries. It is used as a flavouring for chewing gum, ice cream, candy, and beverages. Cinnamic aldehyde is considered a moderate sensitizer.

### Respiratory Sensitizers/Allergens:

TMA: Trimellitic anhydride is a very reactive chemical which is industrially used to synthesis trimellitate esters. These esters are used as plasticizers for polyvinyl chloride, especially when temperature stability is required, e.g., in wire and cable coatings. It is considered a respiratory sensitizer.

MDI: 2, 4-Diphenylmethane diisocyanate is a reactive material used to reduce polyurethane compounds. BASF's trademarks for MDI are Lupranate® (North America) and Lupranat® (Europe). MDI is a respiratory sensitizer.

### Irritating/Non-Sensitizing Chemicals:

SDS: Sodium sodium dodecyl sulfate (SDS), lauryl sulfate (SLS) or sodium laurilsulfate is an anionic surfactant used in many cleaning and hygiene products. SDs can cause skin and eye irritation.

Phenol: also known as carbolic acid is an organic compound. The major uses of phenol involve its conversion to plastics or related materials. Phenol is corrosive to the eyes and the skin.

Salicylic acid: is also known as 2-hydroxybenzenecarboxcylic acid. Salicylic acid is known for its ability to ease aches and pains and reduce fevers. Salicylic acid is a key ingredient in many skin-care products for the treatment of acne, psoriasis, calluses, corns, keratosis pilaris, and warts. Because of its effect on skin cells, salicylic acid is used in several shampoos used to treat dandruff. Exposure to salicylic acid can cause hypersensitivity.

### Cell Culture

The human myeloid leukaemia-derived cell line MUTZ-3 (DSMZ, Braunschweig, Germany, Hu et al. 1996) requires addition of cytokines and growth factors to the culture medium for proliferation and survival. MUTZ-3 progenitor cells were cultured in α-MEM containing L-glutamine and nucleotides (Invitrogen, 22571-020) supplemented with 20% FCS and 40 ng/ml GM-CSF. The cells are grown at 37° C. and 5% CO₂ and the media is changed three times a week. The cells are kept at a concentration of 200000 cells/ml. When splitting the cells they are centrifuged at 800 rpm for 5 minutes. The supernatant is removed and the cells are carefully re-suspended in 1 ml of medium and counted to set the proper concentration.

### Chemical Exposure of Cells

TABLE 2
List of reference chemicals used to discover
biomarkers

| | Chemical/ | Final test |
|---|---|---|
| Chemical | Solvent | concentration |
| Dimethylsulfoxide | DMS0 | 0.1% (v/v) |
| Diphenyl-methane-4,4′- | MDI [μM] in | 150 μM |
| diisocyanate (MDI) | DMSO | |
| Trimellitic anhydride (TMA) | TMA [μM] in | 500 μM |
| | DMSO | |
| Dinitrochlorobenzene (DNCB) | DNCB [μM] in | 4 μM, 8 μM, 10 μM |
| | DMSO | |
| Cinnamic aldehyde (CA) | CA [μM] in | 50, μM, 100 μM, 120 |
| | water | μM, 200 μM |
| Para-phenylenediamine (PPD) | PPD [μM] in | 75 μM, 150 μM |
| | DMSO | |
| Eugenol | Eugenol [μM] | 500 μM |
| | in DMSO | |
| Oxazolone | Oxa [μM] in | 250 μM |
| | DMSO | |
| Sodium dodecyl sulphate | SDS [μM] in | 200 μM, 300 μM, 600 |
| (SDS) | water | μM |
| Salicylic acid (SA) | SA [μM] in | 500 μM |
| | DMSO | |
| Phenol | Phenol [μM] | 500 μM |
| | in water | |

MUTZ-3 cells were grown in cell culture medium at a cell density of 2×10⁵ cells/ml. Test chemicals were added and the plates were incubated for 24 hours at 37° C. in a 5% CO2 humidified incubator. When a chemical was dissolved in DMSO, a final concentration of 0.1% DMSO was used in the relevant negative control. After 24 h incubation, cells were harvested and washed twice in PBS.

The cells are harvested and centrifuged to obtain serum free conditions during two hours prior to exposure. The chemicals are added at various concentrations to MUTZ-3 progenitor or iMUTZ3 DC (Table 2). A 1000× stock solution is freshly prepared on the day of exposure. Chemicals unable to dissolve in water are dissolved in DMSO with a maximum final in-well concentration of 0.1%. Additionally to the test chemicals, medium and vehicle controls were also prepared. After addition of the appropriate test or control medium, the cells were incubated at 37° C. in a closed incubator with an atmosphere containing 5% CO₂ for 48 hours. After exposure, spent medium was removed and stored for targeted measurement of inflammatory markers whilst the cells were prepared for proteomic biomarker discovery.

### Cell Lysis and Sample Preparation

Cells were lysed in four volumes of 100 mM TEAB (triethylammonium bicarbonate), pH 8.5+0.1, 1 mM TCEP (tris[2-carboxyethyl]phosphine*HCl), 0.1% SDS. After suspending the cell pellet in lysis buffer the suspension was heated at 95° C. for ten minutes in a thermomixer ([Eppendorf, Thermomixer comfort). Cell lysates were sonicated twice on ice for two minutes followed by a second cycle heating and sonication. Samples were then centrifuged at 14.000 g for ten minutes and supernatants were used for further analyses or stored at −80° C.

### Determination of Protein Concentrations

The protein concentration was determined using the Bradford reagent. The results were calculated using a standard curve created from measurements of dilutions of a standard consisting of BSA/IgG (50%/50%).

### Trypsin Digestion and Isobaric Mass Tag Labelling

Tandem Mass Tags (TMTs) (Thermo Scientific) comprise a set of amine-reactive isobaric labels, which are synthesized with heavy and light isotopes to present the same total mass but to provide reporter-ions at different masses after activation with collision-induced dissociation (CID) and subsequent tandem mass spectrometry (MS/MS).

Equivalents of up to 100 μg protein solution per sample were used for proteomics profiling experiments. A reference pool was created from an aliquot of all samples and included in each TMTsixplex labelling reaction. The final volume of the sample was adjusted with TMT labelling buffer (100 mM TEAB pH 8.4-8.6, 0.1% SDS) to 100 μl per sample. The samples were reduced for 30 min at room temperature by the addition of 5.3 μL each of 20 mM TCEP in water and subsequently alkylated for 1 h at room temperature by the addition of 5.5 μL each of 150 mM iodoacetamide in acetonitrile.

For protein digestion, 10 μL of a 0.4 μg/μL trypsin solution (sequencing grade modified trypsin, Promega) in 100 mM TEAB buffer pH 8.4-8.6 was added to each vial and incubated at 37° C. for 18 h. The digested protein samples were labelled with the TMTsixplex reagents TMT6-126, TMT6-127, TMT6-128, TMT6-129, TMT6-130 and TMT6-131). TMTsixplex reagents were dissolved in acetonitrile to yield a concentration of 60 mM and 40.3 μL of the corresponding reagent solution were added to the sample vials and samples incubated for 1h at room temperature. To reverse occasional labelling of Tyr, Ser and Thr residues, 8 μL of an aqueous hydroxylamine solution (5% w/v) was added and incubated for 15 min at room temperature. The TMTsixplex-labelled samples were combined and purified.

### Sample Purification

The samples were diluted with 3 mL water/acetonitrile 95:5+0.1% TFA each and desalted using HLB Oasis cartridges (1 cc, 30 mg, Waters). The eluate fraction each was further purified by strong cation exchange using self-made cartridges (CHROMABOND empty columns 15 ml, Macherey-Nagel, filled with 650 μL SP Sepharose Fast Flow, Sigma). After loading the peptides and washing with 4 mL water/acetonitrile 75:25+0.1% TFA, the peptides were eluted with 2 mL H₂O:ACN 75:25+400 mM ammonium acetate. The samples were dried in a vacuum concentrator and dissolved in 50 μL water/acetonitrile 95:5+0.1% TFA each and stored at −20° C. until analysis.

### Liquid Chromatography and Tandem Mass Spectrometry (LC-MS/MS)

The TMTsixplex labelled samples were measured by High-Performance Liquid Chromatography-Tandem Mass Spectrometry (HPLC-MS/MS). For example, 5 μL (5 μg) of each sample were injected and measured using an electrospray ionization linear ion trap quadrupole Orbitrap mass spectrometer (Thermo Scientific) coupled to a Proxeon EASY-nLC (Thermo Scientific). After loading and washing of the sample during 10 min with H2O:formic acid (99.9%/0.1%) on a self-packed 0.1×20 mm trap column packed with ReproSil C18 (5 μm particles, Dr. Maisch), the separation was run for 90 min using a gradient of H2O:formic acid (99.9%/0.1%; solvent A) and acetonitrile:formic acid (99.9%/0.1%; solvent B) at a flow rate of 300 nL/min. A 0.075×150 mm column was self-packed with ReproSil C18 (3 μm particles, Dr. Maisch). All mass spectra were acquired in positive ionization mode with an m/z scan range of 350-1800 using a top ten HCD method for fragmentation.

### Peptide and Protein Identification and Quantification

Peaks lists were generated from Orbitrap raw data files as mascot generic files (*.MGf data files) using Proteome Discoverer (version1.1; ThermoFisher, San Jose, USA). The resulting *.mgf files were searched against the IPI human database (version 3.68 from February 2011) by MASCOT (version 2.2; MatrixScience, London, UK (Probability-based protein identification by searching sequence databases using mass spectrometry data. Perkins D N, Pappin D J, Creasy D M, Cottrell J S. Electrophoresis. 1999 December; 20(18):3551-67.)). Peptide and protein identification was performed using the following parameters:

Carbamidomethyl at Cysteines and TMT modifications at N-terminal site and at Lysines were set as fixed modifications. Trypsin was used for the enzyme restriction, with three allowed miscleavages and an allowed mass tolerance +/−10 ppm for the precursor masses and 0.05 for the fragment ion mass. The corresponding MASCOT result files (*.dat data file) were downloaded and reporter ion intensities and protein identifications were extracted with an in-house tool. Reporter ion intensities and protein identities were exported into a relational MySQL database (version 5.157; Oracle, Redwood Shores, USA) and log 2 ratios of reporter ions were calculated.

### Data Pre-Processing of Extracted Reporter Ion Intensities and Relative Quantitation

The six reporter ion intensities of the isobaric mass tags were corrected for isotopic distribution and systematic bias by means of sum scaling based on the assumption of a constant integral of any reporter ion series within one LC/MS/MS run. In addition, those MS/MS scans were filtered out where the reporter ion intensity of all six tags was smaller than 80 AU (arbitrary units) and where the reporter ion intensity of less than two tags was smaller than 10 AU. The relative intensities of reporter ions represent the relative amount of a peptide in the sample. To compare the relative amount of a peptide to all samples, a ratio is calculated between each sample versus the pooled reference sample. The ratio was log 2 transformed to yield referenced measurement values for each peptide. To obtain information on relative changes on the protein level, the log 2 reference reporter ion intensities for each identified peptide belonging to one protein identity were averaged as the geometric mean.

### Example 2

### Statistical Analysis and Generation of a Classification Model

Reference chemicals belonging to the groups of sensitizer and irritant as well as appropriate (vehicle) controls were used to select candidate protein biomarkers. These chemicals were applied in different combinations and concentrations in four analytical discovery studies.

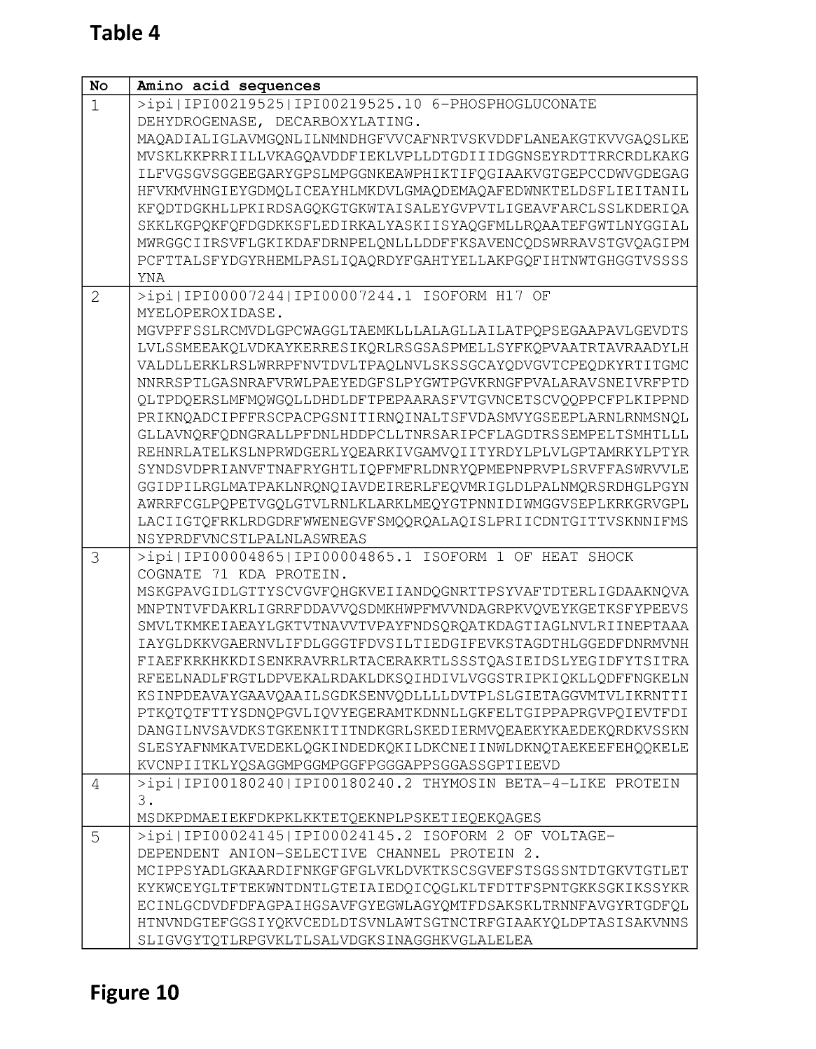

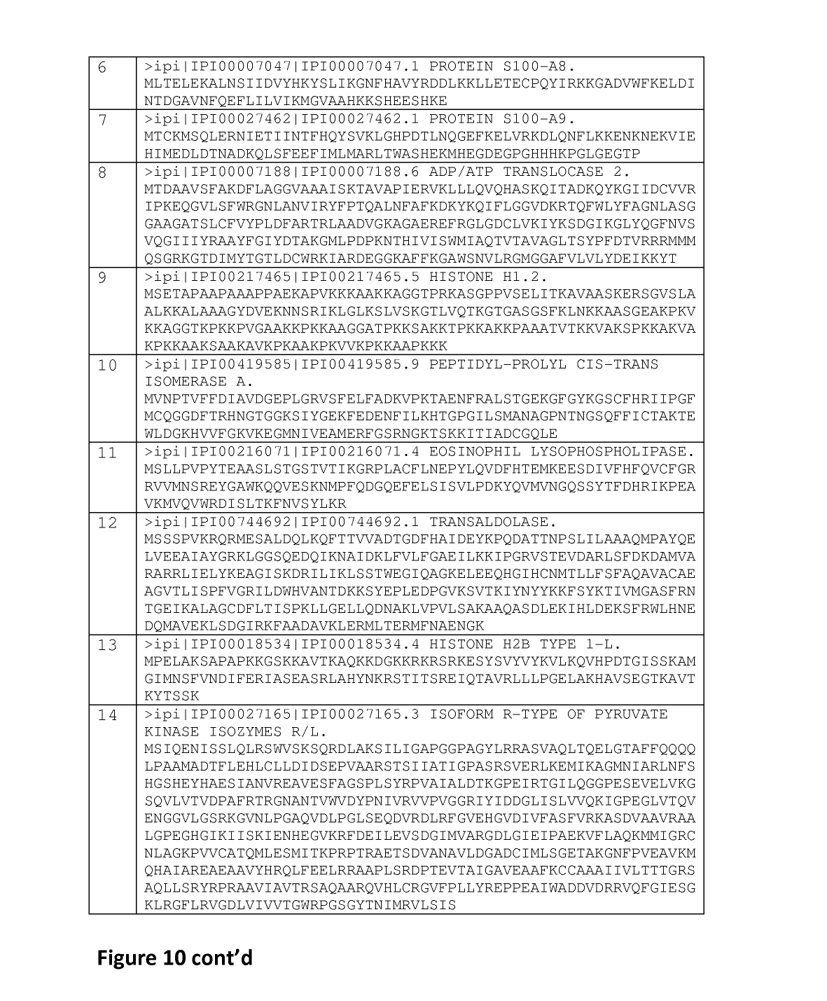

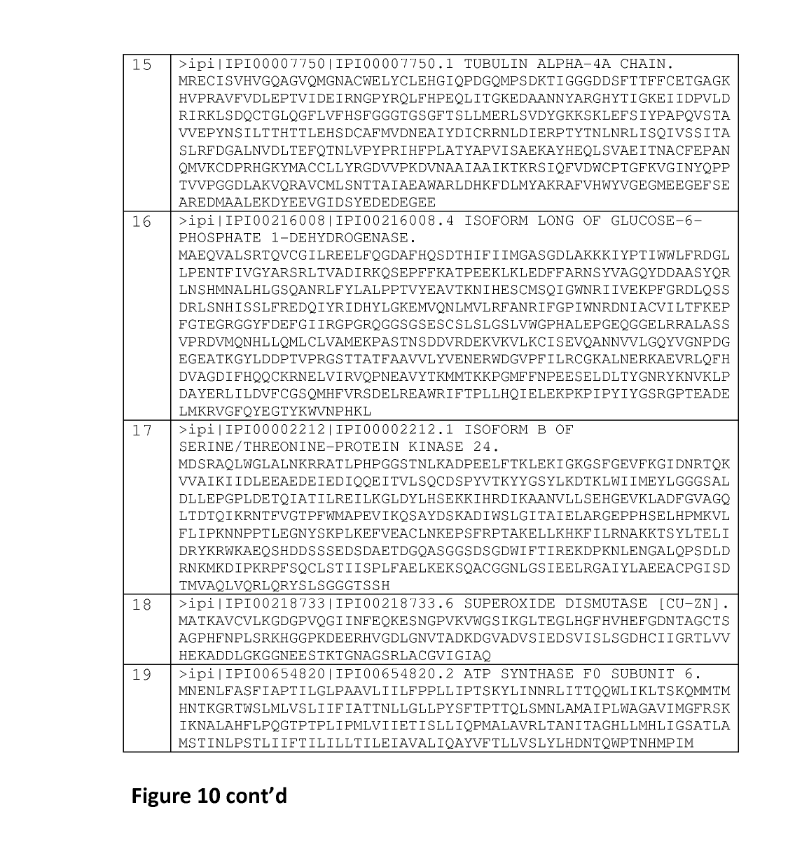

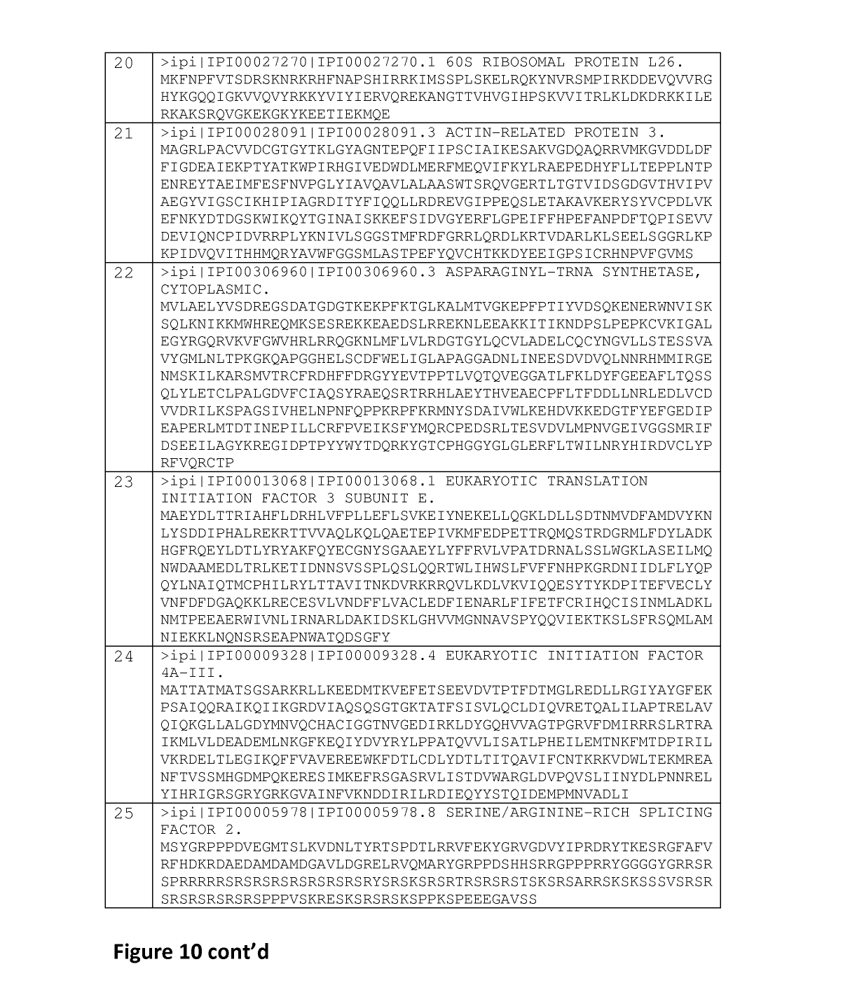

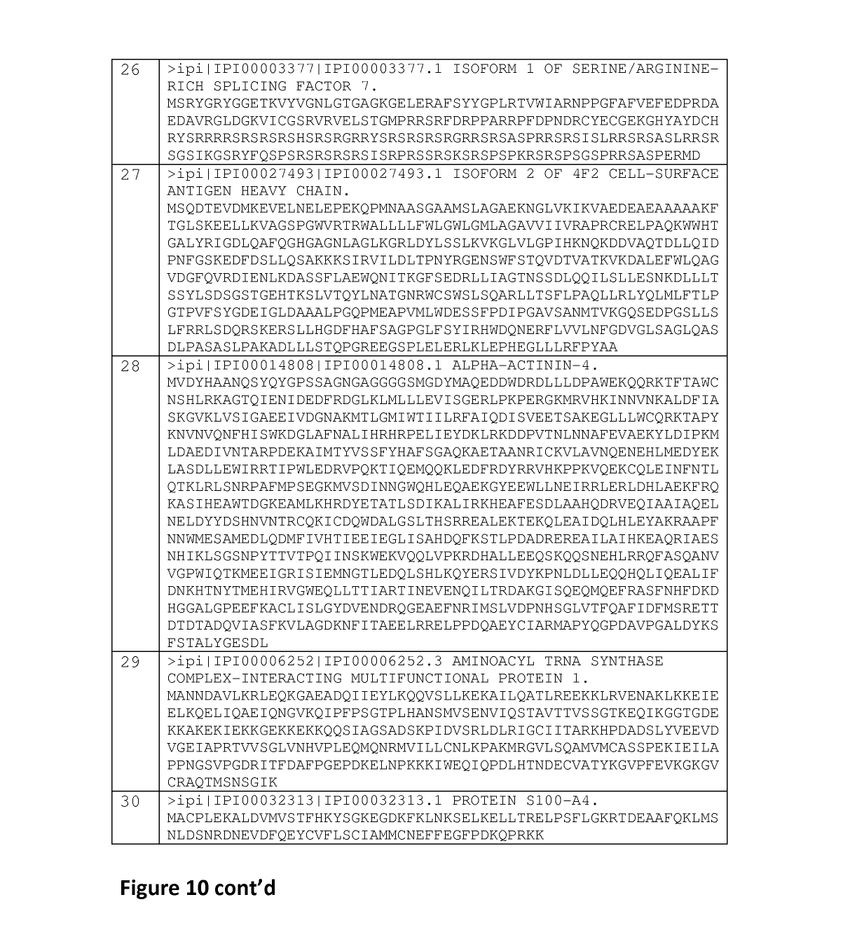

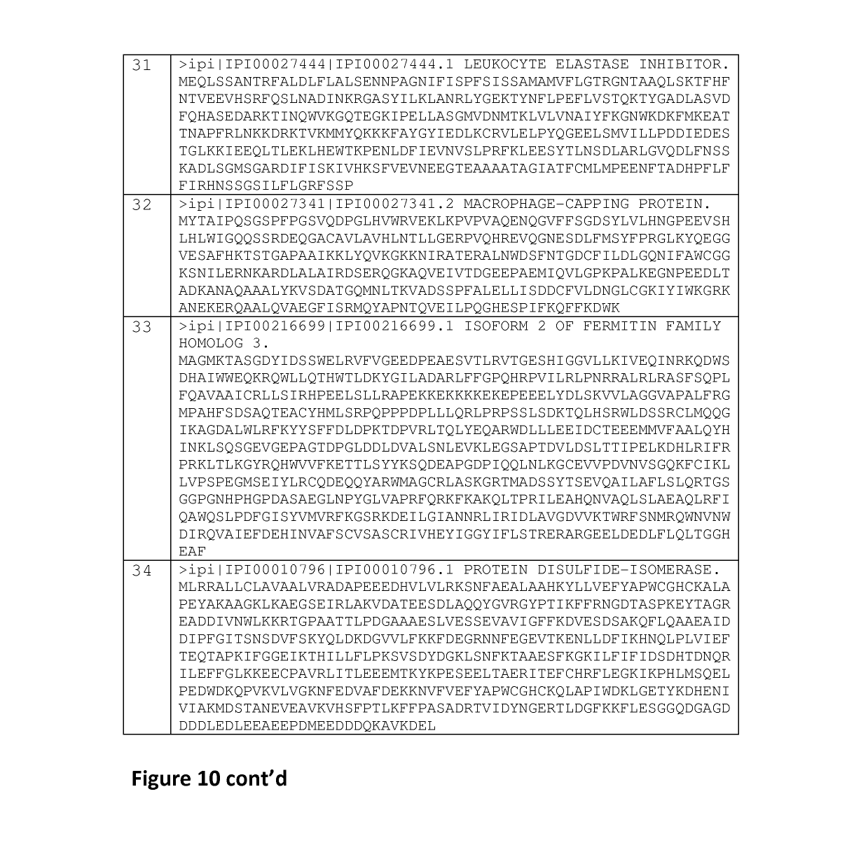

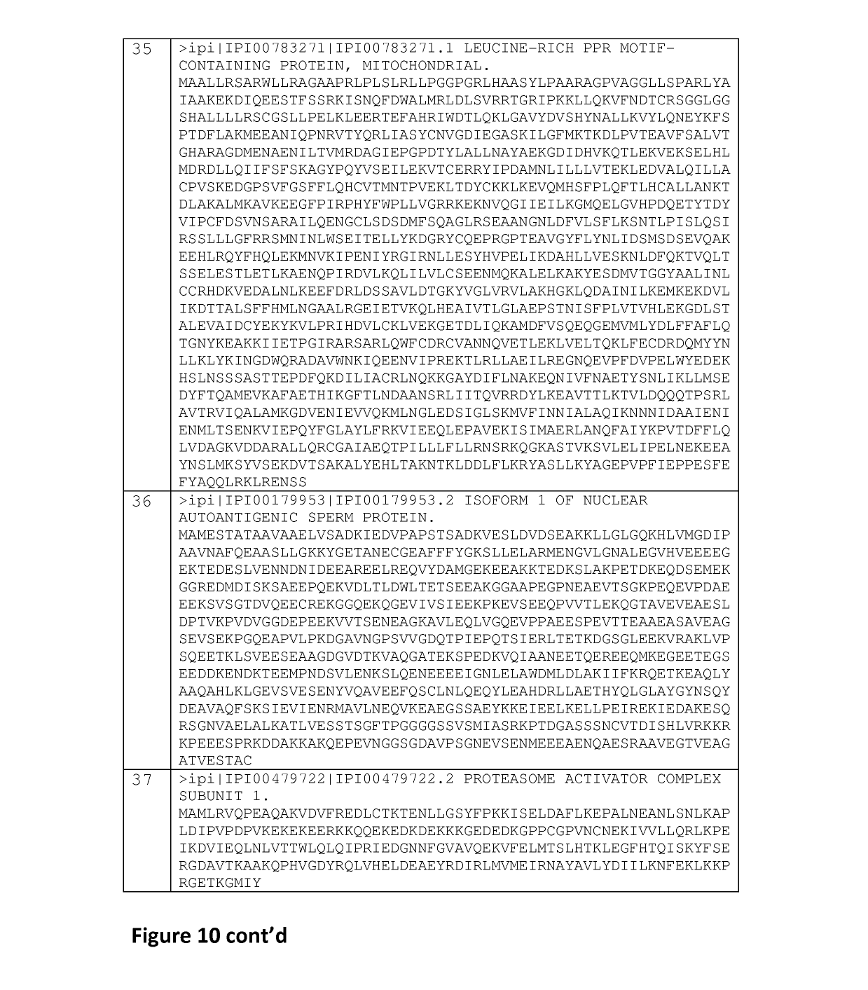

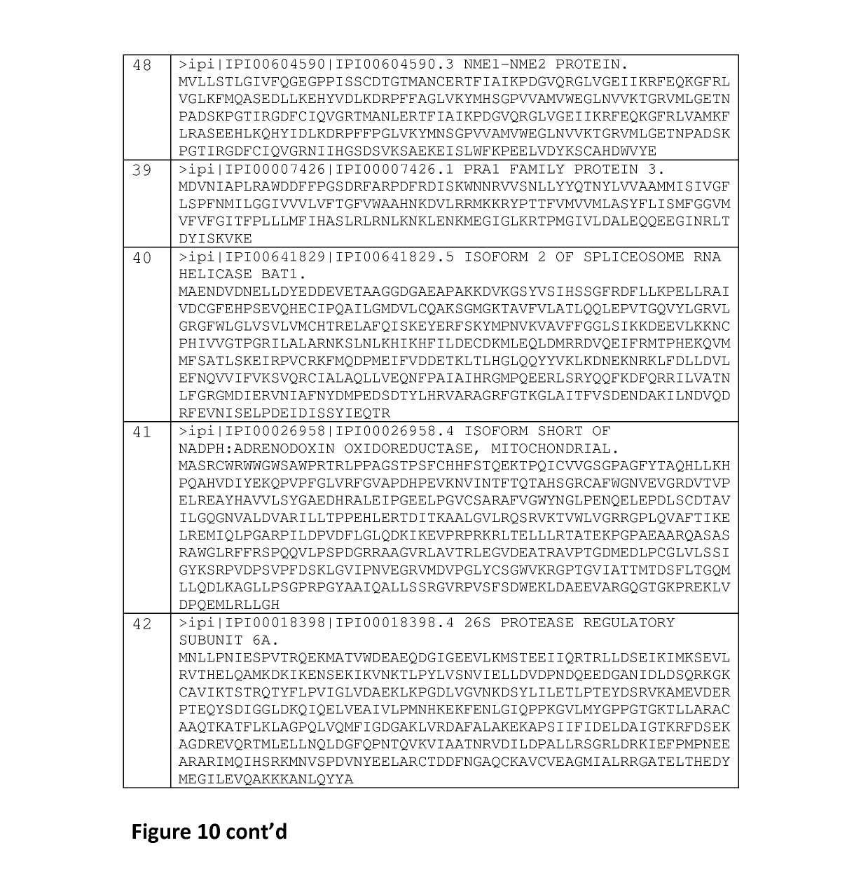

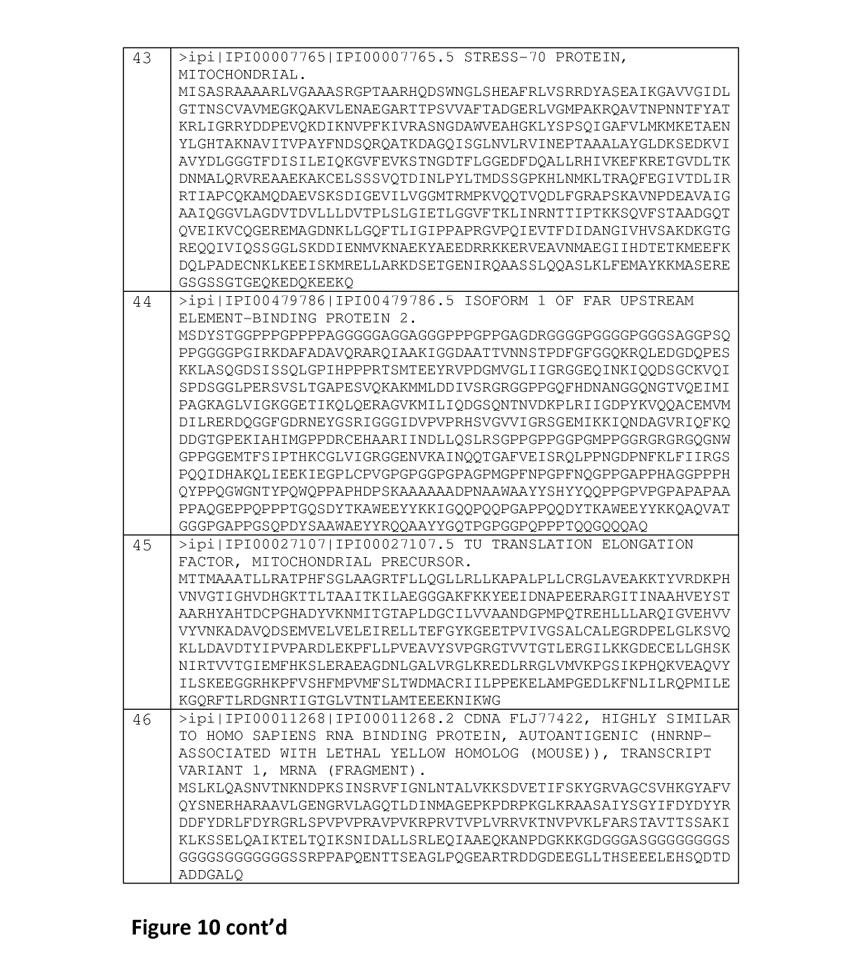

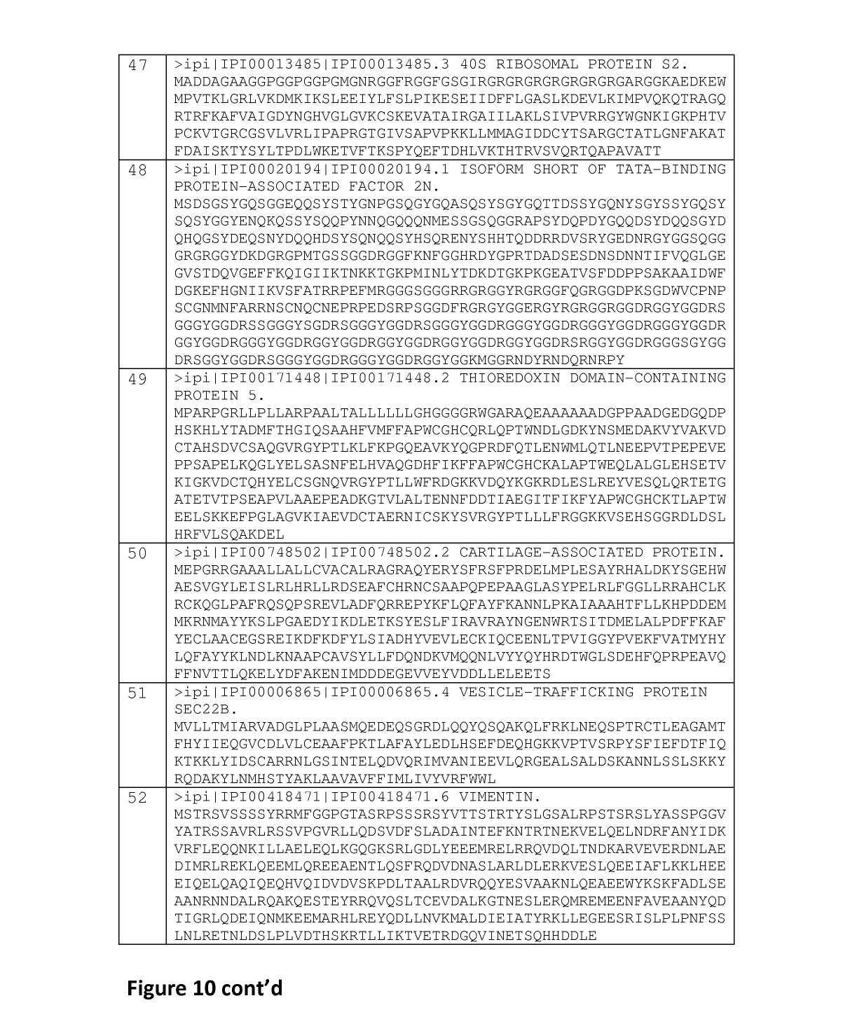

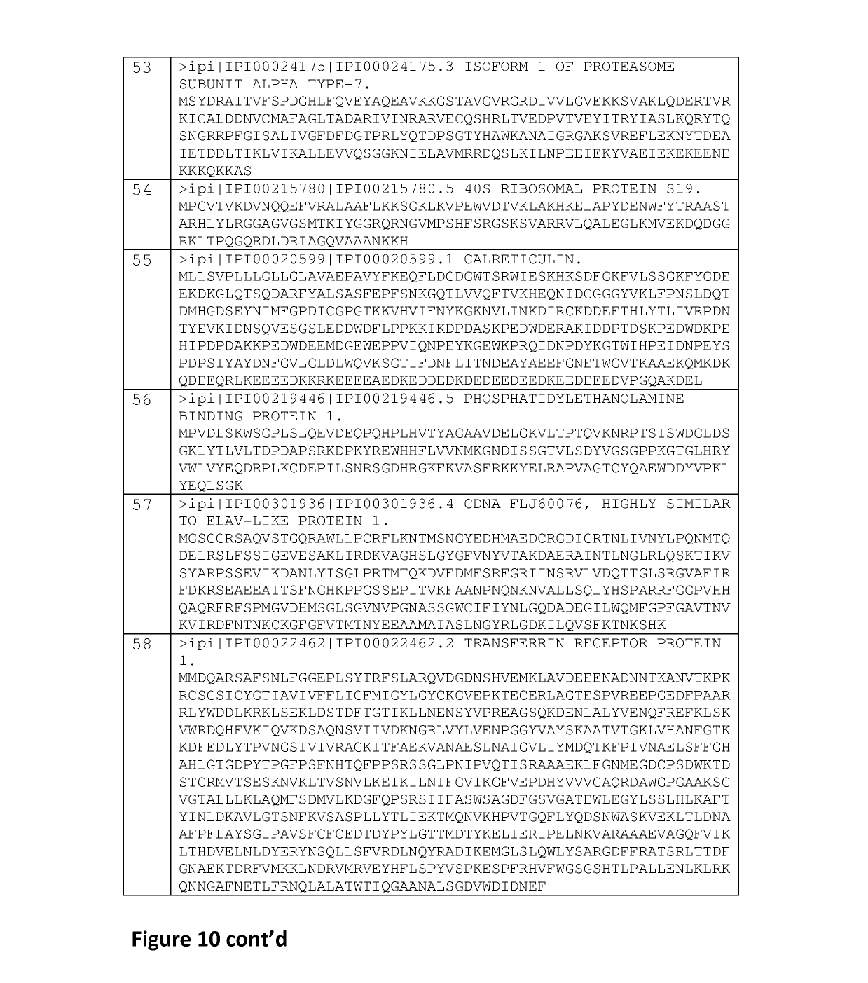

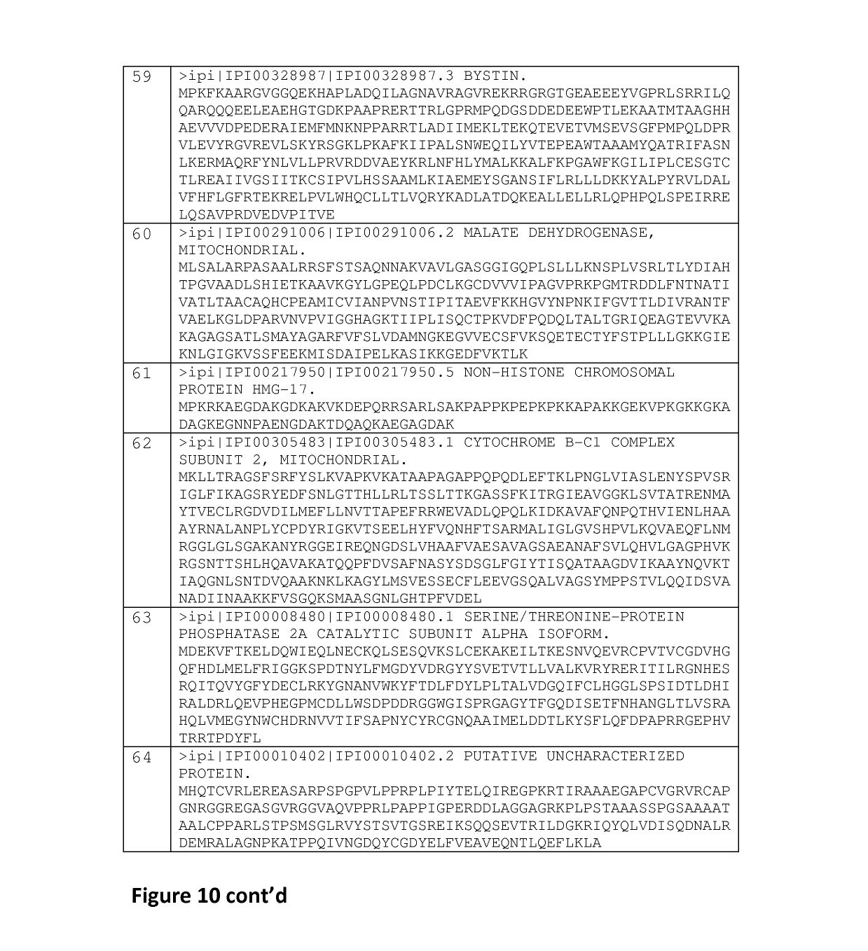

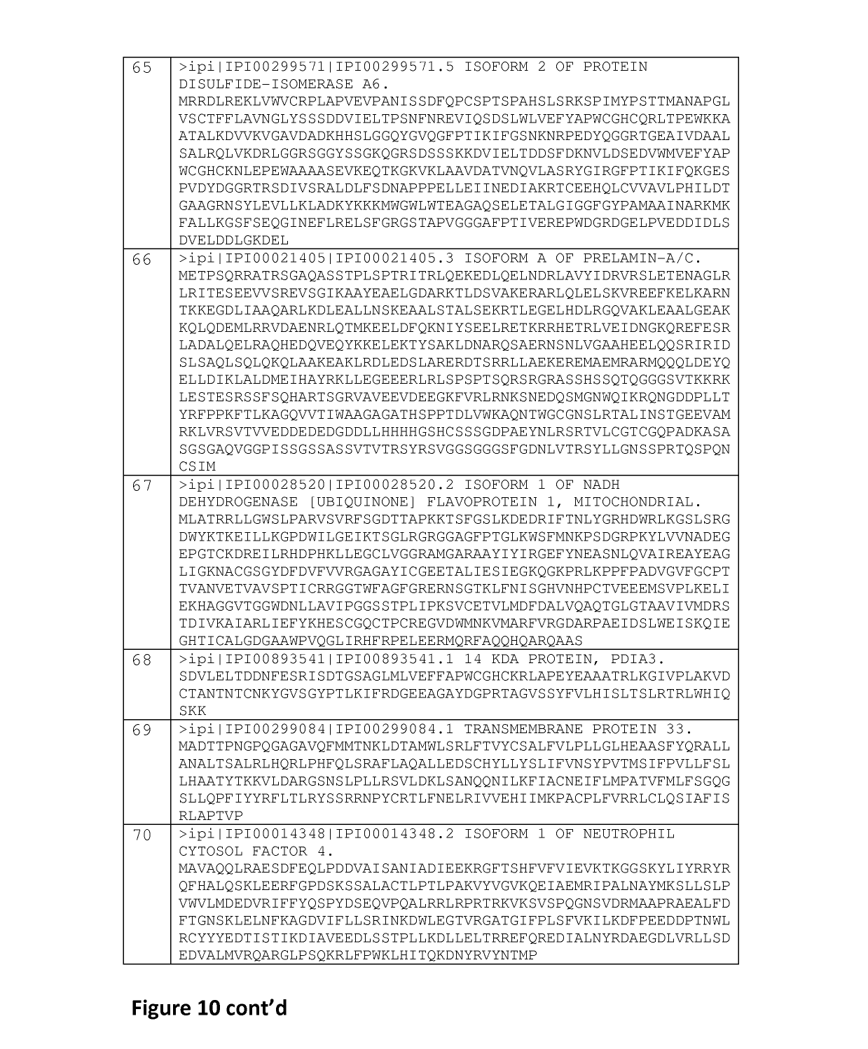

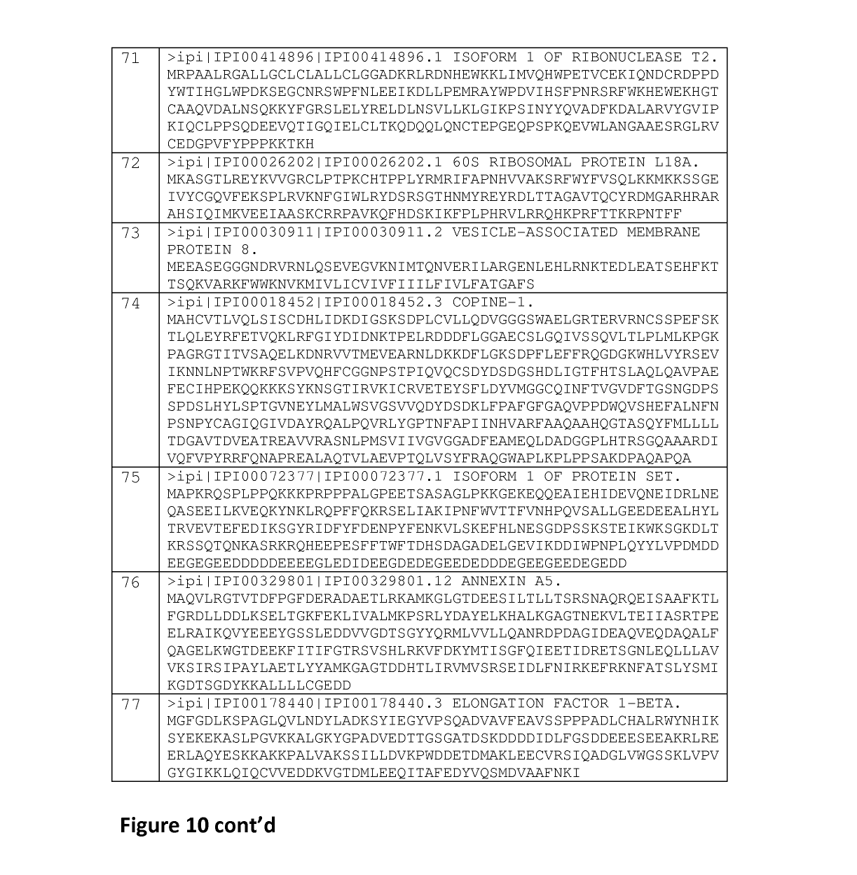

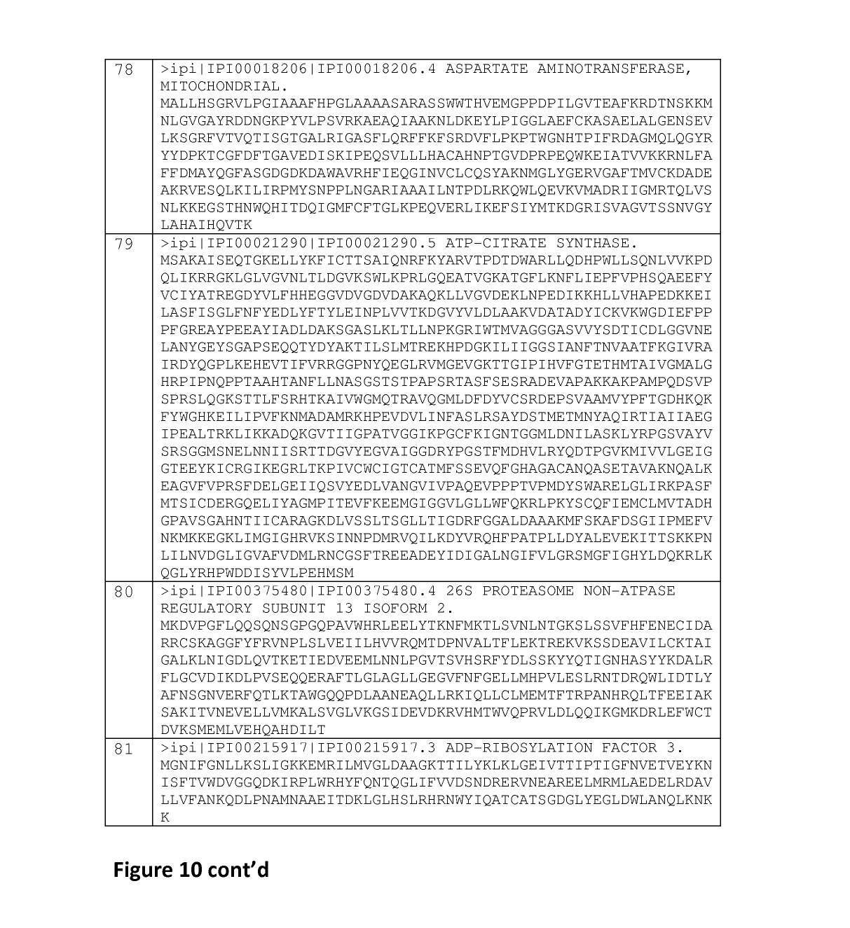

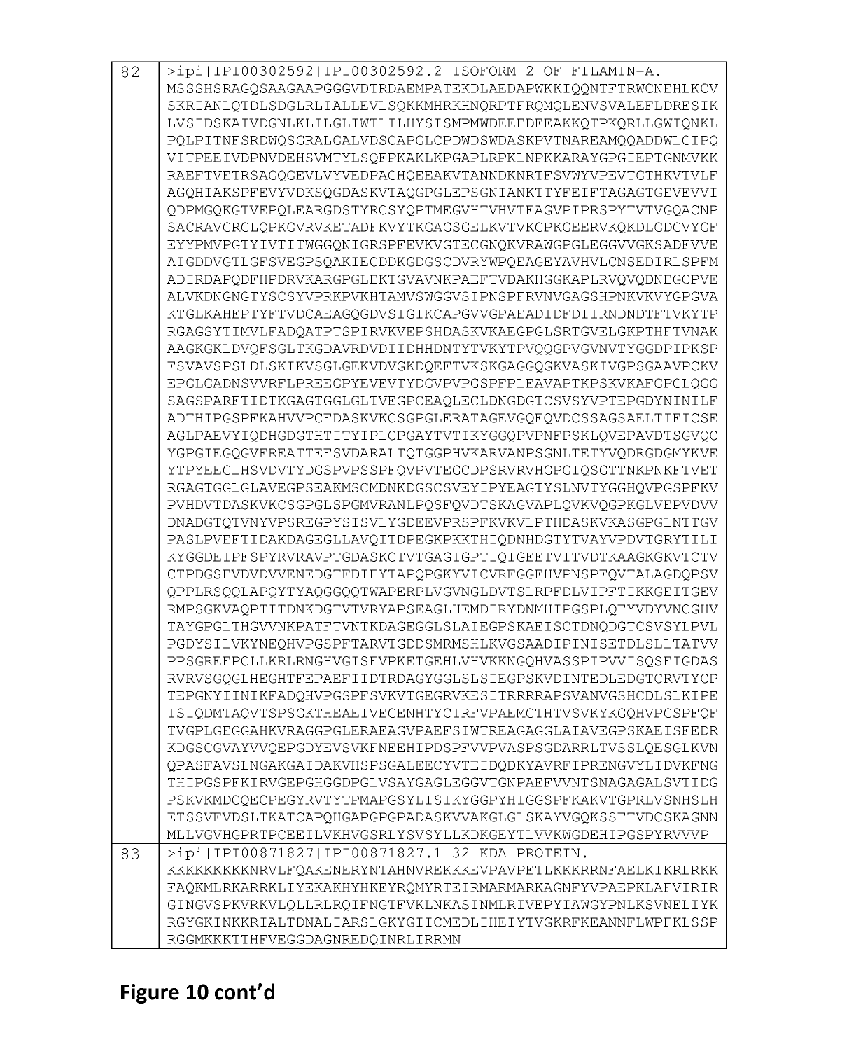

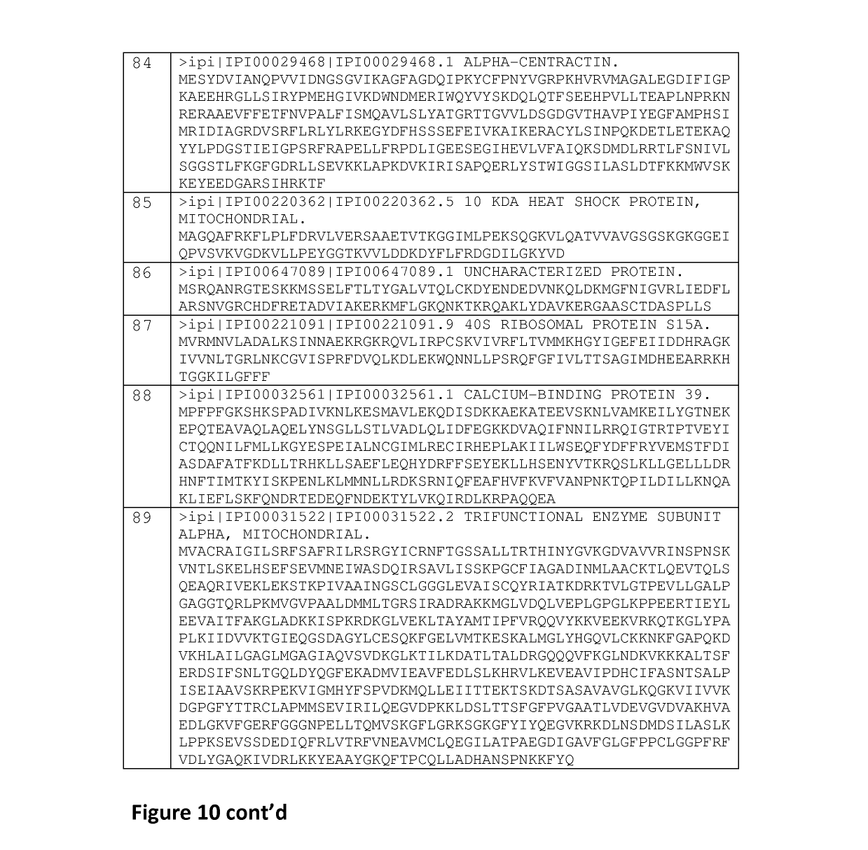

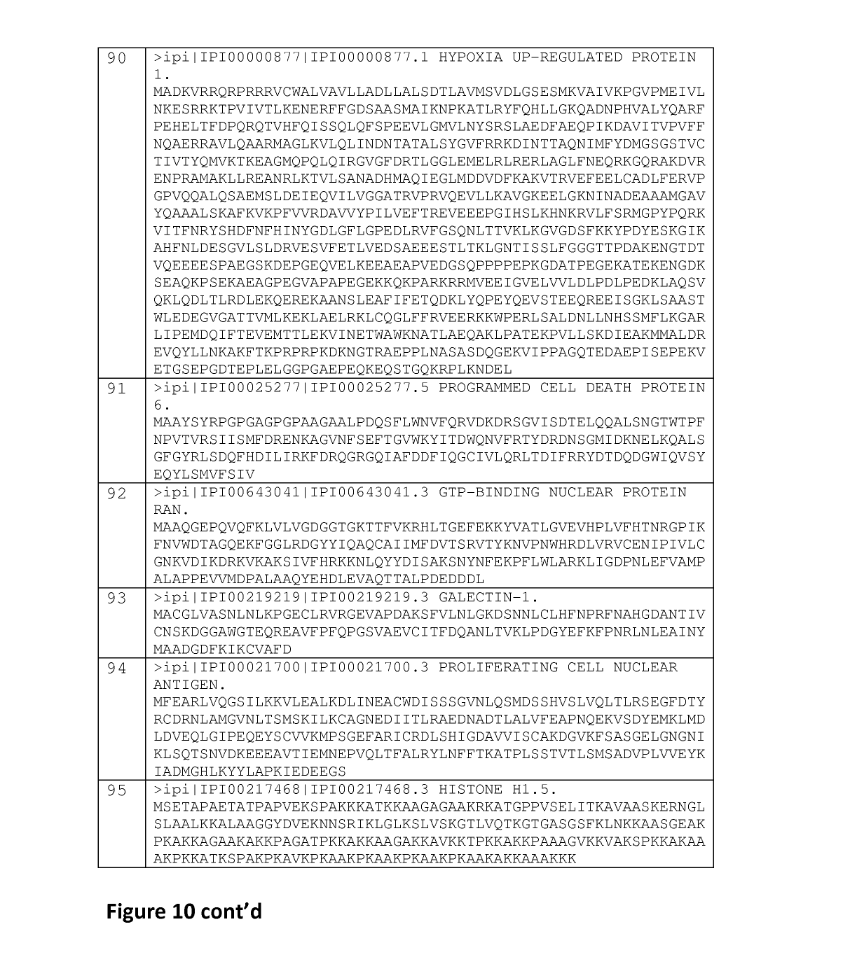

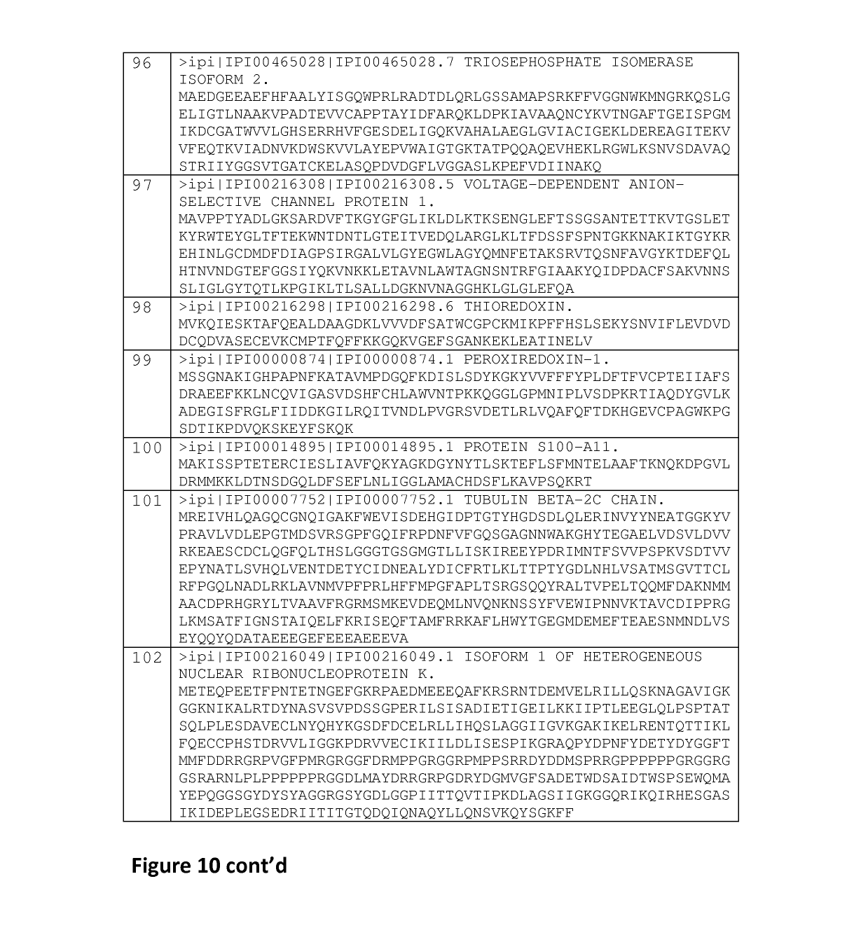

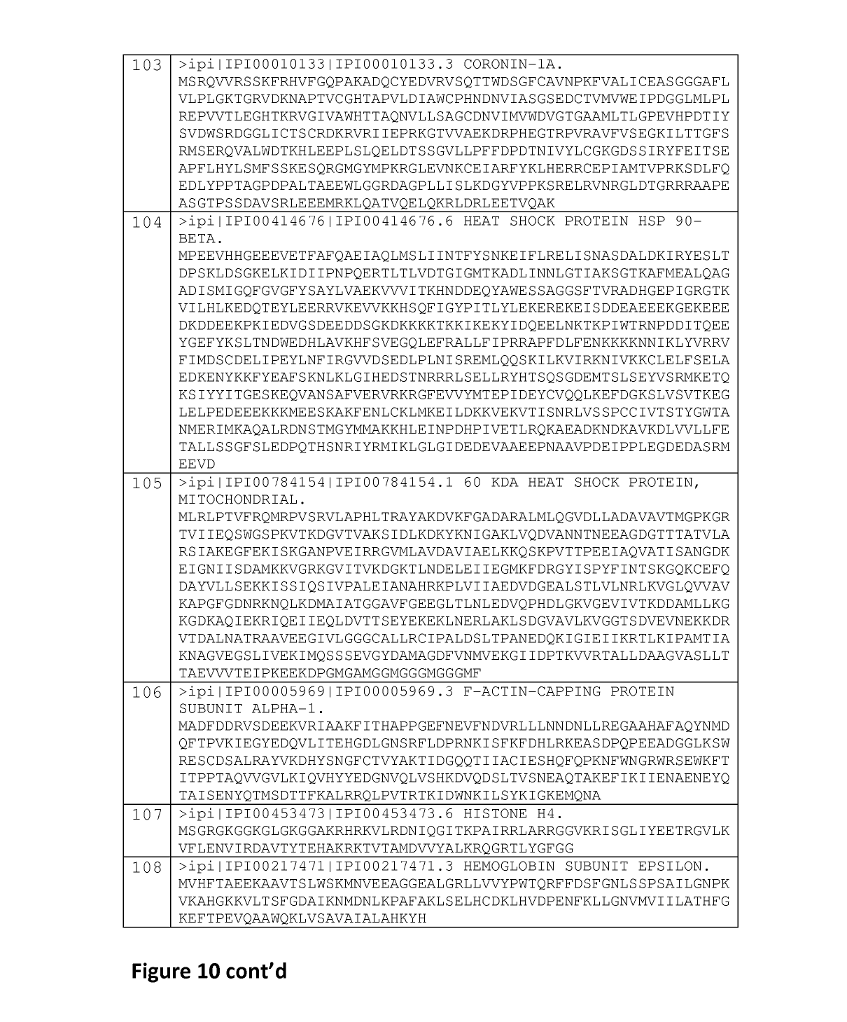

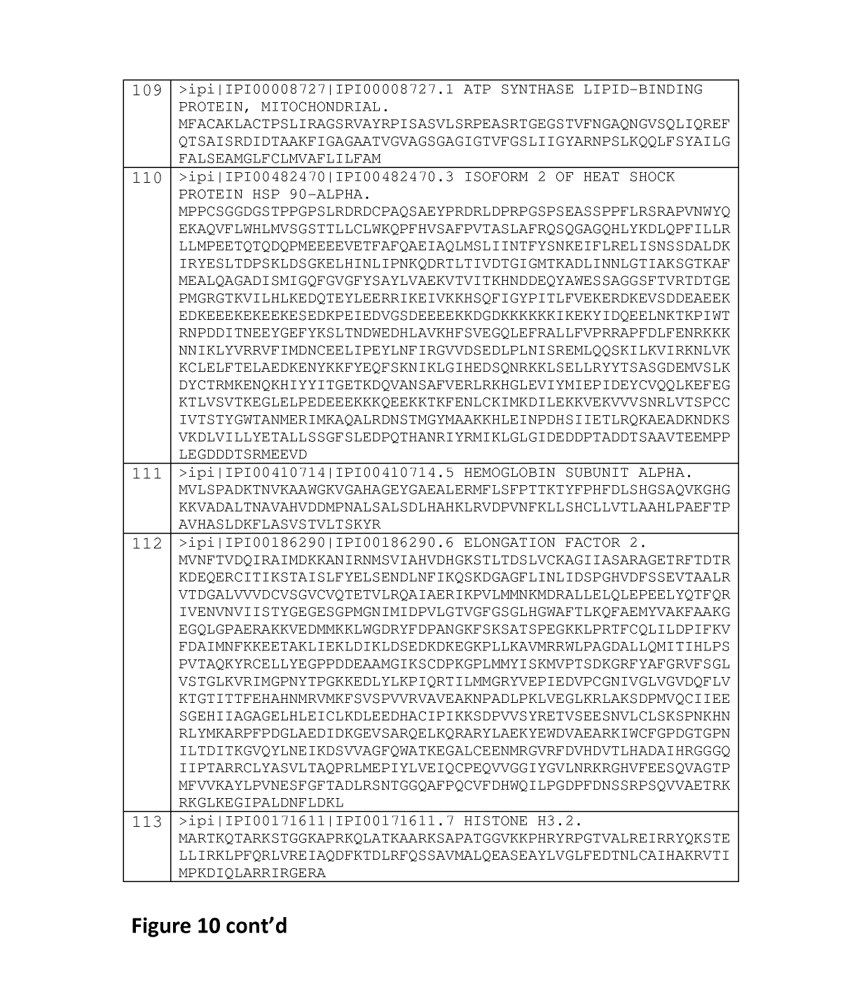

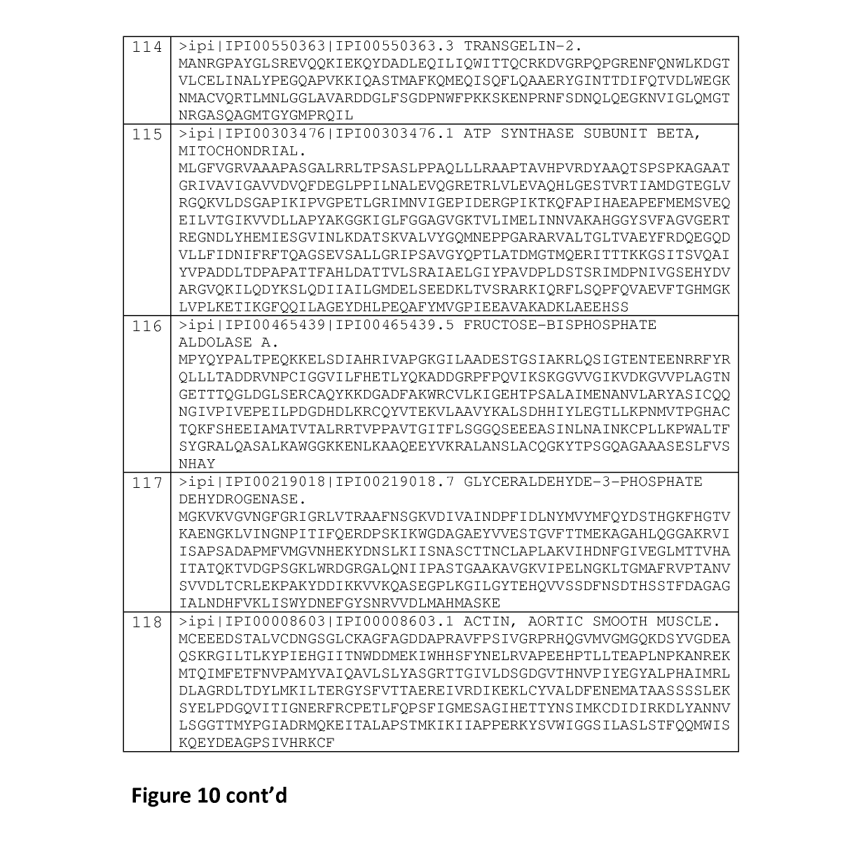

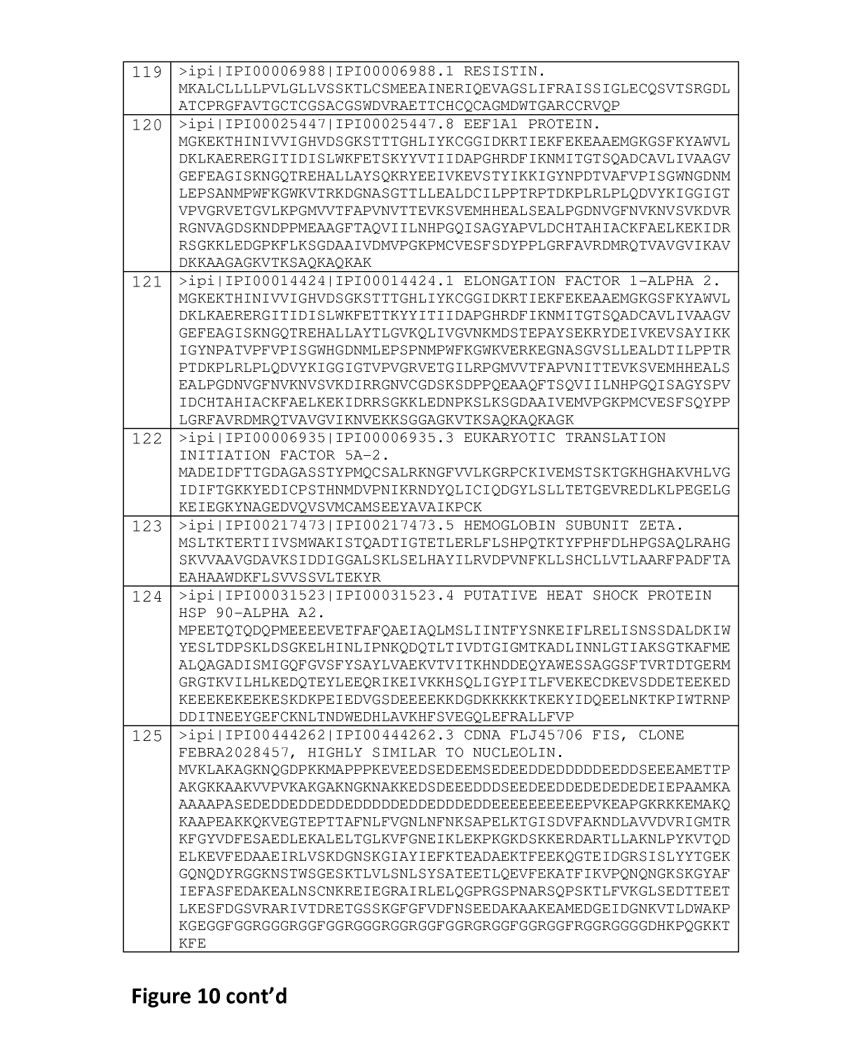

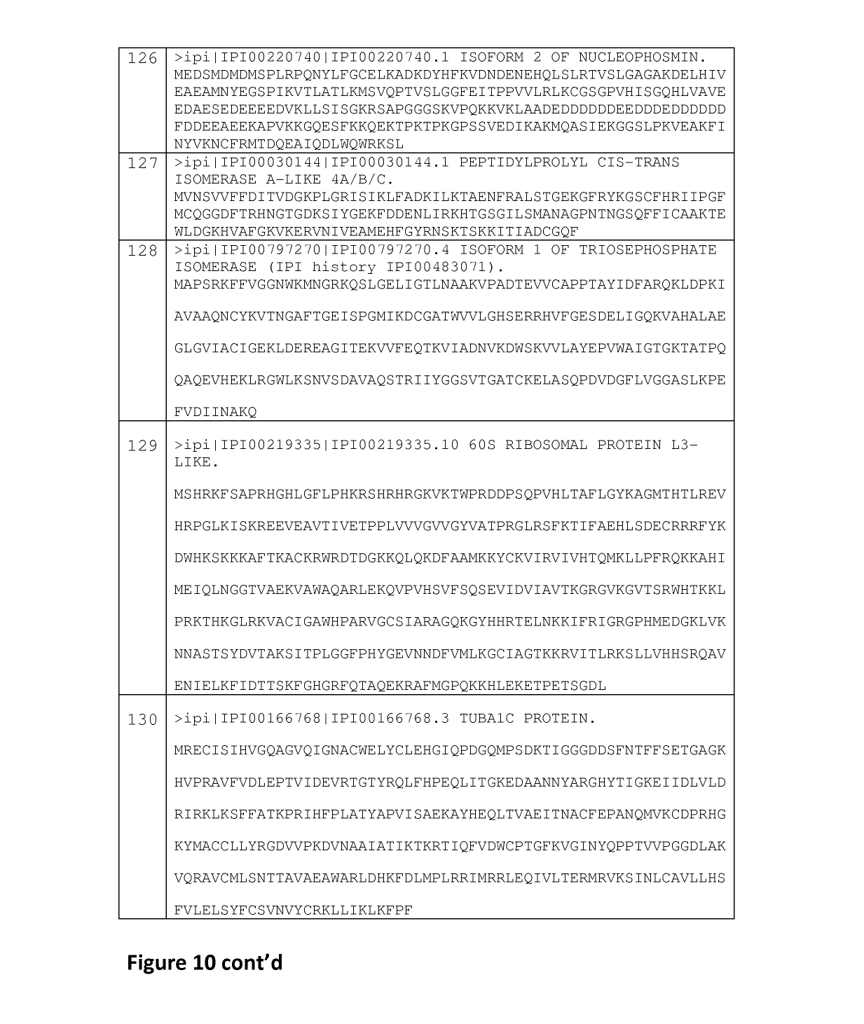

For multiple hypothesis testing an analysis of variance (ANOVA, p<=0.05) was computed to investigate biomarkers related to any of the possible contrasts between the three classes. A post hoc analysis (Tukey Test) was performed to investigate the class differences individually. The statistical scripting language R or the data analysis software MeV (TIGR) version 4.3 was used for all statistical analyses. Thereafter, a list of 130 protein biomarker was obtained (SEQ ID 1 to 130). The list is detailed in Table 1 and in the protein sequence Table 4 (FIG. 10).

### Identification of Candidate Protein Biomarkers for the Assay

To discover candidate protein biomarkers that are allow discriminating between sensitizer and non-sensitizer four discovery studies comprising different sets of chemicals listed in Table 2 were performed as described below. In the first study (40-002_{—}2) Mutz-3 cells were incubated with 2 respiratory sensitizer (150 μM MDI, 500 μM TMA), 4 contact sensitizer (10 μM DNCB, 200 μM cinnamic aldehyde, 150 μM PPD, 500 μM eugenol), 2 irritants (500 μM salicylic acid, 500 μM phenol) and 2 controls (untreated, 0.1% DMSO).

In the second study (40-002_{—}3) Mutz-3 cells were incubated with a two different concentrations of 2 contact sensitizer (50 and 100 μM cinnamic aldehyde, 4 and 8 μM DNCB), 1 irritant (300 and 600 μM SDS) and 2 controls (untreated, 0.1% DMSO). In the third study (40-002_{—}4) Mutz-3 cells were incubated with 4 contact sensitizer (120 μM cinnamic aldehyde, 4 μM DNCB, 75 μM PPD, 250 μM Oxazolone), 3 irritant (200 μM SDS, 500 μM salicylic acid, 500 μM phenol) and 2 controls (untreated, 0.1% DMSO).

In the fourth study (40-002_{—}8) Mutz-3 cells were incubated with 2 contact sensitizer (120 μM cinnamic aldehyde, 4 μM DNCB), 1 respiratory sensitizer (150 μM TMA), 1 irritant (200 μM SDS) and 1 controls (untreated).

The aim of the statistical analysis was to develop a classification model which allows assignment of a chemical into the group of sensitizing chemicals (A=allergen) or non-sensitizing chemicals (I=irritant). After statistical analysis of the four data sets (p<=0.05) a final list of candidate biomarkers was obtained. Each of the four data sets involved testing of different combinations of chemicals selected from the list of training chemicals. Thus, the use of the methods of the present invention for assessment of new chemicals or analyzing different combinations of chemicals will contribute to a growing database of biomarker candidates. In a first approach the most appropriate candidates were top-down ranked based on increasing p-value. Table 1 shows the top 130 candidate biomarkers that were significantly influenced after exposure of Mutz-3 to a set of training chemicals. Consequently Sensitizer (A=allergen) and non-sensitizer (I=irritant) can be identified by measuring the abundance of a very limited set of gene products expressed in DC or DC-like cell models:

ACLY, ACTA2, ACTN4, ACTR1A, ACTR3, AIMP1, ALDOA, ANXA5, ARF3, ARL6IP5, ATP5B, ATP5G3, BAT1, BYSL, CAB39, CALR, CAPG, CAPZA1, CLC, CORO1A, RBM12, CRTAP, EEF1A1, EEF1A2, EEF1B2, EEF2, IF3E, EIF4A3, EIF5A2, ELAVL1, FDXR, FERMT3, FLNA, G6PD, GAPDH, GOT2, HADHA, HBA2, HBE1, HBZ, HIST1H1B, HIST1H1C, HIST1H2BL, HIST2H3A, HIST1H4C, HMGN2, HNRNPK, HSP90AA2, HSP90AA2, HSP90AB1, HSPA8, HSPA9, HSPD1P6, HSPE1, HYOU1, KHSRP, LGALS1, LMNA, LRPPRC, MDH2, MPO, COX3, NARS, NASP, NCF4, NCL, NDUFV1, NME2, NPM1, P4HB, PCNA, PDCD6, PDIA3, PDIA6, PEBP1, PGD, PKLR, PPIAL3, PPIAL4C, PPP2CA, PRDX1, PSMA7, PSMC3, PSMD13, PSME1, RALY, RAN, RCTPI1, RETN, RNASET2, RPL18A, RPL26P33, RPL3L, RPL7P20, RPS15AP12, RPS19, RPS2P17, S100A11, S100A4, S100A8, S100A9, SEC22B, SERPINB1, SET, SFRS2, SFRS7, SH3BGRL3, SLC25A5, SLC3A2, SOD1, STK24, TAF15, TAGLN2, TALD01, TFRC, TMEM33, TMSL3, TPI1, TRAPPC3, TUBA1C, TUBA4A, TUBB2C, TUFM, TXN, TXNDC5, UQCRC2, VAMP8, VDAC1P1, VDAC2, VIM

In a second approach proteins were selected based on their capability to discriminate between allergen (A) and irritant (I). Post-hoc pairwise group comparisons were performed using a Tukey test. C-A: group comparison control versus allergen; I-A: group comparison irritant versus allergen; I-C: group comparison irritant versus control.

In the first study the comparison between allergen and irritant identified 17 proteins (Tukey test p<=0.05): ATP5B, MPO, S100A11, ACTA2 HBA2/HBA1, S100A8, NCL, VDAC1, EEF1A2, PPIA, NPM1, TMSL3, TXN, HBE1, TUBA4A, HNRNPK and PPIAL4.

In the second study the comparison between allergen and irritant identified 7 proteins (Tukey test p<=0.05): TUBA4A, HSP90AA1, PKLR, CLC, HSP90AA2, TALD01 and VDAC2.

In the third study the comparison between allergen and irritant identified 12 proteins (Tukey test p<=0.05: HIST1H1C, CLC, HIST2H3D, RCTPI1, HIST1H1B, TMSL3, HIST1H2BL, GAPDH PCNA, HIST2H4A; SLC25A5 and PRDX1.

It is also possible to select the most promising biomarker candidates by analyzing the overlap between the different data sets. In a third approach candidate protein biomarkers were selected if the same protein was found in 2 out of 4 of the different data sets (p<=0.05) which involved testing of different combinations of chemicals. These proteins belong to a set of proteins comprising the 15 most promising proteins. Group 1: contains the following proteins: HSPA8, MPO, S100A8, TMSL3, VDAC2, CLC, HIST1H1C, HIST1H2BL, PGD, PKLR, PPIA, S100A9, SLC25A5, TALD01 and TUBA4A, that overlapped in at least 2 of 4 experiments (p<=0.05).

### Example 3

### Biomarkers for Respiratory Sensitizer

Currently, there is a lack of validated cell-line based assays for identifying respiratory sensitizers and the IL-8 assay in dendritic cell lines fails to respond to TMA (Mitjans et al. 2009). To identify appropriate protein biomarkers for the respiratory sensitizers Mutz-3 cells were exposed to reference compounds including a respiratory sensitizer (TMA), a prototypic contact sensitizer (DNCB), one irritant (SDS) or were left untreated (fourth study). A two sample t-test (p<=0.05) comparing TMA versus control samples was performed to identify biomarkers that are indicative of cellular response to the respiratory sensitizer TMA. Proteins were ordered by increasing p-value.

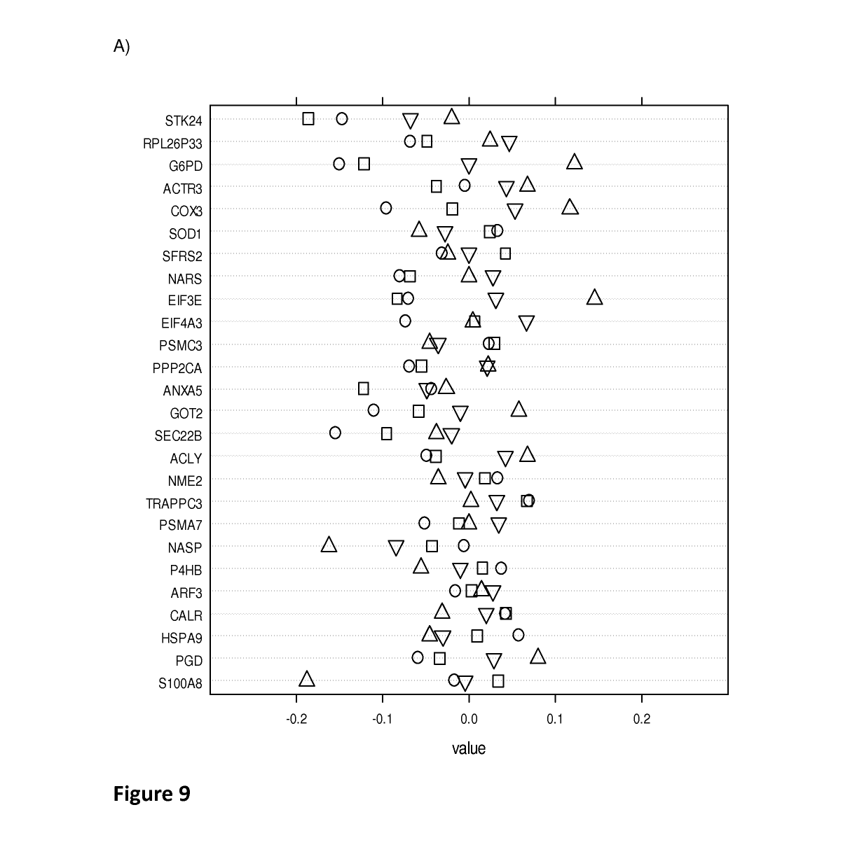

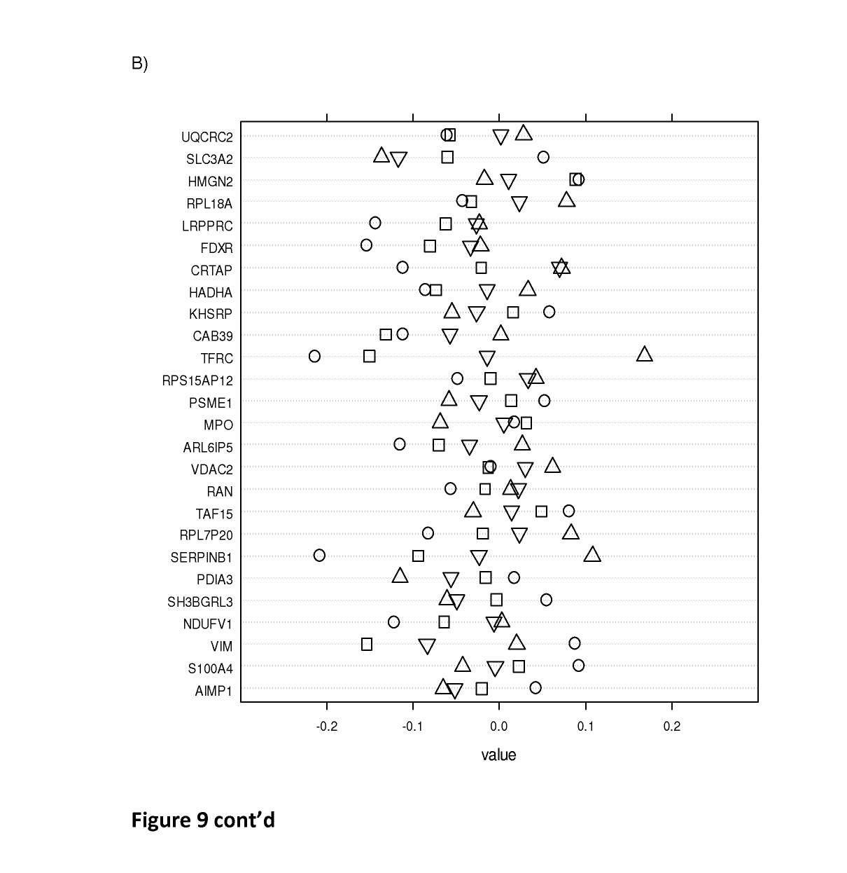

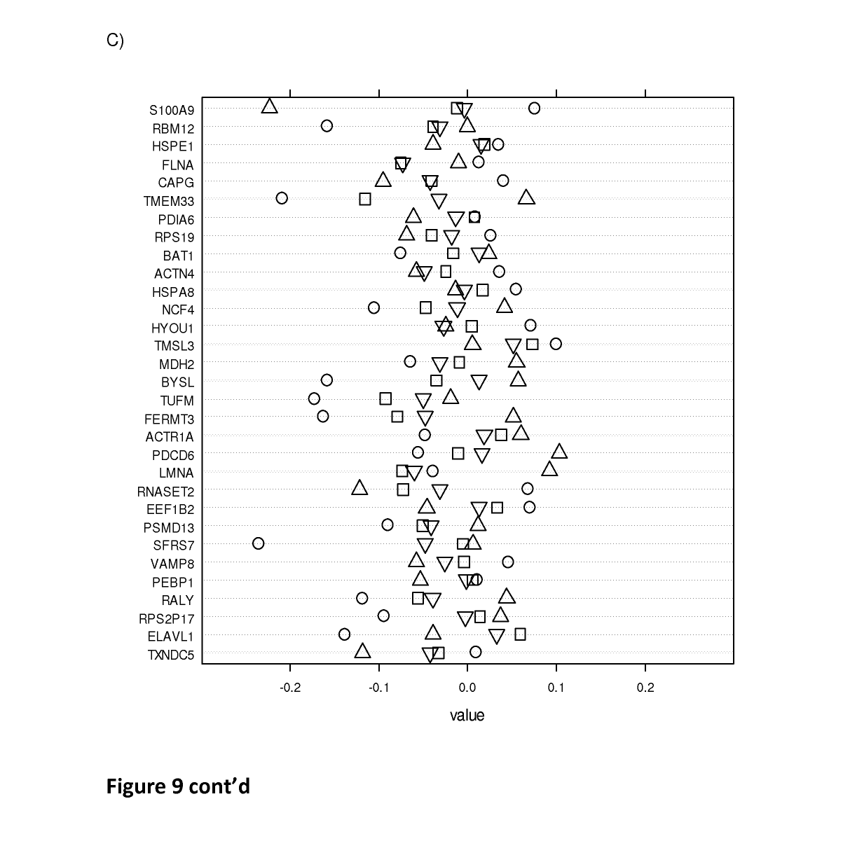

Proteins in Group 2 of Table 1 represent a TMA-specific marker panel comprising ACTR3, EIF3E, G6PD, COX3, NARS, RPL26P33, SFRS2, EIF4A3, SOD1, STK24. Table 1 gives the uniprot ID, the protein name and official gene name of the biomarkers. The respective protein sequences are shown in Table 4 (FIG. 10). Differences in the relative abundance of the candidate biomarkers between control, irritant, TMA and a typically skin sensitizer DNCB is shown in FIG. 9.

Group 3 of Table 1 contains further sensitizer biomarkers that pass the required significance criteria of p<=0.05 in at least one of the four studies that involved testing of different combinations of chemical irritants and sensitizers.

### Hierarchical Cluster Analysis

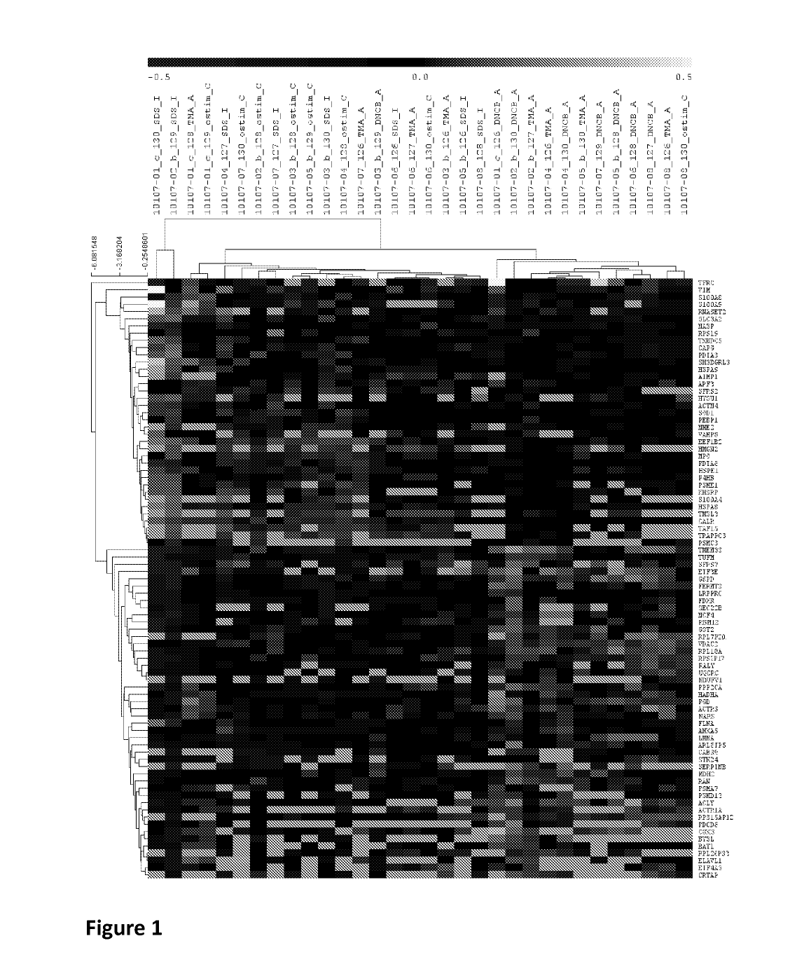

FIG. 1 shows another example of the utility of the proposed biomarkers for discriminating sensitizer and irritants. In the fourth study Mutz-3 cells were exposed to one contact sensitizer (DNCB), one respiratory sensitizer TMA, one irritant (SDS) or were left untreated. A subset of the proteins from the list of interest comprising 83 proteins were used for an unsupervised hierarchical clustering and showed good segregation of samples belonging to the group of allergen, irritant and control. Two main clusters of proteins with increased or decreased abundance were found.

### Partial Least Squares-Discriminant Analysis (PLS-DA)

A preferred method for classification of chemicals is employing partial least squares regression analysis (PLS). PLS discriminant analysis identifies the most important classifiers from the list of interest. A model was built using the response variables (y) highlighted in red and the ANOVA filtered proteins as predictors (x). The first PLS components (x-axis) was plotted against the second PLS component (y-axis) and separated the biomarkers having a role in classifying the three experimental groups “control”, “irritant” and sensitizer”.

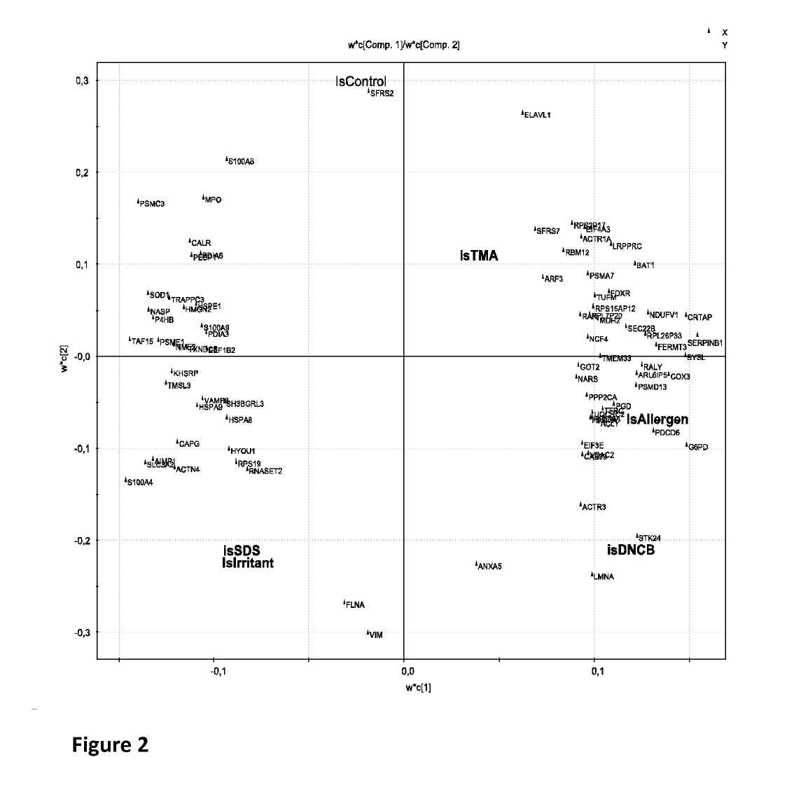

FIG. 2 shows the PLS-DA loadings plot of candidate biomarkers found in the fourth study. Protein biomarkers (IPI accession numbers) are plotted using coordinates corresponding to weight variables for the first two principal axes. Biomarkers that are close to the response variable “is allergen” have a strong role in classifying chemicals as potential allergens. Biomarkers that are close to the response variable “is irritant” identify chemicals as irritants.

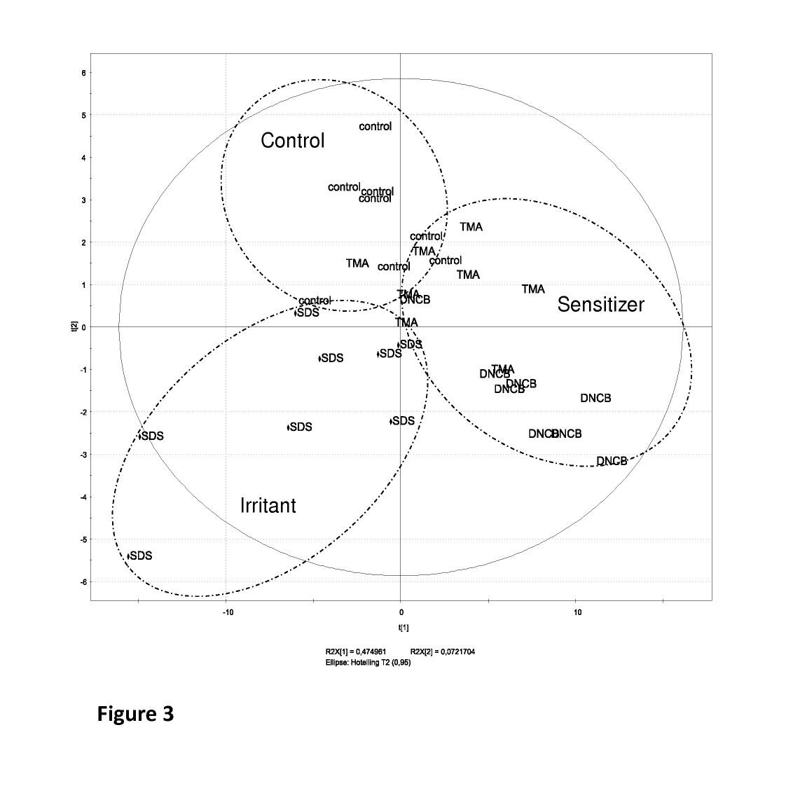

FIG. 3 shows the corresponding PLS score plot of the two first principal components that are plotted against each other for all samples in the fourth study. In the score plot each point represents one 1 sample. Based on the ANOVA filtered protein list samples a good separation between control, sensitizer and irritant treated samples was achieved. It can be seen that that samples treated with the very strong sensitizer DNCB had the greatest relative distance from the control, whereas TMA treated samples showed a smaller distance to control sample indicating that TMA elicits a much smaller response in MUTZ-3 cells.

### Example 4

### Targeted Biomarker Measurements

As an alternative approach to find predictive markers for sensitizing potential we explored the potential of cell culture supernatants as a source of markers for chemical safety by adopting a more targeted approach using commercially available immunoassays to measure the levels of three known inflammatory markers. The three proteins to be measured by ELISA were selected on the basis of a review of literature references to secreted inflammatory protein response to chemical sensitizer. Cells were cultured as described in Example 1 and the spent medium removed for direct analysis by ELISA. All kits were used in accordance with manufacturer's instructions.

### Myeloperoxidase (MPO)

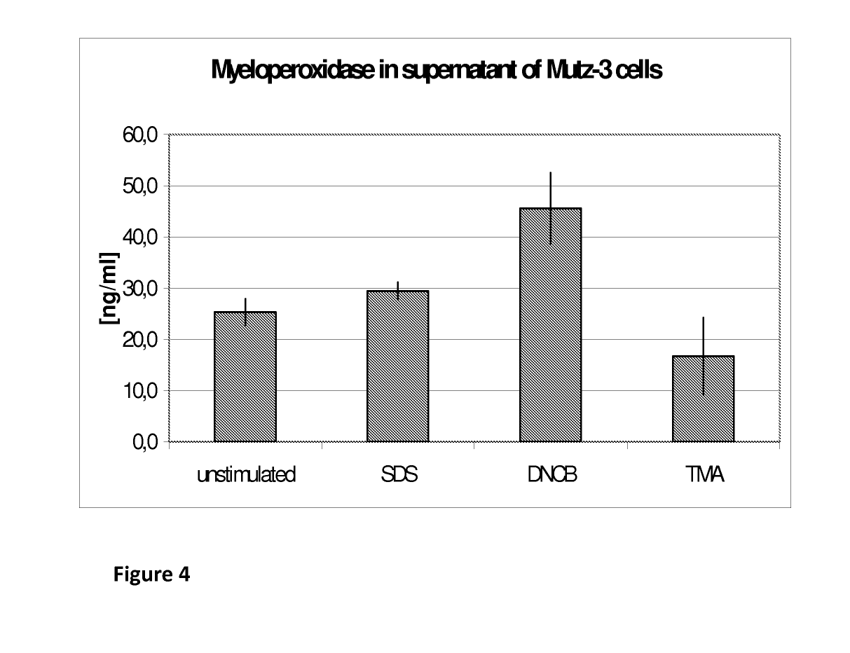

According to Hu et al. (1996) the MUTZ-3 cell line shows characteristics of monocytes and expresses MPO. MPO is a peroxidase enzyme which is stored in lysosomes and released during inflammation Myeloperoxidase was measured by ELISA (Assay-Designs) (FIG. 4). Surprisingly, the results showed that MPO is indicative of the exposure of Mutz-3 cells to the contact sensitizer DNCB, but not to the respiratory sensitizer TMA. These results indicate that the contact sensitizer DNCB activates an inflammatory pathway involving release of the peroxidase enzyme MPO, whereas the respiratory sensitizer TMA does not activate this pathway. It should be noted that in Example 1 we identified a decrease in the intracellular levels of MPO which, in combination with the increased levels of MPO found in supernatant of Mutz-3 cells suggests an active secretion of MPO in response to exposure with contact sensitizers.

### Calprotectin (S100A8/S100A9)

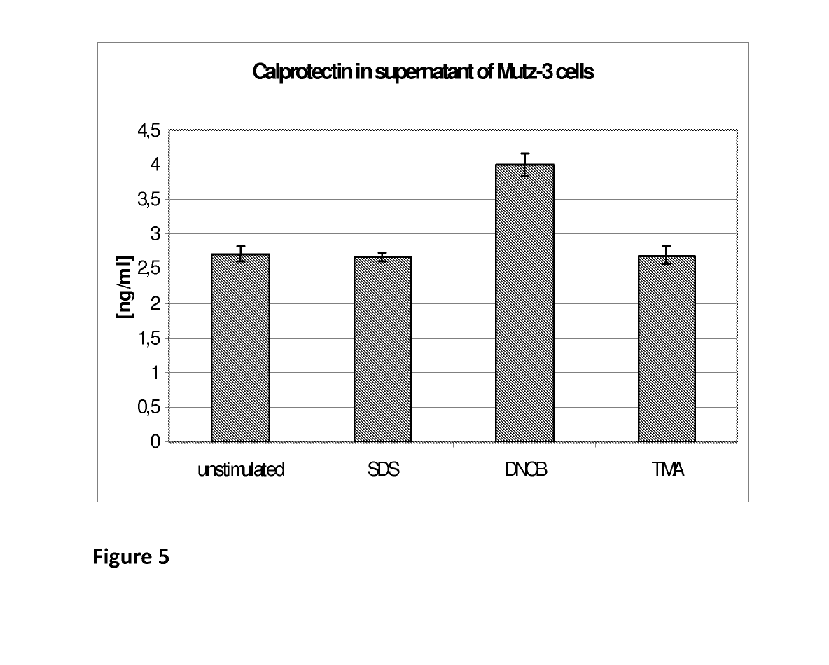

Calprotectin is heterodimer of S100A8/S100A9 was measured in supernatant of Mutz-3 cells by a commercially available immunoassay (Immundiagnostik AG, Bensheim, Germany) (FIG. 5). Calprotectin is actively secreted by phagocytes in response to stress and binds to Toll-like receptor 4 (TLR4). Toll-like receptors play an important role in the innate immune system and activation of TLR4 by calprotectin further amplifies inflammation (Ehrchen et al. 2009). TLR4 and interleukin 12 double knock-out mice, show a marked decrease of DC-sensitization by a broad range of different sensitizers including 2,4,6-trinitro-1-chlorobenzene (TNCB), oxazolone, and fluorescein isothiocyanate (Martin et al. 2008). Based on the general inflammatory pathway implicated in calprotectin secretion it might be expected to be a marker for all classes of chemical allergens. Unexpectedly, our data show that S100A8/S100A9 levels are modulated by DNCB exposure, but not in response to the respiratory sensitizer TMA indicating that different cellular pathways are activated by the two different chemicals. As with MPO, the increased extracellular levels of calprotectin were matched by a correlating decrease in intracellular levels seen in Example 1.

### Cu/Zn-SOD (SOD1)

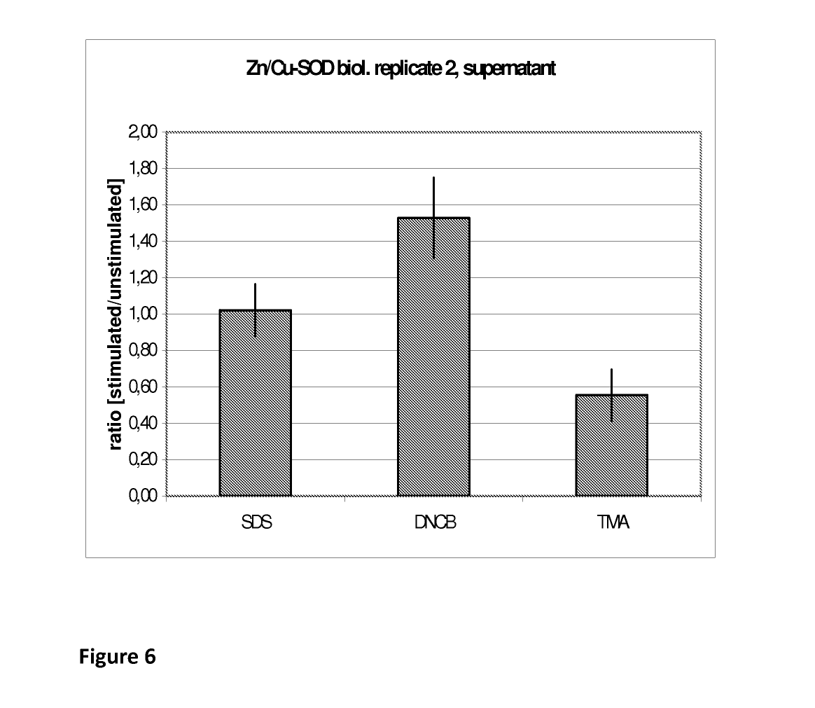

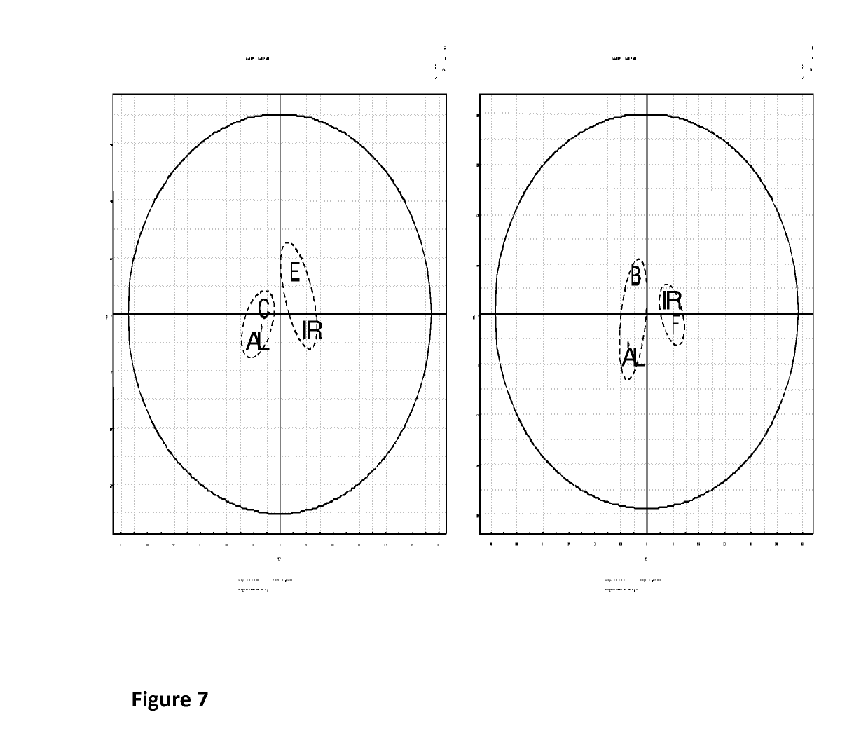

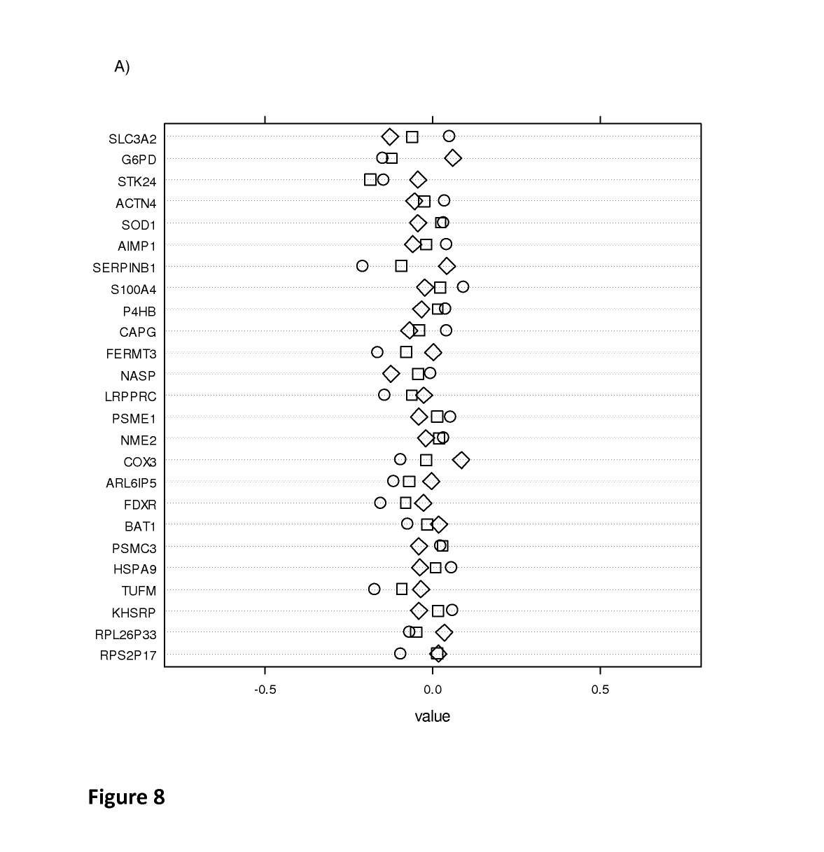

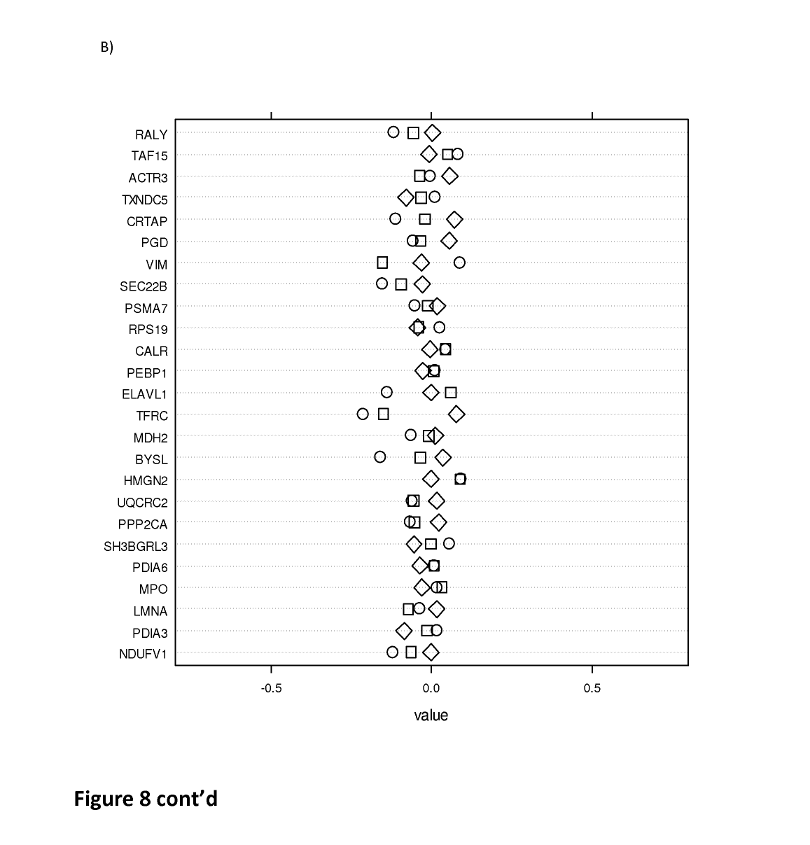

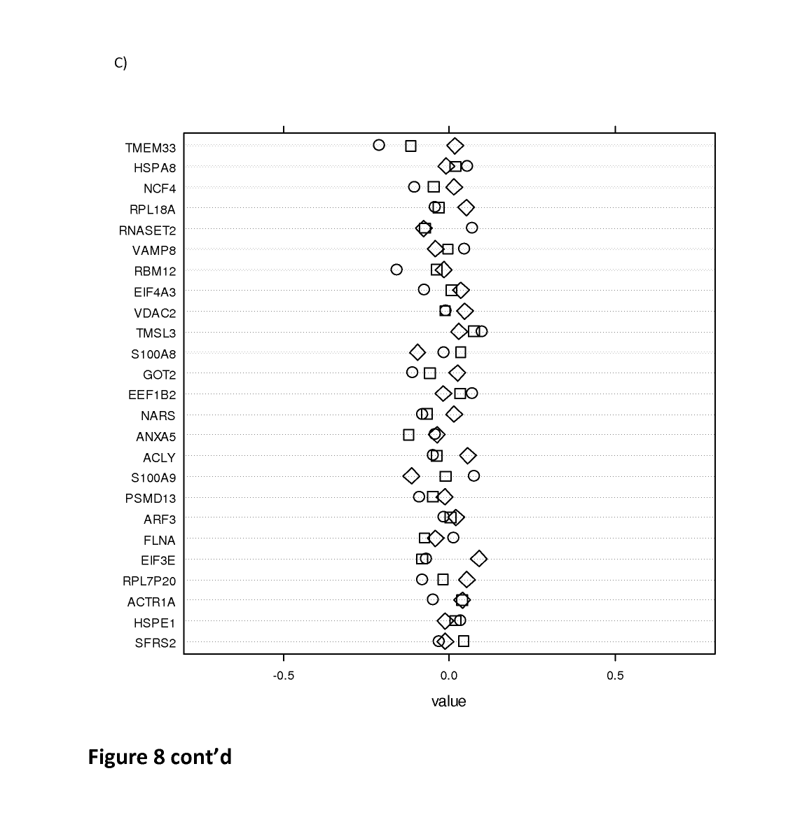

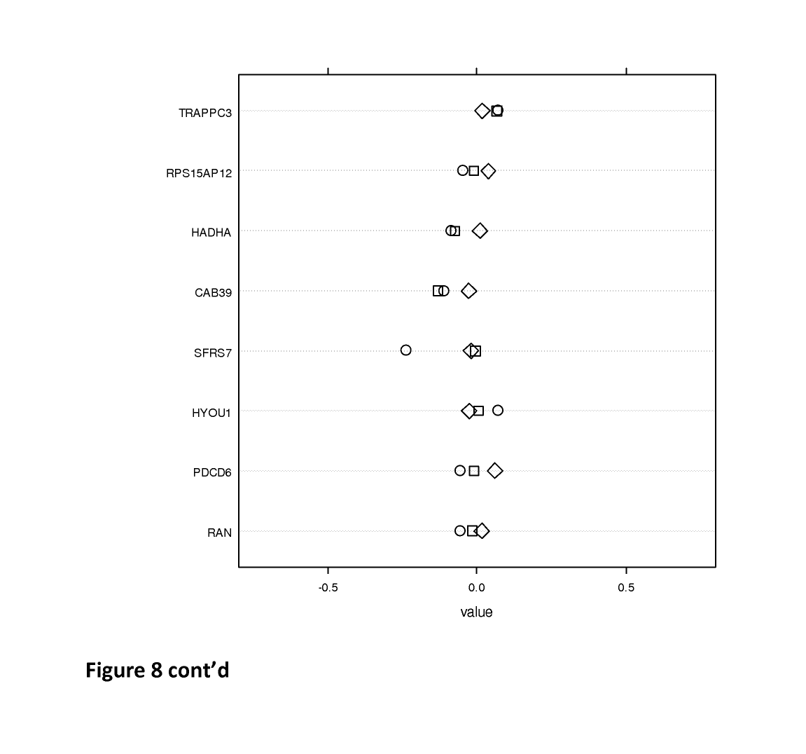

Zn/Cu-SOD (SOD1) is an enzyme that converts free superoxide radicals to molecular oxygen and hydrogen peroxide. SOD1 is an intracellular enzyme expressed in all cells of the body. During DC differentiation SOD1 expression increases and reaches highest levels in mature DCs (Rivollier et al. 2006). The SOD1 expression is upregulated by pro-inflammatory mediators such as LPS, TNF-alpha and IL-lb (Visner et al. 1990). In contrast to Rivollier et al. (2006), who reported an increase in cell associated SOD1 levels at the mature DC stage, we found that SOD1 level were decreased in response to allergen exposure in cell extracts and increased in supernatant of Mutz-3 (FIG. 6). In supernatant SOD1 was quantified using a specific ELISA assay (IBL, Hamburg, Germany).

### Example 5

### Biomarker Panel for General Sensitizers

The results of Examples 1-4 have identified a panel of 130 proteins (Table 1, Groups 1-3) for the discrimination of chemical sensitizers from irritant or control chemicals. The method described to determine the panel of 130 biomarkers using reference chemicals can now be used in a revised form to test new or previously untested chemical agents to determine their potential as allergens, sensitizers or non-sensitizing. According to the present invention the method may employ measuring the concentration of biomarkers chosen from table 1 in test sets of samples exposed to new chemicals or new chemicals in combination with reference compounds as positive and negative controls. Typically, when evaluating a new test chemical the analysis should be performed using a combination of biomarkers from Table 1, especially selecting biomarkers from group 1 or group 2. Use of a panel of biomarkers selected from group 1 and group 2 will ensure the most robust discrimination between sensitizer, irritant and control. When the selected biomarkers perform well on a new chemical compound one would retain the combination of biomarkers. Alternatively it is possible test other combinations of biomarkers from group 1 or 2 in an iterative process. It is also possible to reject biomarkers from group 1 or 2 and include biomarkers from group 3. This iterative process will continue until a good classification model is produced. It is also possible to identify chemicals causing skin irritation by measuring the concentration of biomarkers detailed in Table 1. Typically, when discriminating between potential irritants and sensitizers, the analysis should be performed using biomarkers linked to inflammatory and cellular stress processes. In particular, biomarkers selected from group 3 that are induced following exposure to SDS may be useful. This may involve but is not limited to measuring the concentration of SLC3A2, a component of the transmembrane glycoprotein CD98, or HYOU1 a protein having an important cytoprotective role in hypoxia-induced cellular perturbation or SH3BGRL3/TIP-B1, a TNF inhibitory protein. The inclusion and combination of proteins modulated by sensitizing or irritating chemicals will allow to correctly predict the sensitizing or irritating potential of new chemicals.

### Example 6

### Biomarker Panel for Contact Sensitizers

Within the panel of general markers of sensitizing potential it is also possible to select the strongest discriminant markers correlating with contact sensitizer effect. Using PLS-DA a sub-group of 15 proteins providing the strongest separation of skin sensitizers from all other classes was identified (Table 1, Group 1). It was thus possible to perform a targeted analysis to measure just these 15 proteins to detect known skin sensitizers and demonstrate whether an unknown test chemical or combination of chemicals possesses skin sensitizing potential.

### Example 7

### Biomarker Panel for Respiratory Sensitizers

Within the panel of general markers of sensitizing potential it is also possible to select the strongest discriminant markers correlating with respiratory sensitizer effect. Using PLS-DA a sub-group of 10 proteins providing the strongest separation of respiratory sensitizers from all other classes was identified (Table 1, Group 2). It was thus possible to perform a targeted analysis to measure just these 10 proteins to detect known skin sensitizers and demonstrate whether an unknown test chemical or combination of chemicals possesses skin sensitizing potential.

### Example 8

### Method of Determining Allergic and Irritant Potential of Unknown Chemicals

We used the MUTZ-3 culture dendritic cell model to test a range of compounds whose allergic or irritant status was unknown at the time of testing. MUTZ-3 cells were cultured as described in Example 1 and exposed to five unknown chemicals (A, B, C; E, F). On average three samples were treated per compound Cultures of MUTZ-3 cells were also incubated with the known allergen PPD and the known irritant SDS or were left untreated to serve as positive and negative controls respectively. After cell culture, the spent medium was removed and stored for future analysis. Cells were washed and harvested and proteins extracted and labeled with TMT as described in Example 1.

Labelled lysates for each unknown compound mixed with allergen, irritant and non-sensitizer controls and a reference cell digest and subjected to LC-MS/MS analysis essentially as described in Example 1. Following mass spectrometry, data was assembled and the TMT reporter ion spectra for the 130 defined markers in Table 1 were extracted and used to quantify the relative abundance of each protein in test and control samples. To construct a first test that assigns the unknown chemical to a particular category, it is possible to employ mathematical linear regression models. A straightforward model is PLS-DA in which the known sensitizer and irritant serve as internal reference points to predict the particular chemical class of an unknown chemical from the level of the protein predictor variables. Such a model was employed to analyse individual marker performance in the blinded study based on the previous findings of key discriminant markers for sensitizing, irritant and control chemicals. The results from the unknown compounds used for testing were then un-blinded and their true status compared with the actual status. In four of five cases the quantitative protein biomarker panel allowed the correct assignment of chemical safety status to the unknown compounds. The results of this first PLS-DA analysis for the four correctly identified chemicals are shown in FIG. 3. The PLS score plots represent individual measurements in which two unknown chemicals were tested against PPD as a positive and SDS as a negative control. Based on biomarkers selected from Table 1 a short distance to the reference chemical indicates that these chemicals share some common features. The sensitizing potential of these chemicals was determined before using the murine Local Lymph Node Assay (LLNA) or Guinea Pig skin reaction test. Based on the LLNA Ethylendiamine (chemical A) is typical pro-hapten which requires characterized as a moderate sensitizer, but because it is viewed as a typical pro-hapten false negative results are frequently obtained using cell culture based tests (Natch 2010). The category of chemical A could not be predicted. Ethyl vanillin (chemical B) is classified as extremely weak and non-sensitizing, but has been positively tested in the Guinea Pig skin test. Using the list of biomarkers from Table 1 chemical B (ethyl vanillin) was classified as a weak sensitizer. Chemical C (formaldehyde) was correctly classified as strong sensitizer and chemicals E (Isopropanol) and F (Methyl salicylate) as non-sensitizer, respectively.

TABLE 3
Test chemicals and outcome of the prediction

| Chemical | LLNA | Prediction |
|---|---|---|
| A = Ethylene diamine | Moderate | False negative |
| B = Ethyl vanillin | Extremely weak/ | Weak sensitizer |
| | non-sensitizing | |
| C = Formaldehyde | Strong sensitizer | Strong sensitizer |
| E = Isopropanol | Non-sensitizer | Non-sensitizer |
| F = Methyl salicylate | Non-sensitizer | Non-sensitizer |

### Example 9

### Selective Reaction Monitoring (SRM) Assays for a Panel of Biomarkers Relevant for Specific Pathways

To approach the complex and variable cellular response to different chemical sensitizers, assays are required that allow simultaneous analysis of several biomarkers representing different cellular response pathways. SRM-based approaches are an attractive alternative to ELISAs due to the sensitivity and selectivity of the technique, the capacity to multiplex and the limited availability of antibodies. Here, signature peptides unique to the protein of interest are measured to provide quantitative information of that protein in the sample. Changes in peptide abundance in response to chemical exposure experiments can be determined using typical isotopic TMT-SRM workflows. Here, quantitation is based on the relative MS intensities of the sample peptide labelled with TMTzero versus an internal reference sample labelled with TMTsixplex heavy isotope.

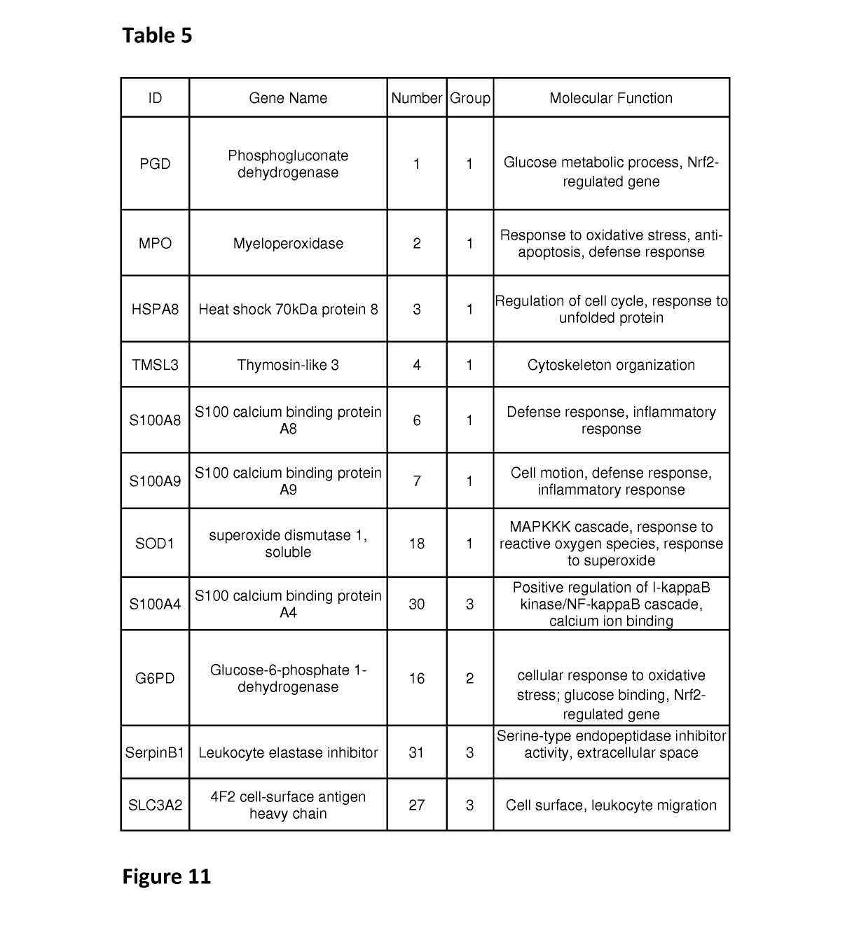

Table 5 (FIG. 11) shows biomarker candidates emerging from the discovery data that were selected for assay development based on statistical significance and pathway representation.

### Methods

### Selection of Candidate Peptides for SRM Quantitation

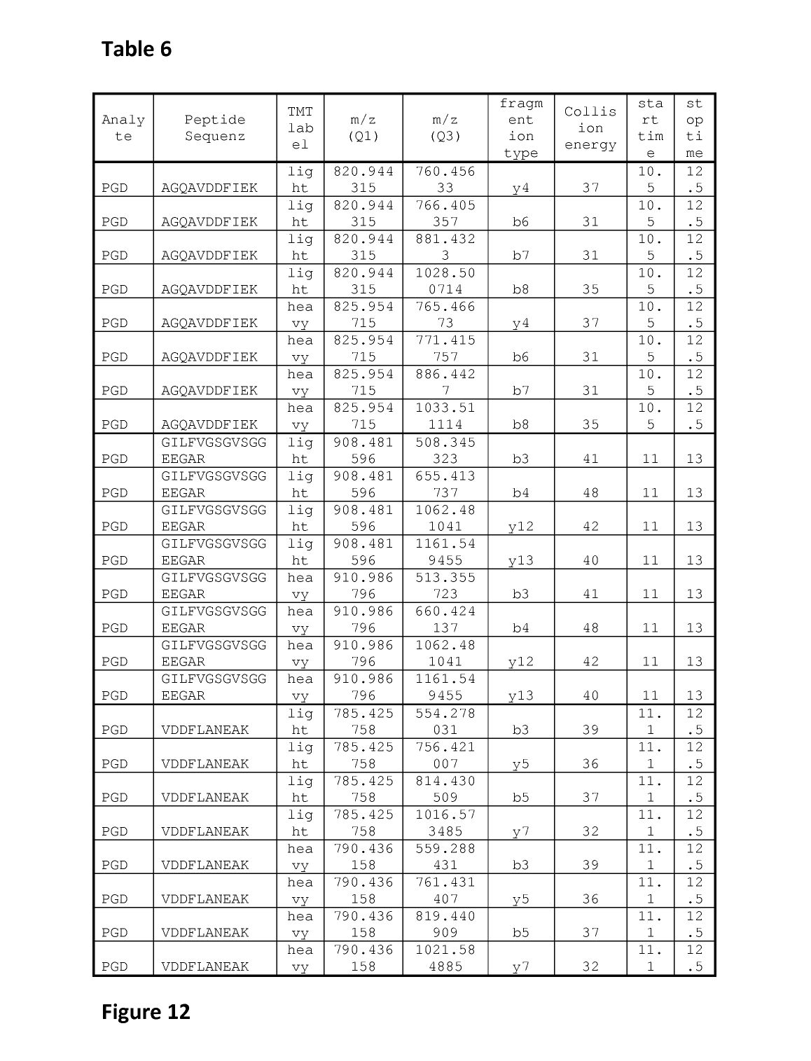

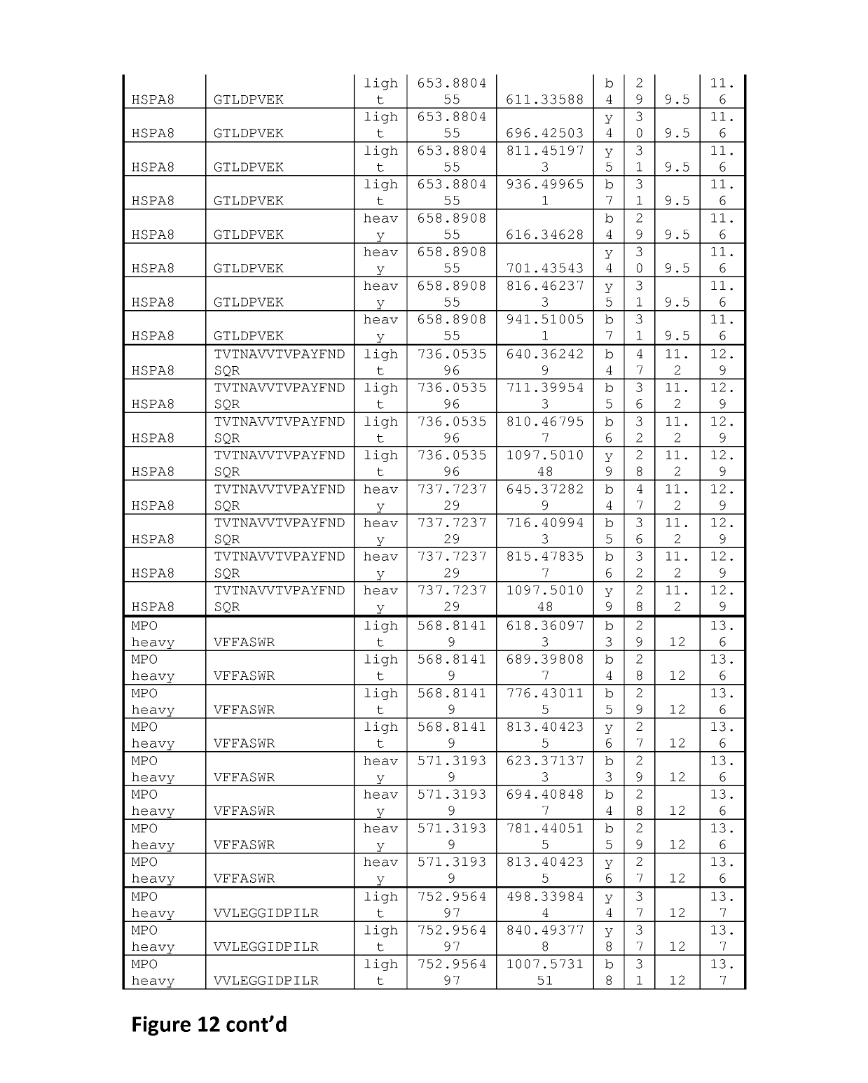

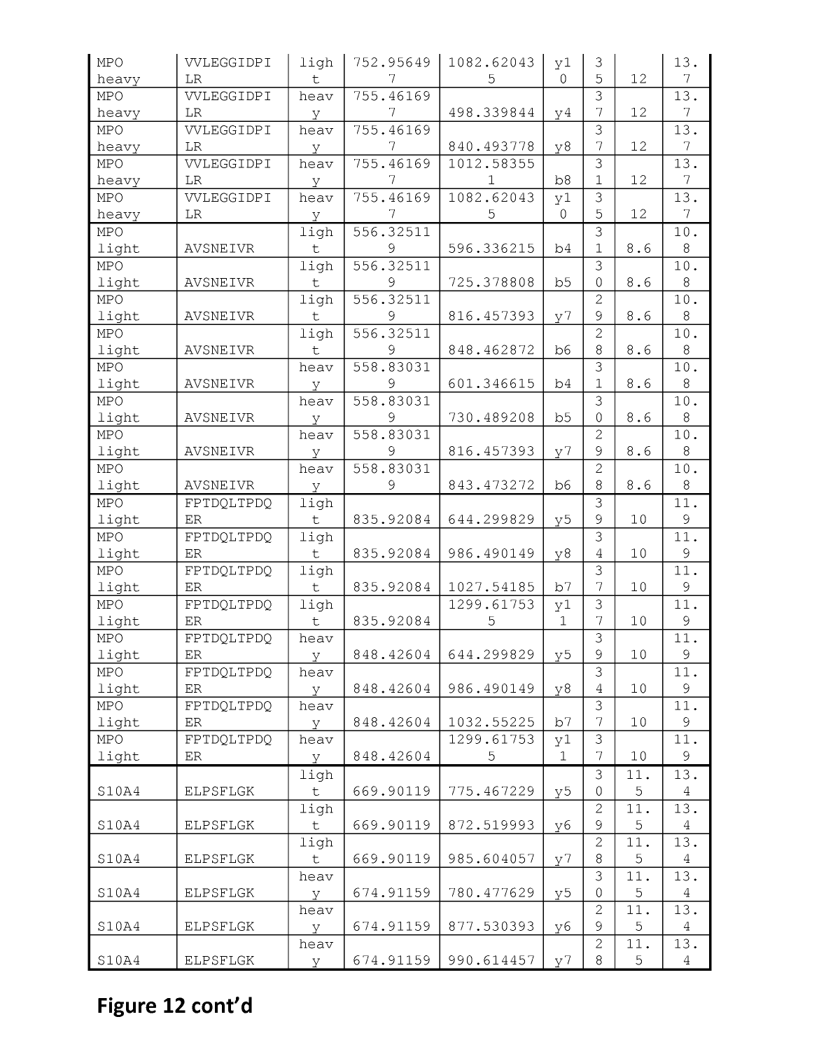

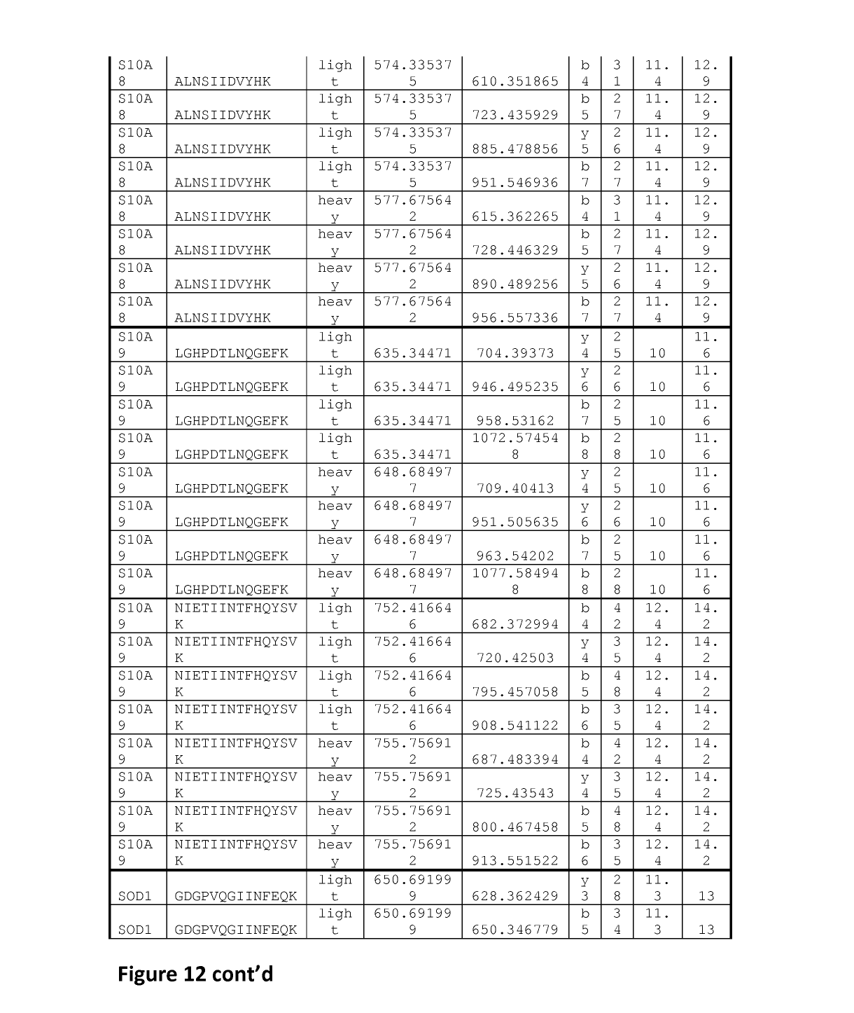

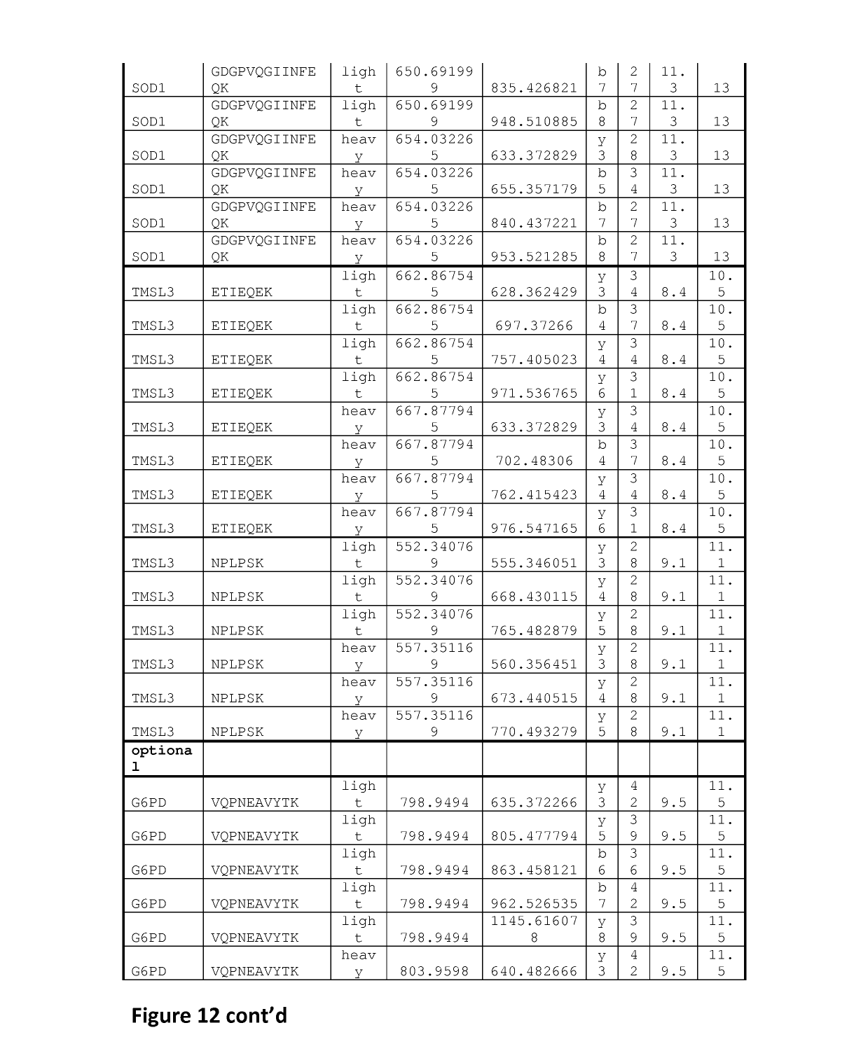

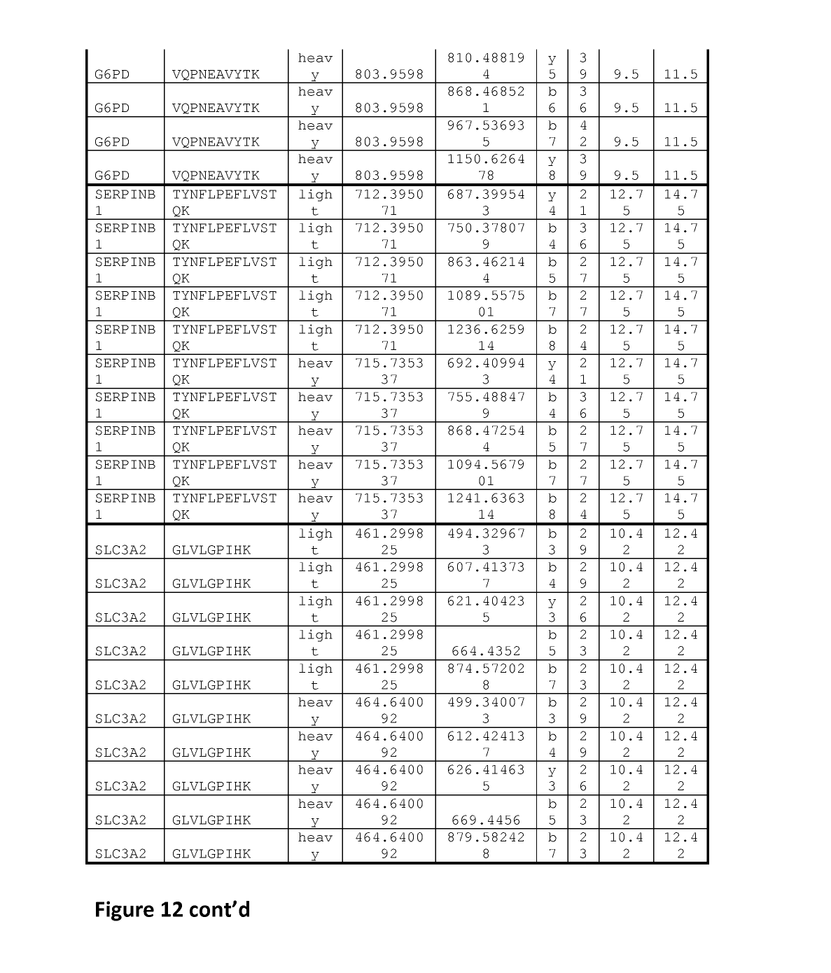

Using existing MS/MS data, most frequently observed specific peptides were selected for quantitation. If possible at least three peptides per protein were selected for SRM development. The representative peptides for eleven biomarker candidates are shown in Table 6 (FIG. 12). Criteria for selection included; no missed cleavages with trypsin and no variable modifications (in-vivo or experimental).

### Samples

MUTZ3 cells were exposed to sensitizer (4 μM DNCB, 150 μM TMA) and irritant (200 μM SDS) or were left untreated as described in the discovery study.

### Preparation of Samples

A pool sample was digested with trypsin and labelled with TMTsixplex to produce the heavy-labelled version of peptides to act as a reference for quantitation. Test samples were digested and labelled with TMTzero to produce the light labelled version of peptides. 15 μg each of the pool and test sample were afterwards mixed and underwent subsequent purification by solid-phase extraction and strong cation exchange using volatile buffers.

### SRM Analysis of Samples

The mixed heavy and light labelled samples were resuspended in 5% Acetonitril (=ACN), 0.2% Formic acid (=FA) and infused into an Accela 1250 Liquid Chromatography (LC) system coupled to a TSQ Vantage triple stage quadrupole mass spectrometer (Thermo Fisher) and SRM data was acquired. Corresponding TMTsixplex-labeled and TMTzero-labelled fragment ion masses were calculated and MS instrument parameters optimised for individual Q1 and Q3 transition pairs. A pooled cell lysate sample was digested, labelled with TMTsixplex and combined with the TMTzero-labeled reference peptides. Using accurate retention times for each peptide, the SRM cycle time was 1.5 seconds with retention time windows used to maximise the scan time given to each SRM transition. Including washes and time to equalibrate the column, the total run time of the method was 23 minutes. Declustering voltage was set to 5 Volt, Peak width (FWHM) was set to 0.5 and Chrome filter Peak width was set to 6 seconds. The SRM assay contains 153 SRM transitions, covering 19 peptides and 11 proteins. SRM transitions are listed in Table 6 (FIG. 12).

### Data Analysis

SRMs were visualised through Skyline version 1.2.0.3425 (https://skyline.gs.washington.edu/labkey/project/home/softwar e/Skyline/begin.view) and all peak matching visually verified. Peak areas were exported into Microsoft Excel. Transitions were summed to give a total intensity for all transitions for each peptide. The amount of endogenous (light) peptide is calculated based on the peak area ratio relative to the internal heavy-labeled reference sample.

### Results

The SRM multimarker assay was applied to distinguish samples treated with a chemical sensitizer from control and irritant samples based on specific protein response signatures. To test the performance of the multimarker panel comprising peptides specific for eight of the markers listed in Table 6 (FIG. 12) samples were exposed to typical contact (DNCB) and respiratory sensitizers (TMA) and irritant (SDS). For each sample (eight replicates per treatment) three analytical replicates were performed. The SRM peptide data were analyzed by analysis of variance (ANOVA) (P<0.05) followed by Tukey's post-hoc test (allergen compared to control, allergen versus irritant, P<0.05). Table 7 (FIG. 13) shows the performance of 20 selected peptides from protein precursors PGD, MPO, HSPA8, TMSL, S100A8, S100A9 and S100A4. With the exception of three peptides all peptides passed the significance criterion (p<0.05) and showed good performance to discriminate between allergen and control as individual peptides.

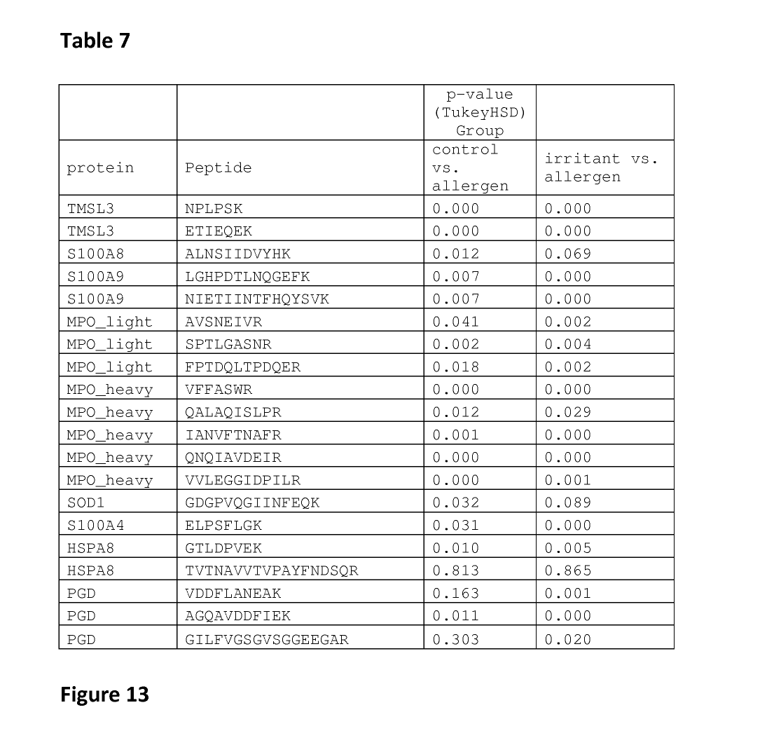

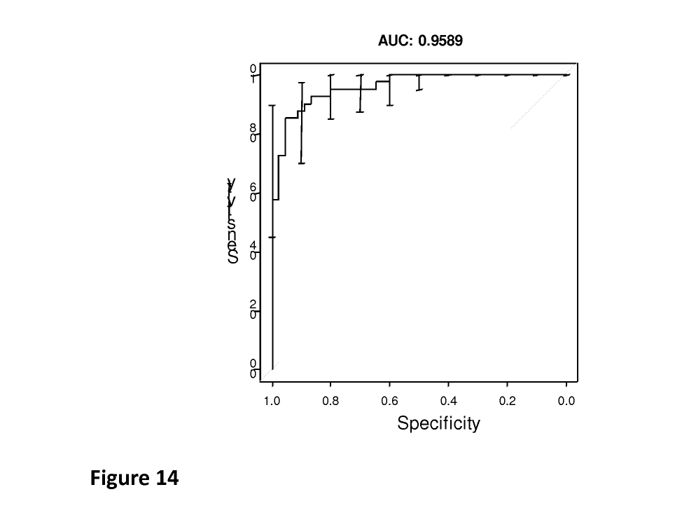

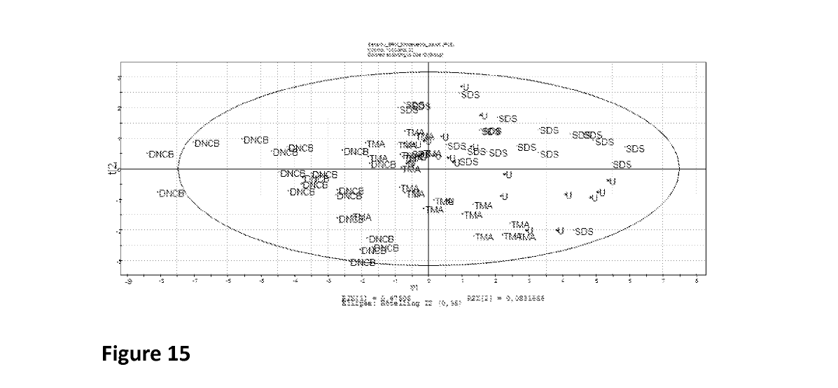

To maximize the potential of correctly identifying chemical sensitizers the effect of an eight marker panel on the area under the ROC curve (AUC) was calculated. As shown in FIG. 14 the eight marker panel showed excellent specificity and sensitivity to identify chemical sensitzers with an AUC of 0.96. To further demonstrate the power of this eight marker panel to differentiate chemicals in different classes the inventors performed a PLS-DA score plot of the samples, see FIG. 15. The four compounds used in the test set showed excellent separation based on the differential protein expression as determined by the TMT-SRM assay.

### REFERENCES

Gildea L A, Ryan C A, Foertsch L M, Kennedy J M, Dearman R J, Kimber I, Gerberick G F. (2006) Identification of gene expression changes induced by chemical allergens in dendritic cells: opportunities for skin sensitization testing. J Invest Dermatol. 126(8):1813-22.
Python F, Goebel C, Aeby P. (2009). Comparative DNA microarray analysis of human monocyte derived dendritic cells and MUTZ-3 cells exposed to the moderate skin sensitizer cinnamaldehyde. Toxicol Appl Pharmacol. 239(3):273-83.
Magnusson, B., Kligman, A. M., 1969. The identification of contact allergens by animal assay. The guinea pig maximization test. J. Invest. Dermatol. 52, 268-276.
Kimber, I., Hilton, J., Dearman, R. J., Gerberick, G. F., Ryan, C. A., Basketter, D. A., Scholes, E. W., Ladics, G. S., Loveless, S. E., House, R. V., Guy, A., 1995. An international evaluation of the murine local lymph node assay and comparison of modified procedures. Toxicology 103, 63-73.
Natsch A. (2010). The Nrf2-Keapl-ARE toxicity pathway as a cellular sensor for skin sensitizers—functional relevance and a hypothesis on innate reactions to skin sensitizers. Toxicol Sci. 113:284-92.
PBMC derived DCs
Tuschl H, Kovac R (2001). Langerhans cells and immature dendritic cells as model systems for screening of skin sensitizers. Toxicol In Vitro. (4-5):327-31.
Tuschl H, Kovac R, Weber E (2000). The expression of surface markers on dendritic cells as indicators for the sensitizing potential of chemicals. Toxicol In Vitro. (6):541-9.
De Smedt A C, Van Den Heuvel R L, Van Tendeloo V F, Berneman Z N, Schoeters G E, Weber E, Tuschl H. (2002). Phenotypic alterations and IL-lbeta production in CD34(+) progenitor- and monocyte-derived dendritic cells after exposure to allergens: a comparative analysis. Arch Dermatol Res. 294:109-116.
Ryan C A, Gildea L A, Hulette B C, Dearman R J, Kimber I, Gerberick G F (2004). Gene expression changes in peripheral blood-derived dendritic cells following exposure to a contact allergen. Toxicol Lett. 150(3):301-16.
Gildea L A, Ryan C A, Foertsch L M, Kennedy J M, Dearman R J, Kimber I, Gerberick G F (2006). Identification of gene expression changes induced by chemical allergens in dendritic cells: opportunities for skin sensitization testing. J Invest Dermatol. 126(8):1813-22
Schoeters E, Verheyen G R, Van Den Heuvel R, Nelissen I, Witters H, Van Tendeloo V F, Schoeters G E, Berneman Z N (2005). Expression analysis of immune-related genes in CD34(+) progenitor-derived dendritic cells after exposure to the chemical contact allergen DNCB. Toxicol In Vitro. 2005 19(7):909-13.
Schoeters E, Nuijten J M, Van Den Heuvel R L, Nelissen I, Witters H, Schoeters G E, Van Tendeloo V F, Berneman Z N, Verheyen G R (2006). Gene expression signatures in CD34+-progenitor-derived dendritic cells exposed to the chemical contact allergen nickel sulfate. Toxicol Appl Pharmacol.;216(1):131-49.
Schoeters E, Verheyen G R, Nelissen I, Van Rompay A R, Hooyberghs J, Van Den Heuvel R L, Witters H, Schoeters G E, Van Tendeloo V F, Berneman Z N (2007). Microarray analyses in dendritic cells reveal potential biomarkers for chemical-induced skin sensitization. Mol Immunol. 44(12):3222-33.
Hooyberghs J, Schoeters E, Lambrechts N, Nelissen I, Witters H, Schoeters G, Van Den Heuvel R. (2008). A cell-based in vitro alternative to identify skin sensitizers by gene expression. Toxicol Appl Pharmacol. 231(1):103-11
Lambrechts N, Vanheel H, Nelissen I, Witters H, Van Den Heuvel R, Van Tendeloo V, Schoeters G, Hooyberghs J. (2010). Assessment of chemical skin-sensitizing potency by an in vitro assay based on human dendritic cells. Toxicol Sci. 116(1):122-9.
Transcriptional profiling of DC-cell models (THP-1, Mutz-3): Verstraelen S, Nelissen I, Hooyberghs J, Witters H, Schoeters G, Van Cauwenberge P, Van Den Heuvel R. (2009) Gene profiles of THP-1 macrophages after in vitro exposure to respiratory (non-)sensitizing chemicals: identification of discriminating genetic markers and pathway analysis. Toxicol In Vitro.23(6):1151-62.
Lambrechts N, Verstraelen S, Lodewyckx H, Felicio A, Hooyberghs J, Witters H, Van Tendeloo V, Van Cauwenberge P, Nelissen I, Van Den Heuvel R, Schoeters G (2009). THP-1 monocytes but not macrophages as a potential alternative for CD34+ dendritic cells to identify chemical skin sensitizers. Toxicol Appl Pharmacol. 236(2):221-30.
Nelissen I, Selderslaghs I, Heuvel R V, Witters H, Verheyen G R, Schoeters G. (2009). MUTZ-3-derived dendritic cells as an in vitro alternative model to CD34+ progenitor-derived dendritic cells for testing of chemical sensitizers. Toxicol In Vitro. 23(8):1477-81.
Ott H, Wiederholt T, Bergstrom M A, Heise R, Skazik C, Czaja K, Marquardt Y, Karlberg A T, Merk H F, Baron J M. High-resolution transcriptional profiling of chemical-stimulated dendritic cells identifies immunogenic contact allergens, but not prohaptens. Skin Pharmacol Physiol. 2010; 23(4):213-24.
Williams E H, Williams C A, McLeod J D (2010). Identification of PDL-1 as a novel biomarker of sensitizer exposure in dendritic-like cells. Toxicol In Vitro. 24(6):1727-35.
Hu Z B, Ma W, Zaborski M, MacLeod R, Quentmeier H, Drexler H G (1996). Establishment and characterization of two novel cytokine-responsive acute myeloid and monocytic leukemia cell lines, MUTZ-2 and MUTZ-3. Leukemia.10(6):1025-40.
Visner G A, Dougall W C, Wilson J M, Burr I A, Nick H S (1990). Regulation of manganese superoxide dismutase by lipopolysaccharide, interleukin-1, and tumor necrosis factor. Role in the acute inflammatory response. J Biol Chem.265(5):2856-64
Rivollier A, Perrin-Cocon L, Luche S, Diemer H, Strub J M, Hanau D, van Dorsselaer A, Lotteau V, Rabourdin-Combe C, Rabilloud T, Servet-Delprat C (2006). High expression of antioxidant proteins in dendritic cells: possible implications in atherosclerosis. Mol Cell Proteomics. 5(4):726-36.
Byamba D, Kim T G, Kim D H, Je J H, Lee M G (2010). The Roles of Reactive Oxygen Species Produced by Contact Allergens and Irritants in Monocyte-derived Dendritic Cells. Ann Dermatol. 2010 22(3):269-78.
Ehrchen J M, Sunderkotter C, Foell D, Vogl T, Roth J (2009). The endogenous Toll-like receptor 4 agonist S100A8/S100A9 (calprotectin) as innate amplifier of infection, autoimmunity, and cancer. J Leukoc Biol. 86(3):557-66.
Martin S F, Dudda J C, Bachtanian E, Lembo A, Liller S, Durr C, Heimesaat M M, Bereswill S, Fejer G, Vassileva R, Jakob T, Freudenberg N, Termeer C C, Johner C, Galanos C, Freudenberg M A (2008). Toll-like receptor and IL-12 signaling control susceptibility to contact hypersensitivity. J Exp Med. 2008; 205(9):2151-62.

What is claimed is:

1. An in vitro method for determining the sensitizing potential of a test compound, comprising the steps of
(a) contacting said test compound with a cell;
(b) determining the presence or a change in the level of expression of a plurality of marker proteins selected from Table 1, Table 1 (A) Group 1, Table 1 (B) Group 2, Table 1 (C) Group 3 or a combination thereof, in said cell; and
(c) determining the sensitizing potential of said test compound based on said presence or change in level of expression wherein a change in the presence or level of expression of said one or more marker proteins is indicative of said test compound having sensitizing potential.

2. A method according to claim 1 wherein said cell is representative of a cell selected from the group consisting of skin, lung or immune system.

3. A method according to claim 2 wherein the said immune system cell is a dendritic cell.

4. A method according to claim 1 wherein said cell is from a human or animal cell line with dendritic-like properties.

5. A method according to claim 4 wherein said cell is from a lymphoid or myeloid cell line.

6. A method according to claim 5 wherein said cell line is selected from the group consisting of THP-1, U937 and Mutz-3 cells.

7. A method according to claim 1 wherein said plurality of marker proteins are selected from Table 1 (A) Group 1.

8. A method according to claim 1 wherein said plurality of marker proteins are selected from Table 1 (B) Group 2.

9. A method according to claim 1 wherein said plurality of marker proteins are selected from Table 5.

10. A method according to claim 1 wherein step (b) includes comparing the presence or level of expression of the plurality of protein markers with a reference level.

11. A method according to claim 1 wherein step (b) includes contacting the cell with at least one specific binding member that selectively binds to one of said plurality of marker protein or nucleic acid sequence encoding said marker protein; and
detecting and/or quantifying a complex formed by said specific binding member and the marker protein or nucleic acid sequence encoding said marker protein.

12. A method according to claim 1 wherein said determination step includes preparing a standard curve using standards of known expression levels of the plurality of marker proteins and comparing the reading obtained with the cell contacted with the test compound so as to derive a measure of the change in level of expression of the plurality of marker proteins.

13. A method according to claim 11 wherein said specific binding member may be an antibody or fragment thereof that specifically and selectively binds to said marker protein.

14. A method according to claim 13 wherein said specific binding member is an auto-antibody or fragment thereof that specifically and selectively binds to said marker protein wherein said auto-antibody is prepared from a blood sample obtained from a patient with skin or lung irritation or allergy.

15. A method according to claim 11 wherein the specific binding member is an aptamer.

16. A method according to claim 1 wherein step (b) is performed by mass spectrometry.

17. A method according to claim 1 wherein step (b) is performed by Selected Reaction Monitoring using one or more transitions for protein marker derived peptides; (i) comparing the peptide levels in the cell under test with peptide levels previously determined to represent sensitivity of the cell, and
(ii) determining the sensitizing potential of the test compound based on changes in expression of said plurality of marker proteins.

18. A method according to claim 17 wherein step (i) includes determining the amount of marker protein derived peptides from the cell under test with known amounts of corresponding synthetic peptides, wherein the synthetic peptides are identical in sequence to the peptides obtained from the cell except for a label.

19. A method according to claim 18 wherein the label is a tag of a different mass or a heavy isotope.

20. A method according to claim 1 wherein step (b) comprises determining the presence or amount of mRNA encoding said plurality of marker proteins in the cell following contact with the test compound.

21. A method according to claim 20 wherein the presence or amount of mRNA is determined using a primer or probe which selectively binds to the sequence of the protein marker encoding gene or complement thereof.

22. A method according to claim 11 wherein the binding member is immobilised on a solid support.

23-33. (canceled)

34. A method for the diagnosis or prognostic monitoring of contact or respiration sensitizing by an allergen or irritant on an individual exposed to said allergen or irritant the method comprising
(a) determining the presence or level of expression of a plurality of protein markers selected from Table 1, Table 1 (A) Group 1; Table 1 (B) Group 2; or Table 1 (c) Group 3 or a nucleic acid encoding any one or said protein markers or part thereof, in biological sample obtained from said individual.

35. A method according to claim 34 wherein the biological sample comprises cells selected from skin cells, lung cells, or immune system cells.

36. A method according to claim 34, wherein said plurality of marker proteins are selected from Table 5.

37-42. (canceled)
